SEQUENCE LISTING (14 sequences)

SEQ ID NO: 1 - protein, 213 aa, Artificial Sequence
  Description of Artificial Sequence Synthetic polypeptide
DIQMTQSPSSLSASVGDRVTITCKTSQDINKYMAWYQQTPGKAPRLLIHYTSALQPGIPSRFSGSGSGRDYTFTISSLQPEDIATYYCLQYDNLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 106 aa, Artificial Sequence [sequence omitted]

SEQ ID NO: 3 - protein, 11 aa, Artificial Sequence
  Description of Artificial Sequence Synthetic peptide
KTSQDINKYMA

SEQ ID NO: 4 - protein, 7 aa, Artificial Sequence
  Description of Artificial Sequence Synthetic peptide
YTSALQP

SEQ ID NO: 5 - protein, 8 aa, Artificial Sequence
  Description of Artificial Sequence Synthetic peptide
LQYDNLWT

SEQ ID NO: 6 - protein, 450 aa, Artificial Sequence
  Description of Artificial Sequence Synthetic polypeptide
  MOD_RES (1)..(1): Glutamine or pyroglutamic acid
  MOD_RES (450)..(450): May or may not be present
XVQLVQSGAEVKKPGASVKVSCKASGFNIKDTYIHWVRQAPGQRLEWMGRIDPANGYTKYDPKFQGRVTITADTSASTAYMELSSLRSEDTAVYYCAREGYYGNYGVYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 123 aa, Artificial Sequence
  Description of Artificial Sequence Synthetic polypeptide
  MOD_RES (1)..(1): Glutamine or pyroglutamic acid
XVQLVQSGAEVKKPGASVKVSCKASGFNIKDTYIHWVRQAPGQRLEWMGRIDPANGYTKYDPKFQGRVTITADTSASTAYMELSSLRSEDTAVYYCAREGYYGNYGVYAMDYWGQGTLVTVSS

SEQ ID NO: 8 - protein, 5 aa, Artificial Sequence
  Description of Artificial Sequence Synthetic peptide
DTYIH

SEQ ID NO: 9 - protein, 17 aa, Artificial Sequence
  Description of Artificial Sequence Synthetic peptide
RIDPANGYTKYDPKFQG

SEQ ID NO: 10 - protein, 14 aa, Artificial Sequence
  Description of Artificial Sequence Synthetic peptide
EGYYGNYGVYAMDY

SEQ ID NO: 11 - protein, 5 aa, Artificial Sequence
  Description of Artificial Sequence Synthetic peptide
EILDV

SEQ ID NO: 12 - protein, 8 aa, Artificial Sequence
  Description of Artificial Sequence Synthetic peptide
EILDVPST

SEQ ID NO: 13 - protein, 5 aa, Artificial Sequence
  Description of Artificial Sequence Synthetic peptide
LDVPS

SEQ ID NO: 14 - protein, 5 aa, Artificial Sequence
  Description of Artificial Sequence Synthetic peptide
  MOD_RES (4)..(4): 4-thioproline
RCDPC

(19) United States
(12) Patent Application Publication
(10) Pub. No.: US 2020/0255530 A1
(43) Pub. Date: Aug. 13, 2020

(54) COMPOSITIONS AND METHODS FOR TREATMENT OF STROKE AND OTHER CNS DISORDERS

(71) Applicant: BIOGEN MA INC., Cambridge, MA (US)

(72) Inventors: Lahar R. MEHTA, Redmond, WA (US); Bharath Kumar KANDADI MURALIDHARAN, Burlington, MA (US); Jacob ELKINS, Brookline, MA (US); Ivan NESTOROV, Acton, MA (US)

(73) Assignee: Biogen MA Inc., Cambridge, MA (US)

(21) Appl. No.: 15/746,733

(22) PCT Filed: Jul. 22, 2016
(86) PCT No.: PCT/US2016/043510

§ 371 Date: Jan. 22, 2018

Related U.S. Application Data
(60) Provisional application No. 62/196,270, filed on Jul. 23, 2015.

(51) Int. Cl. C07K 16/28; A61P 9/10

(52) U.S. Cl. CPC C07K 16/2842; A61K 2039/505; A61P 9/10

(57) ABSTRACT

The invention relates to methods of treating stroke comprising administration of a VLA-4 antagonist to a subject after the onset of the stroke, e.g., ischemic stroke (e.g., acute ischemic stroke) or hemorrhagic stroke (e.g., intracerebral hemorrhage), sub-arachnoid hemorrhage, or traumatic brain injury. Kits and articles of manufacture are also described herein.

### FIELD OF THE INVENTION

The invention relates to compositions and methods for treating stroke and/or other neurological deficits associated with stroke or traumatic brain injury.

### BACKGROUND OF INVENTION

Stroke is the second leading cause of death worldwide (fourth in the U.S., first in Japan). Nearly one-third of patients are left permanently disabled, with speech, movement, coordination, and cognition often affected. The annual stroke recurrence rate among survivors is 5%. The overall incidence of stroke is rising with the aging population (about 2% compound annual growth rate). Stroke places a heavy economic burden on patients, families, and the healthcare system.

Acute ischemic stroke (AIS) occurs when the brain does not receive adequate blood flow, typically due to occlusion of a blood vessel. Brain ischemia rapidly results in neuronal dysfunction and cell death. Over 1.7 million first-time AIS incidents occur each year in the seven major markets. AIS accounts for about 85% of all strokes (another type of stroke is hemorrhagic, which occurs when a weakened blood vessel ruptures causing bleeding into the surrounding brain tissue).

Current AIS therapies include intravenous (IV) thrombolysis and endovascular intervention. IV tissue plasminogen activator (tPA) (for thrombolysis) is the only approved agent for AIS and is effective in a limited time window (up to 3 hours in the U.S., 4.5 hours in Europe) after the last known normal (LKN). Functional measures, such as modified Rankin Scale (mRS) indicate a 15% relative improvement by tPA versus placebo. However, a risk of intracerebral hemorrhage and contra-indications (e.g., anti-coagulant use or uncontrolled hypertension) limits tPA use. About 5% of AIS patients are given tPA. Endovascular intervention includes surgical clot removal of a large vessel occlusion and has demonstrated modest efficacy. Eligiblity criteria and restriction to hospitals with expertise and surgical capabilities limits use broadly. Less than 2% of AIS patients undergo endovascular intervention. Thus, current AIS therapies are limited and suboptimal.

There is a need for more effective, safe/tolerable stroke (e.g., ischemic stroke, e.g, AIS, or hemorrhagic stroke) therapies that are available and useful to a larger percentage of stroke patients. There is also a need for therapies for subarachnoid hemorrhage and traumatic brain injury.

### SUMMARY OF INVENTION

The present invention provides, at least in part, methods and compositions for treating stroke, e.g., ischemic stroke (e.g., acute ischemic stroke (AIS)), hemorrhagic stroke (e.g., intracerebral hemorrhage, subarachnoid hemorrhage (SAH)), or traumatic brain injury (TBI).

In one aspect, provided herein is a method of treating a human subject having a stroke, e.g., an ischemic stroke, e.g., an acute ischemic stroke, a hemorrhagic stroke, e.g., intracerebral hemorrhage, a subarachnoid hemorrhage, or a traumatic brain injury (TBI), comprising:

administering a VLA-4 antagonist, e.g., an anti-alpha4 antibody molecule, e.g., a natalizumab-like antibody molecule, e.g., natalizumab, to the subject, at a dosage (e.g., as a single administration) of:
i) 250 to 350, or 300+/−5%, e.g., about 300, e.g., 300, mg; 350 to 500, e.g., 390 to 450, or 450+/−5%, or about 450, e.g., 450, mg; 550 to 650, e.g., 575 to 625, e.g., 600+/−5%, e.g., about 600, e.g., 600, mg;
ii) a dosage that results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L; or
iii) a dosage that results in an AUC that is comparable or higher than (e.g., within 5-10% of) the median exposure observed in lower body weight (e.g., body weight of <80 kg) or less severe stroke patients;
thereby treating the human subject.

In some embodiments, the dosage is 350 to 500 mg. In embodiments, the dosage is 390 to 450 mg. In embodiments, the dosage is 450+/−5% mg. In embodiments, the dosage is about 450 mg. In embodiments, the dosage is 450 mg. In embodiments, the dosage is 550 to 650 mg. In embodiments, the dosage is 575 to 625 mg. In embodiments, the dosage is 600+/−5% mg. In embodiments, the dosage is about 600 mg. In embodiments, the dosage is 600 mg.

In embodiments, the subject has a severe stroke, e.g., a stroke having a NIHSS score equal to or greater than 15 or 21. In embodiments, the subject has a stroke having a NIHSS score equal to or greater than 15. In embodiments, the subject has a stroke having a NIHSS score equal to or greater than 21.

In embodiments, the subject has a less than severe stroke, e.g., a stroke having a NIHSS score of less than 15. In embodiments, the subject has a mild stroke. In embodiments, the subject has a mild to moderate stroke.

In embodiments, the subject, at baseline, has an infarct size equal to or greater than 4.6 cm in diameter.

In embodiments, the subject, at baseline, has an infarct size less than 4.6 cm in diameter. In embodiments, the method comprises determining, e.g., determining prior to administration of the dosage, the severity of the stroke, e.g., determining if the patient has a severe stroke. In embodiments, determining comprises determining a NIHSS score, e.g., determining if the NIHSS score is equal to or greater than 15 or 21. In embodiments, determining comprises determining if the NIHSS score is equal to or greater than 15. In embodiments, determining comprises determining if the NIHSS score is equal to or greater than 21. In embodiments, determining comprises determining a NIHSS score, e.g., determining if the NIHSS score is less than 15. In embodiments, responsive to the determination of severity, a dosage of anti-VLA4 antagonist is selected.

In embodiments, administration of the dosage is initiated within 6 hours of last known normal. In embodiments, administration of the dosage is initiated within 9 hours, e.g., 6 to 9 hours, of last known normal. In embodiments, administration of the dosage is initiated within 12 hours, e.g., 6 to 12 hours, or 9 to 12 hours, of last known normal.

In embodiments, the method further comprises administering a subsequent dosage of the VLA-4 antagonist. In embodiments, the subsequent dosage is 125 to 175, 150+/−5%, about 150, or 150, mg. In embodiments, the subsequent dosage is 150 mg. In embodiments, the subsequent dosage is 250 to 350, or 300+/−5%, e.g., about 300, e.g., 300, mg. In embodiments, the subsequent dosage is 300 mg. In embodiments, the subsequent dosage is 350 to 500, e.g., 390 to 450, or 450+/−5%, or about 450, e.g., 450, mg. In embodiments, the subsequent dosage is 450 mg. In embodiments, the subsequent dosage is 550 to 650, e.g., 575 to 625, e.g., 600+/−5%, e.g., about 600, e.g., 600, mg. In embodiments, the subsequent dosage is 600 mg.

In embodiments, the subsequent dosage is administered on day 3 after the dosage (e.g., if the dosage is administered on the first day of the month the subsequent dosage is administered on the third day of the month. In embodiments, the subsequent dosage is administered on day 5 after the dosage. In embodiments, the subsequent dosage is administered on day 7 after the dosage. In embodiments, the subsequent dosage is administered four to six weeks after the dosage.

In embodiments, the VLA-4 antagonist comprises and antibody molecule that comprises CDR1, CDR2 and CDR3 from the light chain and CDR1, CDR2 and CDR3 from the heavy chain of natalizumab.

In embodiments, the VLA-4 antagonist comprises and antibody molecule that comprises the light chain variable region and the heavy chain variable region of natalizumab.

In embodiments, the VLA-4 antagonist comprises natalizumab.

In embodiments, the method comprises administering a VLA-4 antagonist to the subject within 12 hours or less, e.g., 10, 9, 8, 7, 6 hours or less, after LKN in the subject. In embodiments, the VLA-4 antagonist is administered within 9 hours or less after LKN, e.g., within 9, 8, 7, or 6 hours or less after LKN, or between 6 and 9 hours after LKN, or within 6 hours after LKN. In embodiments, the VLA-4 antagonist is administered within 6 hours or less after LKN, e.g., between 3 and 6 hours, 4.5 to 6 hours, 5 to 6 hours, after LKN. In embodiments, the method comprises administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject. In embodiments, the VLA-4 antagonist is administered within more than 2 to 9 hours or less after LKN, e.g., between 6 and 9 hours after LKN. In embodiments, the VLA-4 antagonist is administered within more than 2 hours to 6 hours or less after LKN, e.g., between 3 and 6 hours, 4.5 to 6 hours, 5 to 6 hours, after LKN.

In embodiments, the VLA-4 antagonist is an anti-VLA-4 antibody molecule, e.g., an anti-VLA-4 antibody molecule described herein. In embodiments, the anti-VLA-4 antibody molecule is a monoclonal, a humanized, a human, a chimeric anti-VLA-4 antibody molecule. In embodiments, the VLA-4 antagonist is an α4-binding fragment of an anti-VLA-4 antibody. In embodiments, the α4 binding fragment is an Fab, Fab′, F(ab′)₂, or Fv fragment. In embodiments, the anti-VLA-4 antibody molecule comprises one or more, preferably all, of HC CDR1, HC CDR2, HC CDR3, LC CDR1, LC CDR2 and LC CDR3 of natalizumab. In embodiments, the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

In embodiments, the stroke is a embolism-, thrombus- or hypoperfusion-associated stroke. In embodiments, the subject having the stroke does not have an intracranial hemorrhage.

In embodiments, the subject has not received a previous treatment with a VLA-4 antagonist, e.g., natalizumab. In embodiments, the subject does not have or is not at risk for developing progressive multifocal leukoencephalopathy (PML).

In embodiments, the VLA-4 antagonist is administered in combination with an additional agent or procedure. In embodiments, the VLA-4 antagonist is administered simultaneously with an additional agent or procedure. In embodiments, the VLA-4 antagonist is administered sequentially with an additional agent or procedure. In embodiments, the VLA-4 antagonist is administered, e.g., 30 minutes, 1 hour, 2 hours, 3 hours, 4 hours, 5 hours, or more, after the additional agent or procedure. In embodiments, the VLA-4 antagonist is administered, e.g., 30 minutes, 1 hour, 2 hours, 3 hours, 4 hours, 5 hours, or more, before the additional agent or procedure. In embodiments, the additional agent ameliorates one or more side effected associated with the administration of the VLA-4 antagonist, e.g., an agent which reduces or inhibits one or more symptom of hypersensitivity. In embodiments, the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine). In embodiments, the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

In embodiments, the VLA-4 antagonist is administered at a dosage and/or dosing schedule described herein. In embodiments, the VLA-4 antagonist is administered intravenously.

In an aspect, provided herein a method treating a human subject having a severe stroke, e.g., a severe ischemic stroke, e.g., a severe acute ischemic stroke, or a severe hemorrhagic stroke, e.g., a severe acute intracerebral hemorrhage, a severe subarachnoid hemorrhage, or severe TBI, comprising:

administering a VLA-4 antagonist, e.g., an anti-alpha4 antibody molecule, e.g., a natalizumab-like antibody molecule, e.g., natalizumab, to the subject,

thereby treating the human subject.

In embodiments, the method comprises determining, e.g., determining prior to administration of the VLA-4 antagonist, if the patient has a severe stroke. In embodiments, determining comprises determining a NIHSS score, e.g., determining if the NIHSS score is equal to or greater than 15 or 21. In embodiments, determining comprises determining if the NIHSS score is equal to or greater than 15. In embodiments, determining comprises determining if the NIHSS score is equal to or greater than 21.

In embodiments, the subject, e.g., at baseline, e.g., after stroke onset but prior to treatment with a natalizumab-like antibody molecule, e.g., natalizumab, has a NIHSS score of equal to or greater than 15 or 21, e.g., 21 to 26. In embodiments, the NIHSS score is equal to or greater than 15. In embodiments, the NIHSS score is equal to or greater than 21.

In embodiments, administration is initiated within 6 hours of last known normal. In embodiments, administration is initiated within 9 hours, e.g., 6 to 9 hours, of last known normal. In embodiments, administration is initiated within 12 hours, e.g., 6 to 12 hours, or 9 to 12 hours, of last known normal.

In embodiments, the method further comprises providing a subsequent administration of the VLA-4 antagonist to the subject.

In embodiments, the VLA-4 antagonist comprises and antibody molecule that comprises CDR1, CDR2 and CDR3 from the light chain and CDR1, CDR2 and CDR3 from the heavy chain of natalizumab.

In embodiments, the VLA-4 antagonist comprises and antibody molecule that comprises the light chain variable region and the heavy chain variable region of natalizumab.

In embodiments, the VLA-4 antagonist comprises natalizumab.

In embodiments, the method comprises administering a VLA-4 antagonist to the subject within 12 hours or less, e.g., 10, 9, 8, 7, 6 hours or less, after LKN in the subject. In embodiments, the VLA-4 antagonist is administered within 9 hours or less after LKN, e.g., within 9, 8, 7, or 6 hours or less after LKN, or between 6 and 9 hours after LKN, or within 6 hours after LKN. In embodiments, the VLA-4 antagonist is administered within 6 hours or less after LKN, e.g., between 3 and 6 hours, 4.5 to 6 hours, 5 to 6 hours, after LKN. In embodiments, the method comprises administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject. In embodiments, the VLA-4 antagonist is administered within more than 2 to 9 hours or less after LKN, e.g., between 6 and 9 hours after LKN. In embodiments, the VLA-4 antagonist is administered within more than 2 hours to 6 hours or less after LKN, e.g., between 3 and 6 hours, 4.5 to 6 hours, 5 to 6 hours, after LKN.

In embodiments, the VLA-4 antagonist is an anti-VLA-4 antibody molecule, e.g., an anti-VLA-4 antibody molecule described herein. In embodiments, the anti-VLA-4 antibody molecule is a monoclonal, a humanized, a human, a chimeric anti-VLA-4 antibody molecule. In embodiments, the VLA-4 antagonist is an α4-binding fragment of an anti-VLA-4 antibody. In embodiments, the α4 binding fragment is an Fab, Fab′, F(ab′)₂, or Fv fragment. In embodiments, the anti-VLA-4 antibody molecule comprises one or more, preferably all, of HC CDR1, HC CDR2, HC CDR3, LC CDR1, LC CDR2 and LC CDR3 of natalizumab.

In embodiments, the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

In embodiments, the stroke is a embolism-, thrombus- or hypoperfusion-associated stroke. In embodiments, the subject having the stroke does not have an intracranial hemorrhage.

In embodiments, the subject has not received a previous treatment with a VLA-4 antagonist, e.g., natalizumab. In embodiments, the subject does not have or is not at risk for developing progressive multifocal leukoencephalopathy (PML).

In embodiments, the VLA-4 antagonist is administered in combination with an additional agent or procedure. In embodiments, the VLA-4 antagonist is administered simultaneously with an additional agent or procedure. In embodiments, the VLA-4 antagonist is administered sequentially with an additional agent or procedure. In embodiments, the VLA-4 antagonist is administered, e.g., 30 minutes, 1 hour, 2 hours, 3 hours, 4 hours, 5 hours, or more, after the additional agent or procedure. In embodiments, the VLA-4 antagonist is administered, e.g., 30 minutes, 1 hour, 2 hours, 3 hours, 4 hours, 5 hours, or more, before the additional agent or procedure. In embodiments, the additional agent ameliorates one or more side effected associated with the administration of the VLA-4 antagonist, e.g., an agent which reduces or inhibits one or more symptom of hypersensitivity. In embodiments, the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine). In embodiments, the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

In embodiments, the VLA-4 antagonist is administered at a dosage and/or dosing schedule described herein. In embodiments, the VLA-4 antagonist is administered intravenously.

In an aspect, provided herein is a method of treating a human subject having a stroke, e.g., an ischemic stroke, e.g., an acute ischemic stroke, or a hemorrhagic stroke, e.g., an intracerebral hemorrhage, a subarachnoid hemorrhage, or a traumatic brain injury (TBI), comprising:

administering a VLA-4 antagonist, e.g., an anti-alpha4 antibody molecule, e.g., a natalizumab like antibody molecule, e.g., natalizumab, to the subject, at a dosage of
i) 250 to 390, e.g., 275 to 325, e.g., 300+/−5%, or about 300, e.g., 300, mg;
ii) a dosage that results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L; or
iii) a dosage that results in an AUC that is comparable or higher than (e.g., within 5-10% of) the median exposure observed in lower body weight (e.g., body weight of <80 kg) or less severe stroke patients,
thereby treating the human subject.

In embodiments, the method comprises determining, e.g., determining prior to administration of the VLA-4 antagonist, if the patient has a less than severe stroke. In embodiments, determining comprises determining NIHSS score, e.g., determining if the NIHSS score is less than 15. In embodiments, the subject has a less than severe stroke. In embodiments, the subject has a mild stroke. In embodiments, the subject has a mild to moderate stroke.

In embodiments, the subject, e.g., at baseline, e.g., after stroke onset but prior to treatment with a natalizumab-like antibody molecule, e.g., natalizumab, has a NIHSS score of 3 to 7. In embodiments, the subject, e.g., at baseline, e.g., after stroke onset but prior to treatment with a natalizumab-like antibody molecule, e.g., natalizumab, has a NIHSS score of 8 to 10. In embodiments, the subject, e.g., at baseline, e.g., after stroke onset but prior to treatment with a natalizumab-like antibody molecule, e.g., natalizumab, has a NIHSS score of 11 to 14. In embodiments, the subject, e.g., at baseline, e.g., after stroke onset but prior to treatment with a natalizumab-like antibody molecule, e.g., natalizumab, has a NIHSS score of 15 to 14.

In embodiments, administration is initiated within 6 hours of last known normal. In embodiments, administration is initiated within 9 hours, e.g., 6 to 9 hours, of last known normal. In embodiments, administration is initiated within 12 hours, e.g., 6 to 12 hours, or 9 to 12 hours, of last known normal.

In embodiments, the dosage is about 300 mg.

In embodiments, the VLA-4 antagonist comprises and antibody molecule that comprises CDR1, CDR2 and CDR3 from the light chain and CDR1, CDR2 and CDR3 from the heavy chain of natalizumab. In embodiments, the VLA-4 antagonist comprises and antibody molecule that comprises the light chain variable region and the heavy chain variable region of natalizumab. In embodiments, the VLA-4 antagonist comprises natalizumab.

In embodiments, the method comprises administering a VLA-4 antagonist to the subject within 12 hours or less, e.g., 10, 9, 8, 7, 6 hours or less, after LKN in the subject. In embodiments, the VLA-4 antagonist is administered within 9 hours or less after LKN, e.g., within 9, 8, 7, or 6 hours or less after LKN, or between 6 and 9 hours after LKN, or within 6 hours after LKN. In embodiments, the VLA-4 antagonist is administered within 6 hours or less after LKN, e.g., between 3 and 6 hours, 4.5 to 6 hours, 5 to 6 hours, after LKN. In embodiments, the method comprises administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject. In embodiments, the VLA-4 antagonist is administered within more than 2 to 9 hours or less after LKN, e.g., between 6 and 9 hours after LKN. In embodiments, the VLA-4 antagonist is administered within more than 2 hours to 6 hours or less after LKN, e.g., between 3 and 6 hours, 4.5 to 6 hours, 5 to 6 hours, after LKN.

In embodiments, the VLA-4 antagonist is an anti-VLA-4 antibody molecule, e.g., an anti-VLA-4 antibody molecule described herein. In embodiments, the anti-VLA-4 antibody molecule is a monoclonal, a humanized, a human, a chimeric anti-VLA-4 antibody molecule. In embodiments, the VLA-4 antagonist is an α4-binding fragment of an anti-VLA-4 antibody. In embodiments, the α4 binding fragment is an Fab, Fab′, F(ab′)₂, or Fv fragment. In embodiments, the anti-VLA-4 antibody molecule comprises one or more, preferably all, of HC CDR1, HC CDR2, HC CDR3, LC CDR1, LC CDR2 and LC CDR3 of natalizumab.

In embodiments, the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

In embodiments, the stroke is a embolism-, thrombus- or hypoperfusion-associated stroke. In embodiments, the subject having the stroke does not have an intracranial hemorrhage.

In embodiments, the subject has not received a previous treatment with a VLA-4 antagonist, e.g., natalizumab.

In embodiments, the subject does not have or is not at risk for developing progressive multifocal leukoencephalopathy (PML).

In embodiments, the VLA-4 antagonist is administered in combination with an additional agent or procedure. In embodiments, the VLA-4 antagonist is administered simultaneously with an additional agent or procedure. In embodiments, the VLA-4 antagonist is administered sequentially with an additional agent or procedure. In embodiments, the VLA-4 antagonist is administered, e.g., 30 minutes, 1 hour, 2 hours, 3 hours, 4 hours, 5 hours, or more, after the additional agent or procedure. In embodiments, the VLA-4 antagonist is administered, e.g., 30 minutes, 1 hour, 2 hours, 3 hours, 4 hours, 5 hours, or more, before the additional agent or procedure. In embodiments, the additional agent ameliorates one or more side effected associated with the administration of the VLA-4 antagonist, e.g., an agent which reduces or inhibits one or more symptom of hypersensitivity. In embodiments, the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine). In embodiments, the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

In embodiments, the VLA-4 antagonist is administered at a dosage and/or dosing schedule described herein. In embodiments, the VLA-4 antagonist is administered intravenously.

In as aspect, provided herein is a method of treating a human subject having a less than severe stroke, e.g., a less than severe ischemic stroke, e.g., a less than severe acute ischemic stroke, or a less than severe hemorrhagic stroke, e.g., a less than severe intracerebral hemorrhage, a less than severe subarachnoid hemorrhage, or a less than severe TBI, comprising:

administering a VLA-4 antagonist, e.g., an anti-alpha4 antibody molecule, e.g., a natalizumab-like antibody molecule, e.g., natalizumab, to the subject.

thereby treating the human subject.

In embodiments, the subject has a mild stroke. In embodiments, the subject has a mild to moderate stroke.

In embodiments, the method comprises determining, e.g., determining prior to administration of the VLA-4 antagonist, if the patient has a less than severe stroke. In embodiments, determining comprises determining NIHSS score, e.g., determining if the NIHSS score is less than 15.

In embodiments, the subject, e.g., at baseline, e.g., after stroke onset but prior to treatment with the VLA-4 antagonist has a NIHSS score of 3 to 7. In embodiments, the subject, e.g., at baseline, e.g., after stroke onset but prior to treatment with the VLA-4 antagonist has a NIHSS score of 8 to 10. In embodiments, the subject, e.g., at baseline, e.g., after stroke onset but prior to treatment with the VLA-4 antagonist has a NIHSS score of 11 to 14.

In embodiments, administration is initiated within 6 hours of last known normal. In embodiments, administration is initiated within 9 hours, e.g., 6 to 9 hours, of last known normal. In embodiments, administration is initiated within 12 hours, e.g., 6 to 12 hours, or 9 to 12 hours, of last known normal.

In embodiments, the method further comprises providing a subsequent administration of the VLA-4 antagonist to the subject.

In embodiments, the VLA-4 antagonist comprises and antibody molecule that comprises CDR1, CDR2 and CDR3 from the light chain and CDR1, CDR2 and CDR3 from the heavy chain of natalizumab. In embodiments, the VLA-4 antagonist comprises and antibody molecule that comprises the light chain variable region and the heavy chain variable region of natalizumab. In embodiments, the VLA-4 antagonist comprises natalizumab.

In embodiments, the method comprises administering a VLA-4 antagonist to the subject within 12 hours or less, e.g., 10, 9, 8, 7, 6 hours or less, after LKN in the subject. In embodiments, the VLA-4 antagonist is administered within 9 hours or less after LKN, e.g., within 9, 8, 7, or 6 hours or less after LKN, or between 6 and 9 hours after LKN, or within 6 hours after LKN. In embodiments, the VLA-4 antagonist is administered within 6 hours or less after LKN, e.g., between 3 and 6 hours, 4.5 to 6 hours, 5 to 6 hours, after LKN. In embodiments, the method comprises administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject. In embodiments, the VLA-4 antagonist is administered within more than 2 to 9 hours or less after LKN, e.g., between 6 and 9 hours after LKN. In embodiments, the VLA-4 antagonist is administered within more than 2 hours to 6 hours or less after LKN, e.g., between 3 and 6 hours, 4.5 to 6 hours, 5 to 6 hours, after LKN.

In embodiments, the VLA-4 antagonist is an anti-VLA-4 antibody molecule, e.g., an anti-VLA-4 antibody molecule described herein. In embodiments, the anti-VLA-4 antibody molecule is a monoclonal, a humanized, a human, a chimeric anti-VLA-4 antibody molecule. In embodiments, the VLA-4 antagonist is an α4-binding fragment of an anti-VLA-4 antibody. In embodiments, the α4 binding fragment is an Fab, Fab′, F(ab′)₂, or Fv fragment. In embodiments, the anti-VLA-4 antibody molecule comprises one or more, preferably all, of HC CDR1, HC CDR2, HC CDR3, LC CDR1, LC CDR2 and LC CDR3 of natalizumab.

In embodiments, the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

In embodiments, the stroke is a embolism-, thrombus- or hypoperfusion-associated stroke. In embodiments, the subject having the stroke does not have an intracranial hemorrhage.

In embodiments, the subject has not received a previous treatment with a VLA-4 antagonist, e.g., natalizumab.

In embodiments, the subject does not have or is not at risk for developing progressive multifocal leukoencephalopathy (PML).

In embodiments, the VLA-4 antagonist is administered in combination with an additional agent or procedure. In embodiments, the VLA-4 antagonist is administered simultaneously with an additional agent or procedure. In embodiments, the VLA-4 antagonist is administered sequentially with an additional agent or procedure. In embodiments, the VLA-4 antagonist is administered, e.g., 30 minutes, 1 hour, 2 hours, 3 hours, 4 hours, 5 hours, or more, after the additional agent or procedure. In embodiments, the VLA-4 antagonist is administered, e.g., 30 minutes, 1 hour, 2 hours, 3 hours, 4 hours, 5 hours, or more, before the additional agent or procedure. In embodiments, the additional agent ameliorates one or more side effected associated with the administration of the VLA-4 antagonist, e.g., an agent which reduces or inhibits one or more symptom of hypersensitivity. In embodiments, the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine). In embodiments, the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

In embodiments, the VLA-4 antagonist is administered at a dosage and/or dosing schedule described herein. In embodiments, the VLA-4 antagonist is administered intravenously.

In an aspect, provided herein is a method of treating a human subject having a stroke, e.g., an ischemic stroke, e.g., an acute ischemic stroke, or hemorrhagic stroke, e.g., an intracerebral hemorrhage, a subarachnoid hemorrhage, or a traumatic brain injury (TBI), comprising:

determining the severity of a stroke, e.g., by receiving information, e.g., from a third party, on the severity of the stroke (or TBI), and responsive to that determination, selecting a dosage of a VLA-4 antagonist, e.g., an anti-alpha4 antibody molecule, e.g., a natalizumab-like antibody molecule, e.g., natalizumab,

thereby treating the human subject.

In embodiments, the method further comprises administering the selected dosage to the subject.

In embodiments, the method comprises, if the stroke is determined to be a severe stroke, selecting and/or administering a dosage-A of the VLA-4 antagonist and if the stroke is determined to be a less than severe stroke, selecting and or administering a dosage-B, e.g., wherein the dosage-A is higher than dosage-B, e.g., is at least 10, 20 or 30% higher.

In embodiments, dosage-A is 350 to 500, e.g., 390 to 450, or 450+/−5%, or about 450, e.g., 450, mg; 550 to 650, e.g., 575 to 625, e.g., 600+/−5%, e.g., about 600, e.g., 600, mg. In embodiments, dosage-A is 350 to 500 mg. In embodiments, dosage-A is 390 to 450 mg. In embodiments, dosage-A is 450+/−5% mg. In embodiments, dosage-A is about 450 mg. In embodiments, dosage-A is 450 mg. In embodiments, dosage-A is 550 to 650 mg. In embodiments, dosage-A is 575 to 625 mg. In embodiments, dosage-A is 600+/−5% mg. In embodiments, dosage-A is about 600 mg. In embodiments, dosage-A is 600 mg. In embodiments, dosage-A results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L; or wherein dosage-A is results in an AUC that is comparable or higher than (e.g., within 5-10% of) the median exposure observed in lower body weight (e.g., body weight of <80 kg) or less severe stroke patients.

In embodiments, dosage-B is 250 to 390, e.g., 275 to 325, e.g., 300+/−5%, or about 300, e.g., 300, mgs. In embodiments, dosage-B is 250 to 390 mg. In embodiments, dosage-B is 275 to 325 mg. In embodiments, dosage-B is 300+/−5% mg. In embodiments, dosage-B is about 300 mg. In embodiments, dosage-B is 300 mg. In embodiments, dosage-B results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000, mg*hr/L; or wherein dosage-B is results in an AUC that is comparable or higher than (e.g., within 5-10% of) the median exposure observed in lower body weight (e.g., body weight of <80 kg) or less severe stroke patients.

In embodiments, determining comprises determining a NIHSS score, e.g., determining if the NIHSS score is equal to or greater than 15 or 21. In embodiments, determining comprises determining if the NIHSS score is equal to or greater than 15. In embodiments, determining comprises determining if the NIHSS score is equal to or greater than 21.

In embodiments, the method comprises determining that the stroke is a severe stroke. In embodiments, the subject, e.g., at baseline, e.g., after stroke onset but prior to treatment with a natalizumab-like antibody molecule, e.g., natalizumab, has a NIHSS score of 21 or greater, e.g., 21 to 26. In embodiments, the NIHSS score is equal to or greater than 15. In embodiments, the NIHSS score is equal to or greater than 21.

In embodiments, determining comprises determining NIHSS score, e.g., determining if the NIHSS score is less than 15.

In embodiments, the stroke is determined to be less than severe. In embodiments, the stroke is mild stroke. In embodiments, the stroke is a mild to moderate stroke.

In embodiments, the subject, e.g., at baseline, e.g., after stroke onset but prior to treatment with a natalizumab-like antibody molecule, e.g., natalizumab, has a NIHSS score of 3 to 7. In embodiments, the subject, e.g., at baseline, e.g., after stroke onset but prior to treatment with a natalizumab-like antibody molecule, e.g., natalizumab, has a NIHSS score of 8 to 10. In embodiments, the subject, e.g., at baseline, e.g., after stroke onset but prior to treatment with a natalizumab-like antibody molecule, e.g., natalizumab, has a NIHSS score of 11 to 14. In embodiments, the subject, e.g., at baseline, e.g., after stroke onset but prior to treatment with a natalizumab-like antibody molecule, e.g., natalizumab, has a NIHSS score of 5 to 14.

In embodiments, administration is initiated within 6 hours of last known normal. In embodiments, administration is initiated within 9 hours, e.g., 6 to 9 hours, of last known normal. In embodiments, administration is initiated within 12 hours, e.g., 6 to 12 hours, or 9 to 12 hours, of last known normal.

In embodiments, the VLA-4 antagonist comprises and antibody molecule that comprises CDR1, CDR2 and CDR3 from the light chain and CDR1, CDR2 and CDR3 from the heavy chain of natalizumab. In embodiments, he VLA-4 antagonist comprises and antibody molecule that comprises the light chain variable region and the heavy chain variable region of natalizumab. In embodiments, the VLA-4 antagonist comprises natalizumab.

In embodiments, the method comprises administering a VLA-4 antagonist to the subject within 12 hours or less, e.g., 10, 9, 8, 7, 6 hours or less, after LKN in the subject. In embodiments, the VLA-4 antagonist is administered within 9 hours or less after LKN, e.g., within 9, 8, 7, or 6 hours or less after LKN, or between 6 and 9 hours after LKN, or within 6 hours after LKN. In embodiments, the VLA-4 antagonist is administered within 6 hours or less after LKN, e.g., between 3 and 6 hours, 4.5 to 6 hours, 5 to 6 hours, after LKN. In embodiments, the method comprises administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject. In embodiments, the VLA-4 antagonist is administered within more than 2 to 9 hours or less after LKN, e.g., between 6 and 9 hours after LKN. In embodiments, the VLA-4 antagonist is administered within more than 2 hours to 6 hours or less after LKN, e.g., between 3 and 6 hours, 4.5 to 6 hours, 5 to 6 hours, after LKN.

In embodiments, the VLA-4 antagonist is an anti-VLA-4 antibody molecule, e.g., an anti-VLA-4 antibody molecule described herein. In embodiments, the anti-VLA-4 antibody molecule is a monoclonal, a humanized, a human, a chimeric anti-VLA-4 antibody molecule. In embodiments, the VLA-4 antagonist is an α4-binding fragment of an anti-VLA-4 antibody. In embodiments, the α4 binding fragment is an Fab, Fab′, F(ab′)₂, or Fv fragment. In embodiments, the anti-VLA-4 antibody molecule comprises one or more, preferably all, of HC CDR1, HC CDR2, HC CDR3, LC CDR1, LC CDR2 and LC CDR3 of natalizumab.

In embodiments, the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

In embodiments, the stroke is a embolism-, thrombus- or hypoperfusion-associated stroke.

In embodiments, the subject having the stroke does not have an intracranial hemorrhage.

In embodiments, the subject has not received a previous treatment with a VLA-4 antagonist, e.g., natalizumab.

In embodiments, the subject does not have or is not at risk for developing progressive multifocal leukoencephalopathy (PML).

In embodiments, the VLA-4 antagonist is administered in combination with an additional agent or procedure. In embodiments, the VLA-4 antagonist is administered simultaneously with an additional agent or procedure. In embodiments, the VLA-4 antagonist is administered sequentially with an additional agent or procedure. In embodiments, the VLA-4 antagonist is administered, e.g., 30 minutes, 1 hour, 2 hours, 3 hours, 4 hours, 5 hours, or more, after the additional agent or procedure. In embodiments, the VLA-4 antagonist is administered, e.g., 30 minutes, 1 hour, 2 hours, 3 hours, 4 hours, 5 hours, or more, before the additional agent or procedure. In embodiments, the additional agent ameliorates one or more side effected associated with the administration of the VLA-4 antagonist, e.g., an agent which reduces or inhibits one or more symptom of hypersensitivity. In embodiments, the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine).

In embodiments, the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

In embodiments, the VLA-4 antagonist is administered at a dosage and/or dosing schedule described herein. In embodiments, the VLA-4 antagonist is administered intravenously.

In an aspect, provided herein is a method of treating a human subject having a stroke, e.g., an ischemic stroke, e.g., an acute ischemic stroke, or a hemorrhagic stroke, e.g., an intracerebral hemorrhage, a subarachnoid hemorrhage, or a traumatic brain injury (TBI), comprising:

administering a first dosage of a VLA-4 antagonist to the subject at a time t1, administering a second dosage of a VLA-4 antagonist to the subject at a time t2, and (optionally) administering a third dosage of a VLA-4 antagonist to the subject at a time t3, thereby treating the subject.

In embodiments, the method comprises administering a third dosage of a VLA-4 antagonist to the subject at a time t3.

In embodiments, the three dosages result in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L. In embodiments, the three dosages result in an AUC that is comparable to or higher than (e.g., within 5-10% of) the median AUC of a less than severe stroke patient or a patient having a lower body weight (e.g., of <80 kg).

In embodiments, the first dosage is greater than one or both of the second and third dosages. In embodiments, the first dosage is the same as one or both of the second and third dosages, e.g., wherein all three dosages are the same.

In embodiments, t1 is the day (i.e., within 24 hours) of diagnosis.

In embodiments, at least one or more days (e.g., 1, 2, 3, 4, 5, or more days) are interposed between t1 and t2. In embodiments, about 2 days (e.g., 36-60 hours, e.g., 40-56 hours, e.g., 44-52 hours, e.g., about 48 hours) are interposed between t1 and t2. In embodiments, at least one or more days (e.g., 1, 2, 3, 4, 5, or more days) are interposed between t2 and t3. In embodiments, about 2 days (e.g., 36-60 hours, e.g., 40-56 hours, e.g., 44-52 hours, e.g., about 48 hours) are interposed between t2 and t3.

In embodiments, t1 is the day on which the first dosage is administered; g2 is the third day after t1, (e.g., if t1 is a Monday, then t2 is the first following Wednesday); and t3 is the fifth day after t1.

In embodiments, the first dosage is 250 to 350 mg, the second dosage is 100 to 200 mg, and the third dosage is 100 to 200 mg. In embodiments, the first dosage is 275 to 325 mg, the second dosage is 125 mg to 175 mg, and the third dosage is 125 to 175 mg. In embodiments, the first dosage is 300+/−5% mg, the second dosage is 150+/−5% mg, and the third dosage is 150+/−5% mg. In embodiments, the first dosage is about 300 mg, the second dosage is about 150 mg, and the third dosage is about 150 mg. In embodiments, the first dosage is 300 mg, the second dosage is 150 mg, and the third dosage is 150 mg. In embodiments, the first, second and third dosage, each is 100 to 200 mg. In embodiments, the first, second and third dosage, each is 125 to 175 mg. In embodiments, the first, second and third dosage, each is 150+/−5% mg. In embodiments, the first, second and third dosage, each is about 150 mg. In embodiments, the first, second and third dosage, each is 150 mg.

In embodiments, the subject has a severe stroke, e.g., a stroke having a NIHSS score equal to or greater than 15 or 21. In embodiments, the NIHSS score is equal to or greater than 15. In embodiments, the NIHSS score is equal to or greater than 21.

In embodiments, the subject has a less than severe stroke, e.g., a stroke having a NIHSS score of less than 15. In embodiments, the subject has a mild stroke. In embodiments, the subject has a mild to moderate stroke.

In embodiments, the method comprises determining, e.g., determining prior to administration of the VLA-4 antagonist, the severity of the stroke, e.g., determining if the patient has a severe stroke.

In embodiments, determining comprises determining a NIHSS score, e.g., determining if the NIHSS score is equal to or greater than 15 or 21. In embodiments, determining comprises determining if the NIHSS score is equal to or greater than 15. In embodiments, determining comprises determining if the NIHSS score is equal to or greater than 21.

In embodiments, the method comprises determining, e.g., determining prior to administration of the VLA-4 antagonist, the severity of the stroke, e.g., determining if the patient has less than severe stroke.

In embodiments, determining comprises determining a NIHSS score, e.g., determining if the NIHSS score is less than 15.

In embodiments, responsive to the determination of severity, a dosage of anti-VLA4 antagonist is selected.

In embodiments, administration of the first dosage is initiated within 6 hours of last known normal. In embodiments, administration of the first dosage is initiated within 9 hours, e.g., 6 to 9 hours, of last known normal. In embodiments, administration of the first dosage is initiated within 12 hours, e.g., 6 to 12 hours, or 9 to 12 hours, of last known normal.

In embodiments, the VLA-4 antagonist comprises and antibody molecule that comprises CDR1, CDR2 and CDR3 from the light chain and CDR1, CDR2 and CDR3 from the heavy chain of natalizumab. In embodiments, the VLA-4 antagonist comprises and antibody molecule that comprises the light chain variable region and the heavy chain variable region of natalizumab. In embodiments, the VLA-4 antagonist comprises natalizumab.

In embodiments, the method comprises administering a VLA-4 antagonist to the subject within 12 hours or less, e.g., 10, 9, 8, 7, 6 hours or less, after LKN in the subject. In embodiments, the VLA-4 antagonist is administered within 9 hours or less after LKN, e.g., within 9, 8, 7, or 6 hours or less after LKN, or between 6 and 9 hours after LKN, or within 6 hours after LKN. In embodiments, the VLA-4 antagonist is administered within 6 hours or less after LKN, e.g., between 3 and 6 hours, 4.5 to 6 hours, 5 to 6 hours, after LKN. In embodiments, the method comprises administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject. In embodiments, the VLA-4 antagonist is administered within more than 2 to 9 hours or less after LKN, e.g., between 6 and 9 hours after LKN. In embodiments, the VLA-4 antagonist is administered within more than 2 hours to 6 hours or less after LKN, e.g., between 3 and 6 hours, 4.5 to 6 hours, 5 to 6 hours, after LKN.

In embodiments, the VLA-4 antagonist is an anti-VLA-4 antibody molecule, e.g., an anti-VLA-4 antibody molecule described herein. In embodiments, the anti-VLA-4 antibody molecule is a monoclonal, a humanized, a human, a chimeric anti-VLA-4 antibody molecule. In embodiments, the VLA-4 antagonist is an α4-binding fragment of an anti-VLA-4 antibody. In embodiments, the α4 binding fragment is an Fab, Fab′, F(ab′)₂, or Fv fragment. In embodiments, the anti-VLA-4 antibody molecule comprises one or more, preferably all, of HC CDR1, HC CDR2, HC CDR3, LC CDR1, LC CDR2 and LC CDR3 of natalizumab.

In embodiments, the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

In embodiments, the stroke is a embolism-, thrombus- or hypoperfusion-associated stroke.

In embodiments, the subject having the stroke does not have an intracranial hemorrhage.

In embodiments, the subject has not received a previous treatment with a VLA-4 antagonist, e.g., natalizumab.

In embodiments, the subject does not have or is not at risk for developing progressive multifocal leukoencephalopathy (PML).

In embodiments, the VLA-4 antagonist is administered in combination with an additional agent or procedure. In embodiments, the VLA-4 antagonist is administered simultaneously with an additional agent or procedure. In embodiments, the VLA-4 antagonist is administered sequentially with an additional agent or procedure. In embodiments, the VLA-4 antagonist is administered, e.g., 30 minutes, 1 hour, 2 hours, 3 hours, 4 hours, 5 hours, or more, after the additional agent or procedure. In embodiments, the VLA-4 antagonist is administered, e.g., 30 minutes, 1 hour, 2 hours, 3 hours, 4 hours, 5 hours, or more, before the additional agent or procedure. In embodiments, the additional agent ameliorates one or more side effected associated with the administration of the VLA-4 antagonist, e.g., an agent which reduces or inhibits one or more symptom of hypersensitivity. In embodiments, the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine).

In embodiments, the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

In embodiments, the VLA-4 antagonist is administered at a dosage and/or dosing schedule described herein. In embodiments, the VLA-4 antagonist is administered intravenously.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description, and from the claims.

### BRIEF DESCRIPTION OF THE FIGURES

FIG. 1 is a schematic showing the Phase II trial protocol described in the Examples section.

FIG. 2 is a graph showing the median infarct volume over time in patients treated with natalizumab or placebo.

FIG. 3 is a set of schematics showing the functional outcomes (modified Rankin Scale scores) of patients treated with natalizumab or placebo at 30 or 90 days.

FIG. 4 is a graph showing the median Barthel's index (BI) scores over time in patients treated with natalizumab or placebo.

FIG. 5 is a graph showing the median NIHSS scores over time of patients treated with natalizumab or placebo.

FIG. 6 is a graph showing the median SIS-16 scores over time of patients treated with natalizumab or placebo.

FIG. 7 is a graph showing the median Montreal Cognitive Assessment (MoCA) scores over time of patients treated with natalizumab or placebo.

FIG. 8 is a set of graphs showing the PK profile of natalizumab in stroke and multiple sclerosis (MS) patients. Studies 1801, 1802, 1803, 102, and 1805 refer to MS studies; study 201 refers to the ACTION study on acute ischemic stroke, as described herein. The data points indicate the mean+/−standard deviation of serum natalizumab concentration.

FIG. 9 is a table illustrating the scoring methodology for the Bartel Index.

FIG. 10 is a table illustrating the scoring methodology for NIHSS.

FIGS. 11A, 11B, and 11C are plots showing the relationship between AUC and weight (11A), baseline infarct volume (11B), and baseline NIHSS score (11C).

FIGS. 12A and 12B are graphs showing the relationship between Cmax and baseline NIHSS score (FIG. 12A) or between AUC and baseline NIHSS score (FIG. 12B).

FIG. 13 is a set of plots and table showing the Cmax and AUC (0-672 hours) of natalizumab in subjects binned by weight.

### DETAILED DESCRIPTION

The disclosure is based, at least in part, on the discovery that VLA-4 antagonists such as natalizumab can effectively improve clinical outcomes for victims of stroke, e.g., an ischemic stroke, e.g., an acute ischemic stroke, e.g., when administered within a specified time period after the last known normal (LKN). In some examples, the administration of a VLA-4 antagonist, e.g., natalizumab to a subject within a period of nine hours or less, e.g., 8, 7, 6 hours or less, after LKN, e.g., an ischemic stroke, e.g., acute ischemic stroke, provides an improvement in clinical outcomes, such as functional independence, activities of daily living, and/or cognitive ability. In some cases, the administration of a VLA-4 antagonist, e.g., natalizumab to a subject within a period of 12 hours or less (e.g., 12, 11, 10, 9 hours or less) after LKN may provide an improvement in clinical outcomes. In some cases, the administration of a VLA-4 antagonist, e.g., natalizumab, to the subject at a dosage of about 250 to 350, e.g., about 300, e.g., 300, mg; 350 to 500, e.g., 390 to 450, or 450+/−5%, or about 450, e.g., 450, mg; 550 to 650, e.g., 575 to 625, e.g., 600+/−5%, e.g., about 600, e.g., 600, mg, after the stroke provides an improvement in such clinical outcomes. Thus, treatment of stroke, e.g., acute ischemic stroke, with a VLA-4 antagonist, e.g., natalizumab, e.g., at a dosage of 250 to 650 mg, provides an extended time period for treating subjects having a stroke as compared to other approved treatments such as rtPA.

Embodiments of the invention are also based, at least in part, on the discovery that when administered a dosage of a VLA-4 antagonist, e.g., natalizumab, stroke patients vary in the exposure that they have to the drug. Results described herein show that patients having a higher exposure to the VLA-4 antagonist, e.g., natalizumab, tended to exhibit better clinical outcomes than those having a lower exposure to the drug. As described in the Example, exposure (e.g., as measured by area under the curve (AUC)) correlated with a number of factors including weight, baseline infarct volume, and baseline severity of stroke. Higher weight patients, patients with higher baseline infarct volumes, and patients with more severe strokes (higher NIHSS scores at baseline) tended to have a lower exposure to the VLA-4 antagonist, e.g., natalizumab. Analyses described herein indicate that AUC can be normalized by increasing the dosage. Accordingly, provided herein are compositions and methods that provide a dosage of VLA-4 antagonist, e.g., natalizumab-like antibody molecule (e.g., natalizumab), that ensures that a majority of stroke patients (e.g., patients with various weights; patients who have large infarct volumes at baseline; patients who have small infarct volumes at baseline; patients who have suffered a less than severe stroke (e.g., mild, or mild to moderate); as well as patients who have suffered a severe stroke) receive adequate coverage/exposure of the drug. It is believed that providing higher exposures to a larger proportion of stroke patients will result in better outcomes across the population, and especially in subpopulations with lower relative exposures, e.g., patients with higher body weight, higher baseline infarct volume, or severe stroke (as determined, e.g., by baseline NIHSS score). An exemplary dosage range of a VLA-4 antagonist, e.g., a natalizumab-like antibody molecule (e.g., natalizumab), includes 250 to 350, e.g., about 300, e.g., 300, mg; 350 to 500, e.g., 390 to 450, or 450+/−5%, or about 450, e.g., 450, mg; 550 to 650, e.g., 575 to 625, e.g., 600+/−5%, e.g., about 600, e.g., 600, mg. In embodiments, the dosage of VLA-4 antagonist, e.g., a natalizumab-like antibody molecule (e.g., natalizumab), is sufficient to achieve an AUC (e.g., over a period of time of 0-10 days, 0-20 days, 0-30 days, 0-60 days, 0-90 days, or 0-120 days) at least about 20,000 to 30,000, e.g., at least about 20,000, 25,000, or 30,000 mg*hr/L. In embodiments, the dosage of VLA-4 antagonist, e.g., a natalizumab-like antibody molecule (e.g., natalizumab), is sufficient to result in an AUC that is comparable to or higher than (e.g., within 5-10% of) the median AUC of a less than severe stroke patient or a patient having a lower body weight (e.g., of <80 kg).

In embodiments, the dosage can be tailored to the patient characteristics, e.g., severity of stroke, at baseline. For example, a patient having a more severe stroke is administered a higher dosage of the VLA-4 antagonist, e.g. a natalizumab-like antibody molecule (e.g., natalizumab), compared to a patient having a less severe stroke. An exemplary dosage range is 350 to 500 mg, e.g., 390-450 mg, e.g., about 450 mg, e.g., for a patient having a severe stroke. Another exemplary dosage range 250 to 390 mg, e.g., 275 to 325 mg, e.g., about 300 mg, e.g., for a patient having a less than severe stroke.

The invention is also based, at least in part, on the finding that a subgroup of stroke (e.g., ischemic stroke, e.g., acute ischemic stroke), patients that had a small to medium sized infarct at baseline (e.g., after stroke and before treatment) responded with a greater improvement in one or more clinical outcomes, e.g., a clinical outcome described herein, compared to patients having large infarcts at baseline. Accordingly, provided herein in some aspects are methods to treat a subject having a small to medium sized infarct at baseline. The compositions and methods described herein advantageously provide treatment to a greater number of patients than existing stroke therapies.

The following definitions are provided for specific terms used in the following written description and appended claims.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The terms “proteins” and “polypeptides” are used interchangeably herein.

“Baseline,” as used herein, refers to a value or measurement prior to administration of a therapy, e.g., a therapy described herein, e.g., a VLA-4 antagonist, e.g., a natalizumab-like antibody molecule (e.g., natalizumab).

The very late antigen (VLA) integrin superfamily is made up of functionally and structurally related glycoproteins consisting of heterodimeric (alpha and beta), transmembrane receptor molecules found in various combinations on almost every mammalian cell type. (For reviews see: E. C. Butcher, Cell, 67, 1033 (1991); D. Cox et al., “The Pharmacology of the Integrins.” Medicinal Research Rev. (1994) and V. W. Engleman et al., “Cell Adhesion Integrins as Pharmaceutical Targets” in Ann. Report in Medicinal Chemistry, Vol. 31, J. A. Bristol, Ed.; Acad. Press, N Y, 1996, p. 191). Integrins of the VLA family include (at present) VLA-1, -2, -3, -4, -5, -6, -9, and -11 in which each of the molecules comprise a β1 chain non-covalently bound to an α chain, (α1, α2, α3, α4, α5, α6 and the like), respectively.

The integrin alpha 4 beta 1 (α4β1) is a cell-surface receptor for fibronectin, VCAM-1, and possibly other ligands (the latter ligands individually and collectively referred to as “alpha4 ligand(s)”). The term α4β1 integrin (“VLA-4” or “a4b1” or “a4b1 integrin”, used interchangeably herein) refers to polypeptides which are capable of binding to VCAM-1 and members of the extracellular matrix proteins, most particularly fibronectin, or fragments thereof, although it will be appreciated by persons of ordinary skill in the art that other ligands for VLA-4 may exist and can be analyzed using conventional methods. Nevertheless, it is known that the alpha4 subunit will associate with other beta subunits besides beta1, therefore the term “alpha 4 integrin” or “alpha 4 subunit-containing integrin”, as used herein, refers to those integrins whose α4 subunit associates with one or another of the beta subunits. Another example of an “α4” integrin besides VLA4 is alpha4beta7 (α4β7) (See Lobb and Adams, supra).

In accordance with the methods described herein, provided herein are molecules that antagonize the action of more than one α4 subunit-containing integrin, e.g., small molecules or antibody molecules that antagonize both VLA-4 and α4β7 or other combinations of α4 subunit-containing integrins. Also provided herein are methods using a combination of molecules such that the combination antagonizes the action of more than one integrin, such as methods using several small molecules or antibody molecules that in combination antagonize both VLA-4 and α4β7 or other combinations of α4 subunit-containing integrins.

“Covalently coupled” means that the specified moieties (e.g., PEGylated VLA-4 antagonist, immunoglobulin fragment/VLA-4 antagonist) are either directly covalently bonded to one another, or else are indirectly covalently joined to one another through an intervening moiety or moieties, such as a spacer moiety or moieties. The intervening moiety or moieties are called a “coupling group”. The term “conjugated” is used interchangeably with “covalently coupled”. In this regard a “spacer” refers to a moiety that may be inserted between an amino acid or other component of a VLA-4 antagonist and the remainder of the molecule. A spacer may provide separation between the amino acid or other component and the rest of the molecule so as to prevent the modification from interfering with protein function and/or make it easier for the amino acid or other component to link with another moiety.

“Expression vector,” as used herein refers to a polynucleotide, such as a DNA plasmid or phage (among other common examples) which allows expression of at least one gene when the expression vector is introduced into a host cell. The vector may, or may not, be able to replicate in a cell.

“Functional equivalent” of an amino acid residue is (i) an amino acid having similar reactive properties as the amino acid residue that was replaced by the functional equivalent; (ii) an amino acid of an antagonist of the invention, the amino acid having similar properties as the amino acid residue that was replaced by the functional equivalent; (iii) a non-amino acid molecule having similar properties as the amino acid residue that was replaced by the functional equivalent.

A first polynucleotide encoding a proteinaceous antagonist of the invention is “functionally equivalent” compared with a second polynucleotide encoding the antagonist protein if it satisfies at least one of the following conditions:

(a): the “functional equivalent” is a first polynucleotide that hybridizes to the second polynucleotide under standard hybridization conditions and/or is degenerate to the first polynucleotide sequence. Most preferably, it encodes a mutant protein having the activity of a VLA-4 antagonist protein;

(b) the “functional equivalent” is a first polynucleotide that codes on expression for an amino acid sequence encoded by the second polynucleotide.

In accordance with the methods and compositions described herein, the VLA-4 antagonists include, but are not limited to, the agents listed herein as well as their functional equivalents. As used herein, the term “functional equivalent” therefore refers to a VLA-4 antagonist or a polynucleotide encoding the VLA-4 antagonist that has the same or an improved beneficial effect on the recipient as the VLA-4 antagonist of which it is deemed a functional equivalent. As will be appreciated by one of ordinary skill in the art, a functionally equivalent protein can be produced by recombinant techniques, e.g., by expressing a “functionally equivalent DNA”. Accordingly, the disclosure embraces integrin proteins encoded by naturally-occurring DNAs, as well as by non-naturally-occurring DNAs which encode the same protein as encoded by the naturally-occurring DNA. Due to the degeneracy of the nucleotide coding sequences, other polynucleotides may be used to encode integrin protein. These include all, or portions of the above sequences which are altered by the substitution of different codons that encode the same amino acid residue within the sequence, thus producing a silent change. Such altered sequences are regarded as equivalents of these sequences. For example, Phe (F) is coded for by two codons, TTC or TTT, Tyr (Y) is coded for by TAC or TAT and His (H) is coded for by CAC or CAT. On the other hand, Trp (W) is coded for by a single codon, TGG. Accordingly, it will be appreciated that for a given DNA sequence encoding a particular integrin there will be many DNA degenerate sequences that will code for it. These degenerate DNA sequences are considered within the scope of this disclosure.

The term “chimeric”, when referring to an antagonist, means that the antagonist is comprised of a linkage (chemical cross-linkage or covalent or other type) of two or more proteins having disparate structures and/or having disparate sources of origin. Thus, a chimeric VLA-4 antagonist may include one moiety that is a VLA-4 antagonist or fragment and another moiety that is not a VLA-4 antagonist.

A species of “chimeric” protein is a “fusion” or “fusion protein” which refers to a co-linear, covalent linkage of two or more proteins or fragments thereof via their individual peptide backbones, most preferably through genetic expression of a polynucleotide molecule encoding those proteins. Thus, preferred fusion proteins are chimeric proteins that include a VLA-4 antagonist or fragment covalently linked to a second moiety that is not a VLA-4 antagonist. Preferred fusion proteins include portions of intact antibodies that retain antigen-binding specificity, for example, Fab fragments, Fab′ fragments, F(ab′)2 fragments, F(v) fragments, heavy chain monomers or dimers, light chain monomers or dimers, dimers consisting of one heavy and one light chain, and the like.

In some embodiments, the other preferred fusion proteins are chimeric and comprise a VLA-4 antagonist moiety fused or otherwise linked to all or part of the hinge and constant regions of an immunoglobulin light chain, heavy chain, or both. Thus, the methods described herein can utilize a molecule that include: (1) an VLA-4 antagonist moiety, (2) a second peptide, e.g., one which increases solubility or in vivo life time of the VLA-4 antagonist moiety, e.g., a member of the immunoglobulin super family or fragment or portion thereof, e.g., a portion or a fragment of IgG, e.g., the human IgG1 heavy chain constant region, e.g., CH2, CH3, and hinge regions. Specifically, a “VLA-4 antagonist/lg fusion” is a protein comprising a biologically active VLA-4 antagonist (e.g. a soluble VLA-4 ligand), or a biologically active fragment thereof linked to an N-terminus of an immunoglobulin chain wherein a portion of the N-terminus of the immunoglobulin is replaced with the VLA-4 antagonist. A species of VLA-4 antagonist/lg fusion is a “VLA-4/Fc fusion” which is a protein comprising a VLA-4 antagonist, e.g., described herein, linked to at least a part of the constant domain of an immunoglobulin. A preferred Fc fusion comprises a VLA-4 antagonist, e.g., described herein, linked to a fragment of an antibody containing the C terminal domain of the heavy immunoglobulin chains.

The term “fusion protein” also means a VLA-4 antagonist chemically linked via a mono- or hetero-functional molecule to a second moiety that is not a VLA-4 antagonist (resulting in a “chimeric” molecule). Thus, one example of a chemically linked, as opposed to recombinantly linked, chimeric molecule that is a fusion protein may comprise: (1) VLA-4 subunit targeting moiety, e.g., a VCAM-1 moiety capable of binding to VLA-4) on the surface of VLA-4 bearing cells; (2) a second molecule which increases solubility or in vivo life time of the targeting moiety, e.g., a polyalkylene glycol polymer such as polyethylene glycol (PEG). The VLA-4 targeting moiety can be any naturally occurring VLA-4 ligand or fragment thereof, e.g., a VCAM-1 peptide or a similar conservatively substituted amino acid sequence.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

As used herein, the term “hybridizes under high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. High stringency hybridization conditions include hybridization in 6.times.SSC at about 45° C., followed by one or more washes in 0.2×SSC, 0.1% SDS at 65° C., or substantially similar conditions.

“Isolated” (used interchangeably with “substantially pure”), when applied to nucleic acid i.e., polynucleotide sequences that encode VLA antagonists, means an RNA or DNA polynucleotide, portion of genomic polynucleotide, cDNA or synthetic polynucleotide which, by virtue of its origin or manipulation: (i) is not associated with all of a polynucleotide with which it is associated in nature (e.g., is present in a host cell as an expression vector, or a portion thereof); or (ii) is linked to a nucleic acid or other chemical moiety other than that to which it is linked in nature; or (iii) does not occur in nature. By “isolated” it is further meant a polynucleotide sequence that is: (i) amplified in vitro by, for example, polymerase chain reaction (PCR); (ii) synthesized chemically; (iii) produced recombinantly by cloning; or (iv) purified, as by cleavage and gel separation. Thus, “substantially pure nucleic acid” is a nucleic acid which is not immediately contiguous with one or both of the coding sequences with which it is normally contiguous in the naturally occurring genome of the organism from which the nucleic acid is derived. Substantially pure DNA also includes a recombinant DNA which is part of a hybrid gene encoding additional integrin sequences.

“Isolated” (used interchangeably with “substantially pure”), when applied to polypeptides means a polypeptide or a portion thereof which, by virtue of its origin or manipulation: (i) is present in a host cell as the expression product of a portion of an expression vector; or (ii) is linked to a protein or other chemical moiety other than that to which it is linked in nature; or (iii) does not occur in nature, for example, a protein that is chemically manipulated by appending, or adding at least one hydrophobic moiety to the protein so that the protein is in a form not found in nature. By “isolated” it is further meant a protein that is: (i) synthesized chemically; or (ii) expressed in a host cell and purified away from associated and contaminating proteins. The term generally means a polypeptide that has been separated from other proteins and nucleic acids with which it naturally occurs. Preferably, the polypeptide is also separated from substances such as antibodies or gel matrices (polyacrylamide) which are used to purify it.

A “pharmacological agent” is defined as one or more compounds or molecules or other chemical entities administered to a subject (in addition to the VLA-4 antagonists) that affects the action of the antagonist. The term “pharmacological agent” as used herein refers to such an agent(s) that are administered during “combination therapy” where the VLA-4 antagonist is administered either prior to, after, or simultaneously with, administration of one or more pharmacological agents.

“Protein,” as used herein refers to any polymer consisting essentially of any of the 20 amino acids. Although “polypeptide” is often used in reference to relatively large polypeptides, and “peptide” is often used in reference to small polypeptides, usage of these terms in the art overlaps and is varied. The term “protein” as used herein refers to peptides, proteins and polypeptides, unless otherwise noted.

The terms “peptide(s)”, “protein(s)” and “polypeptide(s)” are used interchangeably herein. The terms “polynucleotide sequence” and “nucleotide sequence” are also used interchangeably herein.

“Recombinant,” as used herein, means that a protein is derived from recombinant, mammalian expression systems. Since integrin is not glycosylated nor contains disulfide bonds, it can be expressed in most prokaryotic and eukaryotic expression systems.

“Small molecule” VLA-4 antagonist refers to chemical agents (i.e., organic molecules) capable of disrupting the integrin/integrin ligand interaction by, for instance, blocking VLA-4/VCAM interactions by binding VLA-4 on the surface of cells or binding VCAM-1 on the surface of cells. Such small molecules may also bind respective VLA-4 and VCAM-1 receptors. VLA-4 and VCAM-1 small molecule inhibitors may themselves be peptides, semi-peptidic compounds or non-peptidic compounds, such as small organic molecules that are antagonists of the VCAM-1/VLA-4 interaction.

A VLA-4 antagonist (and a therapeutic composition comprising the same) is said to have “therapeutic efficacy,” and an amount of the agent is said to be “therapeutically effective,” if administration of that amount of the agent is sufficient to cause a clinically significant improvement in neurological recovery in a standard neurological test (see below, Methods of Treatment) when administered to a subject (e.g., an animal model or human patient) after brain damage (e.g., stroke, e.g., ischemic stroke, e.g., acute ischemic stroke).

The term “treating”, as used herein, refers to administering a therapy in an amount, manner (e.g., schedule of administration), and/or mode (e.g., route of administration), effective to improve a disorder or a symptom thereof, or to prevent or slow the progression of a disorder or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

The term “biologic” refers to a protein-based therapeutic agent. In a preferred embodiment, the biologic is at least 10, 20, 130, 40, 50 or 100 amino acid residues in length.

A “VLA-4 binding agent” refers to any compound that binds to VLA-4 integrin with a K_{d} of less than 10⁻⁶ M. An example of a VLA-4 binding agent is a VLA-4 binding protein, e.g., a VLA-4 binding antibody such as a natalizumab-like antibody molecule (e.g., natalizumab).

A “VLA-4 antagonist” refers to any compound that at least partially inhibits an activity of a VLA-4 integrin, particularly a binding activity of a VLA-4 integrin or a signaling activity, e.g., ability to transduce a VLA-4 mediated signal. For example, a VLA-4 antagonist may inhibit binding of VLA-4 to a cognate ligand of VLA-4, e.g., a cell surface protein such as VCAM-1, or to an extracellular matrix component, such as fibronectin or osteopontin. A typical VLA-4 antagonist can bind to VLA-4 or to a VLA-4 ligand, e.g., VCAM-1 or an extracellular matrix component, such as fibronectin or osteopontin. A VLA-4 antagonist that binds to VLA-4 may bind to either the α4 subunit or the (31 subunit, or to both. A VLA-4 antagonist may also interact with other α4 subunit containing integrins (e.g., α4β7) or with other (31 containing integrins. A VLA-4 antagonist may bind to VLA-4 or to a VLA-4 ligand with a K_{d} of less than 10⁻⁶, 10⁻⁷, 10⁻⁸, 10⁻⁹, or 10⁻¹⁰ M.

The term “antibody molecule” refers to an antibody or antigen binding fragment thereof. As used herein, the term “antibody” refers to a protein that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, e.g., intact immunoglobulins of types IgA, IgG, IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A., et al. (1991) Sequences of Proteins of Immunological Interest, Fifth Edition, US Department of Health and Human Services, NIH Publication No. 91-3242; and Chothia, C. et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay 1988 Ann. Rev Immunol. 6:381-405).

As used herein, an “immunoglobulin variable domain sequence” refers to an amino acid sequence that can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with VLA-4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH₁, CH₂ and CH₃. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

In certain embodiments, one or more regions of an antibody can be human, effectively human, or humanized. in some examples, one or more of the variable regions can be human or effectively human. For example, one or more of the CDRs, e.g., HC CDR1, HC CDR2, HC CDR3, LC CDR1, LC CDR2, and LC CDR3, can be human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions.

In some embodiments all or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antigen-binding fragment” of a full length antibody refers to one or more fragments of a full-length antibody that retain the ability to specifically bind to a target of interest, e.g., VLA-4. Examples of binding fragments encompassed within the term “antigen-binding fragment” of a full length antibody include (i) a Fab fragment, a monovalent fragment consisting of the VL, VH, CL and CH1 domains; (ii) a F(ab′).sub.2 fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:54-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See, e.g., Bird et al. (1988) Science 242:423-426; and Huston et al. (1988) Proc. Natl. Acad. Sci. USA 85:5879-5883.

A “fixed dose” or dosage unit form refers to physically discrete units suited as unitary doses for a subject to be treated. Each unit contains a predetermined quantity of active compound calculated to produce the desired therapeutic effect in association with the required pharmaceutical carrier.

A “dosage” as used herein refers to a quantity or amount of a therapeutic agent. Typically a dosage is the amount administered to the subject in a single administration, e.g., in a single injection, a single infusion, or single administration of one or more oral fixed doses. In an embodiment, an exemplary dosage is 450+/−5% mgs. In an embodiment, an exemplary dosageis 600+/−5% mgs.

The terms “therapeutically effective dosage” and “therapeutically effective amount” refer to that amount of an agent (e.g., an agent described herein, e.g., VLA-4 antagonist, e.g., a natalizumab-like antibody molecule (e.g., natalizumab)) which results in at least one of prevention or delay of onset or amelioration of symptoms of a neurological disorder, e.g., stroke ischemic stroke, e.g., acute ischemic stroke, or hemorrhagic stroke, e.g., intracerebral hemorrhage), subarachnoid hemorrhage, or traumatic brain injury, in a subject or an attainment of a desired biological outcome, such as reduced size of infarct or reduced disability/increased functional independence. A therapeutically effective amount of an agent can vary according to factors such as disease state, age, sex, weight of the subject, as well as the ability of the agent to elicit a desired responses in the subject. In embodiments, a therapeutically effective amount is one in which the therapeutically beneficial effects of the agent outweight its toxic or adverse effects.

“Cmax” refers to the maximum plasma concentration of a drug (e.g., a drug described herein) achieved after a single administration of a dosage form of the drug as described herein.

“Area under the curve” or “AUC” refers to the area under a curve representing the concentration of a compound/molecule or metabolite thereof in a biological fluid, e.g., plasma and blood, in a patient as a function of time (e.g., 0-10 days, 0-20 days, 0-30 days, 0-60 days, 0-90 days, or 0-120 days) following administration of the compound/molecule to the patient. Suitable methods for calculating the AUC from a drug concentration-versus-time curve are known in the art.

“Natalizumab” is an exemplary VLA-4 antagonist that is described in greater detail below.

A “natalizumab-like antibody molecule” as used herein is natalizumab or an antibody molecule that differs from the amino acid sequence of natalizumab at least one amino acid residue, but which has similar biological properties to natalizumab. In an embodiment it comprises:

a)i) light chain CDR1, CDR2 and CDR3, e.g., Chothia or Kabat light chain CDRs, from natalizumab;

a)ii) light chain CDRs CDR1, CDR2 and CDR3, that collectively, differ by no more than 1, 2, 3, 4, 5, or 6 amino acid residues from the light chain CDRs of a)i);

a)iii) a light chain variable region of natalizumab;

a)iv) an antigen binding fragment of a)iii);

a)v) a sequence that differs by no more than 1, 2, 3, 4, 5, 6, 7, 8, 9, or 10, residues from the sequence of a)iii) or a)iv); and

b)i) heavy chain CDR1, CDR2 and CDR3, e.g., Chothia or Kabat heavy chain CDRs, from natalizumab;

b)ii) heavy chain CDRs CDR1, CDR2 and CDR3, that collectively, differ by no more than 1, 2, 3, 4, 5, or 6 amino acid residues from the heavy chain CDRs of b)i);

b)iii) a heavy chain variable region of natalizumab;

b)iv) an antigen binding fragment of b)iii);

b)v) a sequence that differs by no more than 1, 2, 3, 4, 5, 6, 7, 8, 9, or 10, residues from the sequence of b)iii) or b)iv).

In an embodiment the natalizumab-like antibody molecule comprises a)i) and b)i).

In an embodiment natalizumab-like antibody molecule comprises a)ii) and b)ii).

In an embodiment natalizumab-like antibody molecule comprises a)iii) and b)iii).

In an embodiment the natalizumab-like antibody molecule comprises a)iv) and b)iv).

In an embodiment the natalizumab-like antibody molecule comprises a)v) and b)v).

In an embodiments, the natalizumab-like antibody molecule is an antibody molecule i) comprising one or more CDRs, e.g., all three HC CDRs and/or all three LC CDRs of natalizumab, ii) comprising one or more CDRs that are, in sum, at least 80, 85, 90, 92, 94, 95, 96, 97, 98, 99%, or 100% identical to natalizumab or iii) comprising a HC and/or LC variable domain amino acid sequence having at least 70, 80, 85, 90, 92, 95, 97, 98, 99, or 100% identity to the amino acid sequence of the HC and/or LC variable domain of natalizumab.

Severity of a stroke can be determined by evaluating a patient who has suffered a stroke, e.g., at baseline, e.g., after stroke onset and prior to treatment, by using one or more of the evaluations described herein, e.g., NIHSS score. For example, a “severe stroke” or a “less than severe stroke” can be indicated by a number of ways.

“Severe stroke” as used herein refers to a stroke having a NIHSS score equal to or greater than 15, e.g., equal to or greater than 21, e.g., in the range of 15 to 24 or 21 to 42. In an embodiment the NIHSS (or other) evaluation is made at baseline, e.g., after stroke onset and prior to treatment with natalizumab or a natalizumab-like antibody molecule. Other forms of evaluation can be used to determine that a stroke is a severe stroke and if it is equivalent in severity of a stroke having a NIHSS score equal to or greater than 15, e.g., equal to or greater than 21, e.g., in the range of 15 to 24 or 21 to 42. In some embodiments, a severe stroke can include a very severe stroke (e.g., having a NIHSS score greater than 25). See, e.g., Brott et al. Stroke. 20(1989):864-70.

“Less than severe stroke” as used herein refers to a stroke having a NIHSS score of less than 15. In an embodiment the NIHSS (or other) evaluation is made at baseline, e.g., after stroke onset and prior to treatment with natalizumab or a natalizumab-like antibody molecule. Less than severe strokes can be further divided into several categories. Other forms of evaluation can be used to determine that a stroke is a less than severe stroke and if it is equivalent in severity of a stroke having a NIHSS score of less than 15. For example, a NIHSS of 1-5 is indicative of mild stroke; and a score of 5-14 is indicative of mild to moderate stroke. See, e.g., Brott et al. Stroke. 20(1989):864-70.

“Last known normal” or “LKN” as used herein refers to the time at which a subject was last known to be normal, e.g., exhibit no signs or symptoms of a disease described herein, e.g., stroke, e.g., ischemic stroke (e.g., acute ischemic stroke), or a hemorrhagic stroke (e.g., intracerebral hemorrhage), or a subarachnoid hemorrhage, or a traumatic brain injury (TBI). The LKN time can be determined by the subject or by another individual (e.g., family member, e.g., father, mother, child, spouse, relative; or neighbor) who has observed the subject prior to (e.g., within 6 hours, e.g., 6, 5, 4, 3, 2, 1 hour or less, and/or during the beginning of a symptom of the disease. The LKN can be self-reported by the subject, reported by a witness, or both.

Practice of the present invention will employ, unless indicated otherwise, conventional techniques of cell biology, cell culture, molecular biology, microbiology, recombinant DNA, protein chemistry, pharmacology and immunology, which are within the skill of the art. Such techniques are described in the literature. Unless stipulated otherwise, all references cited in the Detailed Description are incorporated herein by reference.

### VLA-4 Antagonists

A VLA-4 antagonist is an antagonist of interactions of α4 integrins with their ligands, such as the VCAM-1/VLA-4 interaction. This is an agent, e.g., a polypeptide or other molecule, which can inhibit or block VCAM-1 and/or VLA-4-mediated binding or which can otherwise modulate VCAM-1 and/or VLA-4 function, e.g., by inhibiting or blocking VLA-4-ligand mediated VLA-4 signal transduction or VCAM-1-ligand mediated VCAM-1 signal transduction and which is effective in the treatment of acute brain injury, preferably in the same manner as anti-VLA-4 binding agents such as anti-VLA-4 antibodies.

A VLA-4 antagonist can have one or more of the following properties: (1) it coats, or binds to, VLA-4 on the surface of a VLA-4 bearing cell (e.g., an endothelial cell) with sufficient specificity to inhibit a VLA-4-ligand/VLA-4 interaction, e.g., the VCAM-1/VLA-4 interaction; (2) it coats, or binds to, VLA-4 on the surface of a VLA-4 bearing cell (i.e., a lymphocyte) with sufficient specificity to modify, and preferably to inhibit, transduction of a VLA-4-mediated signal e.g., VLA-4/VCAM-1-mediated signaling; (3) it coats, or binds to, a VLA-4-ligand, (e.g., VCAM-1) on endothelial cells with sufficient specificity to inhibit the VLA-4/VCAM-1 interaction; (4) it coats, or binds to, a VLA-4-ligand (e.g., VCAM-1) with sufficient specificity to modify, and preferably to inhibit, transduction of VLA-4-ligand mediated VLA-4 signaling, e.g., VCAM-1-mediated VLA-4 signaling. In preferred embodiments the antagonist has one or both of properties 1 and 2. In other preferred embodiments the antagonist has one or both of properties 3 and 4. Moreover, more than one antagonist can be administered to a patient, e.g., an agent which binds to VLA-4 can be combined with an agent which binds to VCAM-1.

For example, antibody molecules as well as soluble forms of the natural binding proteins for VLA-4 and VCAM-1 are useful.

VLA-4 Antagonist Antibody Molecules

Natalizumab, an α4 integrin binding antibody, inhibits the migration of leukocytes from the blood to the central nervous system. Natalizumab binds to VLA-4 on the surface of activated T-cells and other mononuclear leukocytes. It can disrupt adhesion between the T-cell and endothelial cells, and thus prevent migration of mononuclear leukocytes across the endothelium and into the parenchyma. As a result, the levels of proinflammatory cytokines can also be reduced.

Natalizumab and related VLA-4 binding antibodies are described, e.g., in U.S. Pat. No. 5,840,299. Monoclonal antibodies 21.6 and HP1/2 are exemplary murine monoclonal antibodies that bind VLA-4. Natalizumab is a humanized version of murine monoclonal antibody 21.6 (see, e.g., U.S. Pat. No. 5,840,299). A humanized version of HP1/2 has also been described (see, e.g., U.S. Pat. No. 6,602,503). Several additional VLA-4 binding monoclonal antibodies, such as HP2/1, HP2/4, L25 and P4C2, are described, e.g., in U.S. Pat. No. 6,602,503; Sanchez-Madrid et al., 1986 Eur. J. Immunol., 16:1343-1349; Hemler et al., 1987 J. Biol. Chem. 2:11478-11485; Issekutz and Wykretowicz, 1991, J. Immunol., 147: 109 (TA-2 mab); Pulido et al., 1991 J. Biol. Chem., 266(16):10241-10245; and U.S. Pat. No. 5,888,507.

In embodiments, the VLA-4 antagonist comprises an antibody molecule that comprises CDR1, CDR2 and CDR3 from the light chain and CDR1, CDR2 and CDR3 from the heavy chain of natalizumab. In embodiments, the VLA-4 antagonist comprises an antibody molecule that comprises the light chain variable region and the heavy chain variable region of natalizumab.

In embodiments, a natalizumab-like antibody molecule, e.g., natalizumab, comprises a light chain amino acid sequence, variable light chain amino acid sequence, and/or one or more (e.g., one or more, two or more, or all three) light chain CDR sequences described herein, e.g., of natalizumab. In embodiments, a natalizumab-like antibody molecule, e.g., natalizumab, comprises a light chain comprising a CDR1 (SEQ ID NO: 3), CDR2 (SEQ ID NO: 4) and CDR3 (SEQ ID NO: 5) and a heavy chain comprising a CDR1 (SEQ ID NO: 8), CDR2 (SEQ ID NO: 9) and CDR3 (SEQ ID NO: 10). In embodiments, a natalizumab-like antibody molecule, e.g., natalizumab, comprises a light chain variable region sequence of SEQ ID NO: 2 and a heavy chain variable region sequence of SEQ ID NO: 7.

In embodiments, a natalizumab-like antibody molecule, e.g., natalizumab, comprises one or more (e.g., one or more, two or more, or all three) CDRs (e.g., according to a numbering scheme described herein, e.g., Kabat and/or Chothia) from a variable light chain sequence of SEQ ID NO: 2. In embodiments, a natalizumab-like antibody molecule, e.g., natalizumab, comprises one or more (e.g., one or more, two or more, or all three) CDRs (e.g., according to a numbering scheme described herein, e.g., Kabat and/or Chothia) from a variable heavy chain sequence of SEQ ID NO: 7.

Natalizumab light chain amino acid sequence (SEQ ID NO: 1) (the CDRs (according to the Kabat numbering scheme) are boxed and the variable light (VL) chain sequence is underlined):

(SEQ ID NO: 1)

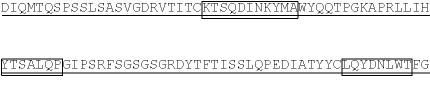

QGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

Natalizumab VL sequence (SEQ ID NO: 2) (the CDRs (according to the Kabat numbering scheme) are boxed):

(SEQ ID NO: 2)

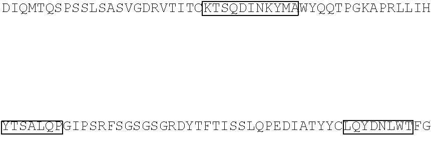

QGTKVEIK

Natalizumab light chain CDRs (LCDR) according to the Kabat numbering scheme (Kabat et al. (1991), “Sequences of Proteins of Immunological Interest,” 5th Ed. Public Health Service, National Institutes of Health, Bethesda, Md.) are as follows:

LCDR1:
(SEQ ID NO: 3)
KTSQDINKYMA
LCDR2:
(SEQ ID NO: 4)
YTSALQP
LCDR3:
(SEQ ID NO: 5)
LQYDNLWT

In embodiments, a natalizumab-like antibody molecule, e.g., natalizumab, comprises a heavy chain amino acid sequence, variable heavy chain amino acid sequence, and/or one or more (e.g., one or more, two or more, or all three) heavy chain CDR sequences described herein, e.g., shown below.

Natalizumab heavy chain amino acid sequence, where Q¹ can be a glutamine or a pyroglutamic acid and where K² can be a lysine or no amino acid residue (SEQ ID NO: 6) (the CDRs (according to the Kabat numbering scheme) are boxed; the variable heavy chain (VH) sequence is underlined):

(SEQ ID NO: 6)

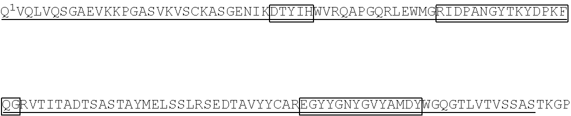

SVFPLAPCSRSTSESTAALGCLVKDYFPEPVTVSWNSGALTSGVHTFPAVLQSSGLYSLSSVVTV
PSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDIAVEWESNGQ
PENNYKTIPPVLDSDGSFFLYSRLIVDKSRWQEGNVESCSVMHEALHNHYTQKSLSLSLGK²

Natalizumab VH sequence, where Q¹ can be a glutamine or a glutamine cyclized to pyroglutamic acid (SEQ ID NO: 7) (the CDRs (according to the Kabat numbering scheme) are boxed):

(SEQ ID NO: 7)

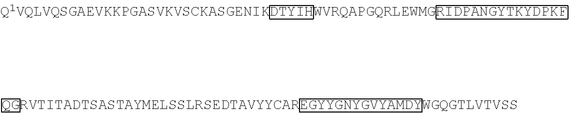

Natalizumab heavy chain CDRs (HCDR) according to the Kabat numbering scheme (Kabat et al. (1991), “Sequences of Proteins of Immunological Interest,” 5th Ed. Public Health Service, National Institutes of Health, Bethesda, Md.) are as follows:

HCDR1:
(SEQ ID NO: 8)
DTYIH
HCDR2:
(SEQ ID NO: 9)
RIDPANGYTKYDPKFQG
HCDR3:
(SEQ ID NO: 10)
EGYYGNYGVYAMDY

Some VLA-4 binding antibody molecules recognize epitopes of the α4 subunit that are involved in binding to a cognate ligand, e.g., VCAM-1 or fibronectin. Many such antibody molecules inhibit binding of VLA-4 to cognate ligands (e.g., VCAM-1 and fibronectin).

Some useful VLA-4 binding antibodies can interact with VLA-4 on cells, e.g., lymphocytes, but do not cause cell aggregation. However, other VLA-4 binding antibodies have been observed to cause such aggregation. HP1/2 does not cause cell aggregation. The HP1/2 monoclonal antibody (Sanchez-Madrid et al., 1986) has an extremely high potency, blocks VLA-4 interaction with both VCAM1 and fibronectin, and has the specificity for epitope B on VLA-4. This antibody and other B epitope-specific antibodies (such as B1 or B2 epitope binding antibodies; Pulido et al., 1991, supra) represent one class of VLA-4 binding antibodies that can be used in the methods described herein. Antibodies that compete for binding with a VLA-4 binding antibody, e.g., natalizumab, can also be used in the methods described herein.

An exemplary VLA-4 binding antibody molecule has one or more CDRs, e.g., all three HC CDRs and/or all three LC CDRs of a particular antibody disclosed herein, or CDRs that are, in sum, at least 80, 85, 90, 92, 94, 95, 96, 97, 98, 99% identical to such an antibody, e.g., natalizumab. In one embodiment, the H1 and H2 hypervariable loops have the same canonical structure as those of an antibody described herein. In one embodiment, the L1 and L2 hypervariable loops have the same canonical structure as those of an antibody molecule described herein.

In one embodiment, the amino acid sequence of the HC and/or LC variable domain sequence is at least 70, 80, 85, 90, 92, 95, 97, 98, 99, or 100% identical to the amino acid sequence of the HC and/or LC variable domain of an antibody described herein, e.g., natalizumab. The amino acid sequence of the HC and/or LC variable domain sequence can differ by at least one amino acid, but no more than ten, eight, six, five, four, three, or two amino acids from the corresponding sequence of an antibody described herein, e.g., natalizumab. For example, the differences may be primarily or entirely in the framework regions.

The amino acid sequences of the HC and LC variable domain sequences can be encoded by a nucleic acid sequence that hybridizes under high stringency conditions to a nucleic acid sequence described herein or one that encodes a variable domain or an amino acid sequence described herein. In one embodiment, the amino acid sequences of one or more framework regions (e.g., FR1, FR2, FR3, and/or FR4) of the HC and/or LC variable domain are at least 70, 80, 85, 90, 92, 95, 97, 98, 99, or 100% identical to corresponding framework regions of the HC and LC variable domains of an antibody described herein. In one embodiment, one or more heavy or light chain framework regions (e.g., HC FR1, FR2, and FR3) are at least 70, 80, 85, 90, 95, 96, 97, 98, or 100% identical to the sequence of corresponding framework regions from a human germline antibody.

Other VLA-4 Antagonist Polypeptides

In some embodiments, the VLA-4 antagonist can be a soluble form of a ligand. Soluble forms of the ligand proteins include soluble VCAM-I or fibronectin peptides, VCAM-I fusion proteins, or bifunctional VCAM-I/Ig fusion proteins. For example, a soluble form of a VLA-4 ligand or a fragment thereof may be administered to bind to VLA-4, and in some instances, compete for a VLA-4 binding site on cells, thereby leading to effects similar to the administration of antagonists such as anti-VLA-4 antibodies. For example, soluble VLA-4 integrin mutants that bind VLA-4 ligand but do not elicit integrin-dependent signaling are suitable for use in the described methods. Such mutants can act as competitive inhibitors of wild type integrin protein and are considered “antagonists.” Soluble forms of the natural binding proteins for VLA-4 include soluble VCAM-1 peptides, VCAM-1 fusion proteins, bifunctional VCAM-1/lg fusion proteins (e.g. “chimeric” molecules, discussed above), fibronectin, fibronectin having an alternatively spliced non-type III connecting segment, and fibronectin peptides containing the amino acid sequence EILDV or a similar conservatively substituted amino acid sequence. As used herein, a “soluble VLA-4 peptide” or a “soluble VCAM-1 peptide” is an VLA4 or VCAM-1 polypeptide incapable of anchoring itself in a membrane. Such soluble polypeptides include, for example, VLA-4 and VCAM polypeptides that lack a sufficient portion of their membrane spanning domain to anchor the polypeptide or are modified such that the membrane spanning domain is non-functional. These binding agents can act by competing with the cell-surface binding protein for VLA-4 or by otherwise altering VLA-4 function. For example, a soluble form of VCAM-1 (see, e.g., Osborn et al. 1989, Cell, 59: 1203-1211) or a fragment thereof may be administered to bind to VLA-4, and preferably compete for a VLA-4 binding site on VCAM-1-bearing cells, thereby leading to effects similar to the administration of antagonists such as small molecules or anti-VLA-4 antibodies.

Small Molecule VLA-4 Antagonists

“Small molecules” are agents that mimic the action of peptides to disrupt VLA-4/ligand interactions by, for instance, binding VLA-4 and blocking interaction with a VLA-4 ligand (e.g., VCAM-I or fibronectin), or by binding a VLA-4 ligand and preventing the ligand from interacting with VLA-4. One exemplary small molecule is an oligosaccharide that mimics the binding domain of a VLA-4 ligand (e.g., fibronectin or VCAM-I) and binds the ligand-binding domain of VLA-4. (See, Devlin et al., Science 249: 400-406 (1990); Scott and Smith, Science 249:386-390 (1990); and U.S. Pat. No. 4,833,092 (Geysen), all incorporated herein by reference.)

A “small molecule” may be chemical compound, e.g., an organic compound, or a small peptide, or a larger peptide-containing organic compound or non-peptidic organic compound. A “small molecule” is not intended to encompass an antibody or antibody fragment. Although the molecular weight of small molecules is generally less than 2000 Daltons, this figure is not intended as an absolute upper limit on molecular weight.

Examples of other small molecules useful in the invention can be found in Komoriya et al. (“The Minimal Essential Sequence for a Major Cell Type-Specific Adhesion Site (CS1) Within the Alternatively Spliced Type III Connecting Segment Domain of Fibronectin Is Leucine-Aspartic Acid-Valine”, J. Biol. Chem., 266 (23), pp. 15075-79 (1991)). They identified the minimum active amino acid sequence necessary to bind VLA-4 and synthesized a variety of overlapping peptides based on the amino acid sequence of the CS-1 region (the VLA-4 binding domain) of a particular species of fibronectin. They identified an 8-amino acid peptide, Glu-Ile-Leu-Asp-Val-Pro-Ser-Thr, as well as two smaller overlapping pentapeptides, Glu-Ile-Leu-Asp-Val and Leu-Asp-Val-Pro-Ser, that possessed inhibitory activity against fibronectin-dependent cell adhesion. Certain larger peptides containing the LDV sequence were subsequently shown to be active in vivo (T. A. Ferguson et al., “Two integrin Binding Peptides Abrogate T-cell-Mediated Immune Responses In Vivo”, Proc. Natl. Acad. Sci. USA, 88, pp. 8072-76 (1991); and S. M. Wahl et al., “Synthetic Fibronectin Peptides Suppress Arthritis in Rats by Interrupting Leukocyte Adhesion and Recruitment”, J. Clin. Invest., 94, pp. 655-62 (1994)). A cyclic pentapeptide, Arg-Cys-Asp-TPro-Cys (wherein TPro denotes 4-thioproline), which can inhibit both VLA-4 and VLA-5 adhesion to fibronectin has also been described. (See, e.g., D. M. Nowlin et al. “A Novel Cyclic Pentapeptide Inhibits Alpha4Beta1 Integrin-mediated Cell Adhesion”, J. Biol. Chem., 268(27), pp. 20352-59 (1993); and PCT publication PCT/US91/04862). This pentapeptide was based on the tripeptide sequence Arg-Gly-Asp from fibronectin which had been known as a common motif in the recognition site for several extracellular-matrix proteins. Examples of other VLA-4 inhibitors have been reported, for example, in Adams et al. “Cell Adhesion Inhibitors”, PCT US97/13013, describing linear peptidyl compounds containing beta-amino acids which have cell adhesion inhibitory activity. International patent applications WO 94/15958 and WO 92/00995 describe cyclic peptide and peptidomimetic compounds with cell adhesion inhibitory activity. International patent applications WO 93/08823 and WO 92/08464 describe guanidinyl-, urea- and thiourea-containing cell adhesion inhibitory compounds. U.S. Pat. No. 5,260,277 describes guanidinyl cell adhesion modulation compounds. Other peptidyl antagonists of VLA-4 have been described in D. Y. Jackson et al., “Potent α4β1 peptide antagonists as potential anti-inflammatory agents”, J. Med. Chem., 40,3359 (1997); H. Shroff et al., “Small peptide inhibitors of a4f37 mediated MadCAM-1 adhesion to lymphocytes”, Bio. Med, Chem. Lett., 1 2495 (1996); U.S. Pat. No. 5,510,332, PCT Publications WO 98/53814, WO97/03094, WO97/02289, WO96/40781, WO96/22966, WO96/20216, WO96/01644, WO96106108, and WO95/15973, and others.

Such small molecule agents may be produced by synthesizing a plurality of peptides (e.g., 5 to 20 amino acids in length), semi-peptidic compounds or non-peptidic, organic compounds, and then screening those compounds for their ability to inhibit the VLA-4/VCAM interaction. See generally U.S. Pat. No. 4,833,092, Scott and Smith, “Searching for Peptide Ligands with an Epitope Library”, Science, 249, pp. 386-90 (1990), and Devlin et al., “Random Peptide Libraries: A Source of Specific Protein Binding Molecules”, Science, 249, pp. 40407 (1990).

### Antibody Generation

Antibodies that bind to VLA-4 can be generated by immunization, e.g., using an animal, or by in vitro methods such as phage display. All or part of VLA-4 can be used as an immunogen. For example, the extracellular region of the α4 subunit can be used as an immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XenoMouse™, Green et al., Nature Genetics 7:13-21 (1994), US 2003-0070185, U.S. Pat. No. 5,789,650, and WO 96/34096.

Non-human antibodies to VLA-4 can also be produced, e.g., in a rodent. The non-human antibody can be humanized, e.g., as described in U.S. Pat. No. 6,602,503, EP 239 400, U.S. Pat. Nos. 5,693,761, and 6,407,213.

EP 239 400 (Winter et al.) describes altering antibodies by substitution (within a given variable region) of their complementarity determining regions (CDRs) for one species with those from another. CDR-substituted antibodies can be less likely to elicit an immune response in humans compared to true chimeric antibodies because the CDR-substituted antibodies contain considerably less non-human components (Riechmann et al., 1988, Nature 332, 323-327; Verhoeyen et al., 1988, Science 239, 1534-1536). Typically, CDRs of a murine antibody substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody.

Queen et al. (Proc. Natl. Acad. Sci. U.S.A. 86:10029-33, 1989) and WO 90/07861 have described a process that includes choosing human V framework regions by computer analysis for optimal protein sequence homology to the V region framework of the original murine antibody, and modeling the tertiary structure of the murine V region to visualize framework amino acid residues that are likely to interact with the murine CDRs. These murine amino acid residues are then superimposed on the homologous human framework. See also U.S. Pat. Nos. 5,693,762; 5,693,761; 5,585,089; and U.S. Pat. No. 5,530,101. Tempest et al., 1991, Biotechnology 9:266-271, utilize, as standard, the V region frameworks derived from NEWM and REI heavy and light chains, respectively, for CDR-grafting without radical introduction of mouse residues. An advantage of using the Tempest et al. approach to construct NEWM and REI based humanized antibodies is that the three dimensional structures of NEWM and REI variable regions are known from x-ray crystallography and thus specific interactions between CDRs and V region framework residues can be modeled.

Non-human antibodies can be modified to include substitutions that insert human immunoglobulin sequences, e.g., consensus human amino acid residues at particular positions, e.g., at one or more (preferably at least five, ten, twelve, or all) of the following positions: (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213.

Fully human monoclonal antibodies that bind to VLA-4 can be produced, e.g., using in vitro-primed human splenocytes, as described by Boerner et al., 1991, J. Immunol., 147, 86-95. They may be prepared by repertoire cloning as described by Persson et al., 1991, Proc. Nat. Acad. Sci. USA, 88: 2432-2436 or by Huang and Stollar, 1991, J. Immunol. Methods 141, 227-236; also U.S. Pat. No. 5,798,230. Large nonimmunized human phage display libraries may also be used to isolate high affinity antibodies that can be developed as human therapeutics using standard phage technology (see, e.g., Vaughan et al, 1996; Hoogenboom et al. (1998) Immunotechnology 4:1-20; and Hoogenboom et al. (2000) Immunol Today 2:371-8; US 2003-0232333). Transgenic animals, e.g., transgenic mice, expressing human antibody gene sequences may be used to produce human monoclonal antibodies using technology as described in, e.g., Lonberg N. (2005) Nat. Biotechnol. 23(9):1117-25.

Antibody Production

Antibodies can be produced in prokaryotic and eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one embodiment, antibodies, particularly full length antibodies, e.g., IgG's, are produced in mammalian cells. Exemplary mammalian host cells for recombinant expression include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NSO myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). Exemplary selectable marker genes include the dihydrofolate reductase (DHFR) gene (for use in dhfr⁻ host cells with methotrexate selection/amplification) and the neo gene (for G418 selection).

In an exemplary system for recombinant expression of an antibody (e.g., a fall length antibody or an antigen-binding portion thereof), a recombinant expression vector encoding both the antibody heavy chain and the antibody light chain is introduced into dhfr⁻CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies may also include modifications, e.g., modifications that alter Fc function, e.g., to decrease or remove interaction with an Fc receptor or with C1q, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some antibodies that include an Fc domain, the antibody production system may be designed to synthesize antibodies in which the Fc region is glycosylated. For example, the Fc domain of IgG molecules is glycosylated at asparagine 297 in the CH2 domain. This asparagine is the site for modification with biantennary-type oligosaccharides. This glycosylation participates in effector functions mediated by Fcγ receptors and complement Clq (Burton and Woof (1992) Adv. Immunol. 51:1-84; Jefferis et al. (1998) Immunol. Rev. 163:59-76). The Fc domain can be produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the antibody of interest, e.g., an antibody described herein. The antibody can be purified from the milk, or for some applications, used directly.

Antibodies can be modified, e.g., with a moiety that improves its stabilization and/or retention in circulation, e.g., in blood, serum, lymph, bronchoalveolar lavage, or other tissues, e.g., by at least 1.5, 2, 5, 10, or 50 fold.

For example, a VLA-4 binding antibody can be associated with a polymer, e.g., a substantially non-antigenic polymer, such as a polyalkylene oxide or a polyethylene oxide.

Suitable polymers will vary substantially by weight. Polymers having molecular number average weights ranging from about 200 to about 35,000 daltons (or about 1,000 to about 15,000, and 2,000 to about 12,500) can be used.

For example, a VLA-4 binding antibody can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g., hyaluronic acid; polymers of sugar alcohols such as polysorbitol and polymannitol; heparin or heparon.

Pharmaceutical Compositions

In some cases, a VLA-4 antagonist, e.g., a VLA-4 binding agent, such as a VLA-4 binding antibody, (e.g., a natalizumab-like antibody molecule (e.g., natalizumab)) can be formulated as a pharmaceutical composition. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible.

A “pharmaceutically acceptable salt” refers to a salt that retains the desired biological activity of the parent compound and does not impart any undesired toxicological effects (see, e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

In some cases, VLA-4 antagonists, e.g., a VLA-4 binding antibody, e.g., a natalizumab-like antibody molecule (e.g., natalizumab), and other agents described herein can be formulated according to standard methods. Exemplary pharmaceutical formulation is described in Gennaro (ed.), Remington: The Science and Practice of Pharmacy, 20.sup.th ed., Lippincott, Williams & Wilkins (2000) (ISBN: 0683306472); Ansel et al., Pharmaceutical Dosage Forms and Drug Delivery Systems, 7.sup.th Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

In one embodiment, a VLA-4 antagonist, e.g., a VLA-4 binding antibody, e.g., a natalizumab-like antibody molecule (e.g., natalizumab) or another agent (e.g., another antibody) can be formulated with excipient materials, such as sodium chloride, sodium dibasic phosphate heptahydrate, sodium monobasic phosphate, and polysorbate 80. It can be provided, for example, in a buffered solution at a concentration of about 20 mg/ml and can be stored at 2-8t. Natalizumab can be formulated as described on the manufacturer's label.

Pharmaceutical compositions may also be in a variety of other forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration.

The composition can be formulated as a solution, microemulsion, dispersion, liposome, or other ordered structure suitable to high drug concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Administration

A VLA-4 antagonist, e.g., a VLA-4 binding antibody, e.g., a natalizumab-like antibody molecule (e.g., natalizumab), can be administered to a subject, e.g., a human subject, by a variety of methods. For many applications, the route of administration is one of: intravenous injection or infusion, subcutaneous injection, or intramuscular injection. A VLA-4 binding antibody, such as a natalizumab-like antibody molecule (e.g., natalizumab), can be administered as a fixed dose, or in a mg/kg dosage. The antibody can be administered intravenously (IV) or subcutaneously (SC). In embodiments, the antibody is administered IV.

In accordance with the methods and compositions described herein, in embodiments, a VLA-4 antagonist, e.g., a natalizumab-like antibody molecule (e.g., natalizumab), is administered to a subject as a single dosage. In embodiments, a VLA-4 antagonist, e.g., a natalizumab-like antibody molecule (e.g., natalizumab), is administered to a subject as multiple dosages. In embodiments, the time at which a first dosage is administered is referred to as t1, the time at which a second dosage is administered is referred to as t2, the time at which a third dosage is administered is referred to as t3, and so on. In embodiments, in a multiple-dosage regimen, the time interval between each dosage is at least 1 day (e.g., at least 1, 2, 3, 4, 5, 6, or 7 days, or 1, 2, 3, 4, 5, or 6 weeks, or 1, 2, 3, 4, 5, or 6 months or more). In embodiments, the time interval between each dosage (e.g., between t1 and t2, and/or between t2 and t3, and so on) is about 2 days (e.g., 36-60 hours, e.g., 40-56 hours, e.g., 44-52 hours, e.g., about 48 hours).

In embodiments, the VLA-4 antagonist, e.g., the VLA-4 binding antibody, e.g., a natalizumab-like antibody molecule (e.g., natalizumab), is administered, e.g., IV, at a dosage of 250 to 350, e.g., about 300, e.g., 300, mg; 350 to 500, e.g., 390 to 450, or 450+/−5%, or about 450, e.g., 450, mg; 550 to 650, e.g., 575 to 625, e.g., 600+/−5%, e.g., about 600, e.g., 600, mg. In embodiments, the VLA-4 antagonist, e.g., the VLA-4 binding antibody, e.g., a natalizumab-like antibody molecule (e.g., natalizumab), is administered at a dosage of 600 mg, e.g., IV, e.g., at a single dosage of 600 mg IV. In embodiments, the VLA-4 antagonist, e.g., the VLA-4 binding antibody, e.g., a natalizumab-like antibody molecule (e.g., natalizumab), is administered at a dosage of 450 mg, e.g., IV, e.g., at a single dosage of 450 mg IV. In embodiments, the VLA-4 antagonist, e.g., the VLA-4 binding antibody, e.g., a natalizumab-like antibody molecule (e.g., natalizumab), is administered at a dosage of 300 mg, e.g., IV, e.g., at a single dosage of 300 mg IV. In embodiments, the VLA-4 antagonist, e.g., the VLA-4 binding antibody, e.g. a natalizumab-like antibody molecule (e.g., natalizumab), is administered, e.g., IV, at a dosage that results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L. In embodiments, the VLA-4 antagonist, e.g., the VLA-4 binding antibody, e.g., a natalizumab-like antibody molecule (e.g., natalizumab), is administered in a specified period from LKN, e.g., within 12 hours or less (e.g., within 12, 11, 10, 9 hours or less), or within 9 hours of less (e.g., within 9, 8, 7, 6, 5, 4, 3, 2, or 1 hour or less) after LKN. In embodiments, the VLA-4 antagonist, e.g., the VLA-4 binding antibody, e.g., a natalizumab-like antibody molecule (e.g., natalizumab), is administered within 6-9 hours (e.g., within 6-7, 7-8, or 8-9 hours) after LKN.

In some embodiments, the VLA-4 antagonist, e.g., the VLA-4 binding antibody molecule, e.g., a natalizumab-like antibody molecule (e.g., natalizumab), is administered, e.g., IV, at a dosage of about 250 to 350, e.g., about 300, e.g., 300, mg; 350 to 500, e.g., 390 to 450, or 450+/−5%, or about 450, e.g., 450, mg; 550 to 650, e.g., 575 to 625, e.g., 600+/−5%, e.g., about 600, e.g., 600, mg within 12 hours or less, e.g., 11, 10, 9, 8, 7, 6, 5, 4, 3, 2 hours or less after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of about 250 to 350, e.g., about 300, e.g., 300, mg; 350 to 500, e.g., 390 to 450, or 450+/−5%, or about 450, e.g., 450, mg; 550 to 650, e.g., 575 to 625, e.g., 600+/−5%, e.g., about 600, e.g., 600, mg within 9 hours or less, e.g., approximately 1 to 9 hours, e.g., 9 to 2 hours, e.g., 9 to 3 hours, e.g., 9 to 4 hours, e.g., 9 to 5 hours, after LKN. In some embodiments, the VLA-4 antagonist, e.g., the VLA-4 binding antibody molecule, e.g., a natalizumab-like antibody molecule (e.g., natalizumab), is administered, e.g., at a dosage of about 250 to 350, e.g., about 300, e.g., 300, mg; 350 to 500, e.g., 390 to 450, or 450+/−5%, or about 450, e.g., 450, mg; 550 to 650, e.g., 575 to 625, e.g., 600+/−5%, e.g., about 600, e.g., 600, mg within 6 hours or less, e.g., 6, 5, 4, 3, 2 or 1 hour after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of 250 to 350, e.g., about 300, e.g., 300, mg; 350 to 500, e.g., 390 to 450, or 450+/−5%, or about 450, e.g., 450, mg; 550 to 650, e.g., 575 to 625, e.g., 600+/−5%, e.g., about 600, e.g., 600, mg within 6 hours after LKN.

In embodiments, where a VLA-4 antagonist, e.g., a natalizumab-like antibody molecule (e.g., natalizumab), is administered to a subject as multiple dosages, each dosage (e.g., at t1, t2, or t3) can be any dosage described herein. In embodiments, the dosage at t1, t2, and/or t3 is 100 to 350 mg, e.g., 150 to 300 mg, e.g., administered IV. For example, the dosage at t1, t2, and/or t3 is 150+/−5%, e.g., about 150 mg, e.g., 150 mg; or 300+/−5%, e.g., about 300 mg, e.g., 300 mg, e.g., administered IV. In embodiments, the dosage at t1 is administered to a subject within 12 hours or less, e.g., 11, 10, 9, 8, 7, 6, 5, 4, 3, 2 hours or less after LKN.

In embodiments, methods herein comprise administering a VLA-4 antagonist, e.g., a natalizumab-like antibody molecule (e.g., natalizumab), subcutaneously. In embodiments, a subcutaneous dosage of the VLA-4 antagonist is calculated by dividing an IV dosage (e.g., an IV dosage described herein) by 0.7. For example, a subcutaneous dosage is calculated by dividing an IV dosage by 0.7, and rounding up or down to the nearest number divisible by 50. For example, an IV dosage of 300 mg corresponds to a subcutaneous dosage of 300 mg divided by 0.7 (429), rounded up or down to the nearest 50; i.e., an IV dosage of 300 mg corresponds to a subcutaneous dosage of 400 mg or 450 mg. For example, an IV dosage of 450 corresponds to a subcutaneous dosage of 450 mg divided by 0.7 (643), rounded up or down to the nearest 50; i.e., an IV dosage of 450 mg corresponds to a subcutaneous dosage of 600 mg or 650 mg. For example, an IV dosage of 600 mg corresponds to a subcutaneous dosage of 600 mg divided by 0.7 (857), rounded up or down to the nearest 50; i.e., an IV dosage of 600 mg corresponds to a subcutaneous dosage of 850 mg or 900 mg. The same applies not only to specific dosages but to ranges of dosages provided herein as well.

In some embodiments, the antibody, e.g., a natalizumab-like antibody molecule (e.g., natalizumab), is administered at a fixed unit dose of between 50-1000 mg IV, e.g., between 100-600 mg IV, e.g., between 200 and 400 mg IV, e.g., about 300 mg IV. In embodiments, when administered subcutaneously, the antibody is administered at a dosage between 50-100 mg SC (e.g., 75 mg). It can also be administered in a bolus at a dosage of between 1 and 10 mg/kg, e.g., about 6.0, 4.0, 3.0, 2.0, 1.0 mg/kg. In some cases, continuous administration may be provided, e.g., via a subcutaneous pump.

In some embodiments, the VLA-4 antagonist, e.g., the VLA-4 binding antibody molecule, is administered to a subject within 12 hours or less, e.g., 11, 10, 9, 8, 7, 6, 5, 4, 3, 2 hours or less after LKN. Preferably, the VLA-4 antagonist, e.g., the VLA-4 binding antibody molecule, e.g., a natalizumab-like antibody molecule (e.g., natalizumab), is administered within 9 hours or less, e.g., approximately 1 to 9 hours, e.g., 9 to 2 hours, e.g., 9 to 3 hours, e.g., 9 to 4 hours, e.g., 9 to 5 hours, after LKN. In preferred embodiments, the VLA-4 antagonist, e.g., the VLA-4 binding antibody molecule, e.g., a natalizumab-like antibody molecule (e.g., natalizumab), is administered within 6 hours or less, e.g., 6, 5, 4, 3, 2 or 1 hour after LKN.

In some embodiments, the VLA-4 antagonist, e.g., the VLA-4 binding antibody molecule, e.g., a natalizumab-like antibody molecule (e.g., natalizumab), is administered at a dosage of between 200 and 400 mg within 12 hours or less, e.g., 11, 10, 9, 8, 7, 6, 5, 4, 3, 2 hours or less after LKN. In some embodiments, the VLA-4 antagonist, e.g., the VLA-4 binding antibody molecule, e.g., a natalizumab-like antibody molecule (e.g., natalizumab), is administered at a dosage of between 200 and 400 mg within 9 hours or less, e.g., approximately 1 to 9 hours, e.g., 9 to 2 hours, e.g., 9 to 3 hours, e.g., 9 to 4 hours, e.g., 9 to 5 hours, after LKN. In some embodiments, the VLA-4 antagonist, e.g., the VLA-4 binding antibody molecule, e.g., a natalizumab-like antibody molecule (e.g., natalizumab), is administered at a dosage of between 200 and 400 mg within 6 hours or less, e.g., 6, 5, 4, 3, 2 or 1 hour after LKN.

In some embodiments, the VLA-4 antagonist, e.g., the VLA-4 binding antibody molecule, e.g., a natalizumab-like antibody molecule (e.g., natalizumab), is administered, e.g., IV, at a dosage of about 300 mg within 12 hours or less, e.g., 11, 10, 9, 8, 7, 6, 5, 4, 3, 2 hours or less after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of about 300 mg within 9 hours or less, e.g., approximately 1 to 9 hours, e.g., 9 to 2 hours, e.g., 9 to 3 hours, e.g., 9 to 4 hours, e.g., 9 to 5 hours, after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of about 300 mg within 6 hours or less, e.g., 6, 5, 4, 3, 2 or 1 hour after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of 300 mg within 6 hours after LKN.

In some embodiments, the VLA-4 antagonist, e.g., the VLA-4 binding antibody molecule, e.g., a natalizumab-like antibody molecule (e.g., natalizumab), is administered at a dosage of between 150 and 450 mg within 12 hours or less, e.g., 11, 10, 9, 8, 7, 6, 5, 4, 3, 2 hours or less after LKN. In some embodiments, the VLA-4 antagonist, e.g., the VLA-4 binding antibody molecule, e.g., a natalizumab-like antibody molecule (e.g., natalizumab), is administered at a dosage of between 150 and 450 mg within 9 hours or less, e.g., approximately 1 to 9 hours, e.g., 9 to 2 hours, e.g., 9 to 3 hours, e.g., 9 to 4 hours, e.g., 9 to 5 hours, after LKN. In some embodiments, the VLA-4 antagonist, e.g., the VLA-4 binding antibody molecule, e.g., a natalizumab-like antibody molecule (e.g., natalizumab), is administered at a dosage of between 150 and 450 mg within 6 hours or less, e.g., 6, 5, 4, 3, 2 or 1 hour after LKN.

In some embodiments, the VLA-4 antagonist, e.g., the VLA-4 binding antibody molecule, e.g., a natalizumab-like antibody molecule (e.g., natalizumab), is administered at a dosage of about 150 mg within 12 hours or less, e.g., 11, 10, 9, 8, 7, 6, 5, 4, 3, 2 hours or less after LKN. In some embodiments, the VLA-4 antagonist, e.g., the VLA-4 binding antibody molecule, e.g., a natalizumab-like antibody molecule (e.g., natalizumab), is administered at a dosage of about 150 mg within 9 hours or less, e.g., approximately 1 to 9 hours, e.g., 9 to 2 hours, e.g., 9 to 3 hours, e.g., 9 to 4 hours, e.g., 9 to 5 hours, after LKN. In some embodiments, the VLA-4 antagonist, e.g., the VLA-4 binding antibody molecule, e.g., a natalizumab-like antibody molecule (e.g., natalizumab), is administered at a dosage of about 150 mg within 6 hours or less, e.g., 6, 5, 4, 3, 2 or 1 hour after LKN. In some embodiments, the VLA-4 antagonist, e.g., the VLA-4 binding antibody molecule, e.g., a natalizumab-like antibody molecule (e.g., natalizumab), is administered at a dosage of 150 mg within 6 hours after LKN.

In some embodiments, the VLA-4 antagonist, e.g., the VLA-4 binding antibody molecule, e.g., a natalizumab-like antibody molecule (e.g., natalizumab), is administered, e.g., IV, at a dosage of about 450 mg within 12 hours or less, e.g., 11, 10, 9, 8, 7, 6, 5, 4, 3, 2 hours or less after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of about 450 mg within 9 hours or less, e.g., approximately 1 to 9 hours, e.g., 9 to 2 hours, e.g., 9 to 3 hours, e.g., 9 to 4 hours, e.g., 9 to 5 hours, after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of about 450 mg within 6 hours or less, e.g., 6, 5, 4, 3, 2 or 1 hour after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of 450 mg within 6 hours after LKN.

In some embodiments, the VLA-4 antagonist, e.g., the VLA-4 binding antibody molecule, e.g., a natalizumab-like antibody molecule (e.g., natalizumab), is administered, e.g., IV, at a dosage of about 600 mg (e.g., 600 mg) within 12 hours or less, e.g., 11, 10, 9, 8, 7, 6, 5, 4, 3, 2 hours or less after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of about 600 mg (e.g., 600 mg) within 9 hours or less, e.g., approximately 1 to 9 hours, e.g., 9 to 2 hours, e.g., 9 to 3 hours, e.g., 9 to 4 hours, e.g., 9 to 5 hours, after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of about 600 mg (e.g., 600 mg) within 6 hours or less, e.g., 6, 5, 4, 3, 2 or 1 hour after LKN. In some embodiments, the VLA-4 antagonist, e.g., the VLA-4 binding antibody molecule, e.g., a natalizumab-like antibody molecule (e.g., natalizumab), is administered, e.g., IV, at a dosage of 600 mg within 6 hours after LKN.

The dosage can also be chosen to reduce or avoid production of antibodies against the VLA-4 binding antibody, to achieve greater than 40, 50, 70, 75, or 80% saturation of the α4 subunit, to achieve to less than 80, 70, 60, 50, or 40% saturation of the α4 subunit, or to prevent an increase the level of circulating white blood cells.

In some aspects, also provided herein is a method of treating a human subject having a disorder described herein, e.g., stroke, e.g., an ischemic stroke (e.g., an acute ischemic stroke), or a hemorrhagic stroke (e.g., an intracerebral hemorrhage or a subarachanoid hemorrhage), or a traumatic brain injury (TBI), comprising:

determining, e.g., by receiving information from a third party on the severity of the stroke (or TBI), the severity of the stroke (or TBI), and responsive to that determination, selecting a dosage of a VLA-4 antagonist, e.g., an anti-alpha4 antibody molecule, e.g., a natalizumab-like antibody molecule (e.g., natalizumab)-like antibody molecule, e.g., a natalizumab-like antibody molecule (e.g., natalizumab), thereby treating the human subject.

In some embodiments, the selected dosage is administered to the subject.

In embodiments, the dosage can be selected based on the severity of stroke. For example, a dosage-A (e.g., higher) dosage of the VLA-4 antagonist is selected (e.g., and administered) if the stoke is determined to be a severe stroke. For example, a dosage-B (e.g., lower) dosage is selected (e.g., and administered) if the stroke is determined to be a less than severe stroke. In some examples, dosage-A is higher than dosage-B, e.g., at least 10, 20 or 30% higher. In some embodiments, the dosage-A is 350 to 500, e.g., 390 to 450, or 450+/−5%, or about 450, e.g., 450, mg; 550 to 650, e.g., 575 to 625, e.g., 600+/−5%, e.g., about 600, e.g., 600, mg; and/or results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L. In embodiments, dosage-B is 250 to 390, e.g., 275 to 325, e.g., 300+/−5%, or about 300 mg; and/or results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L.

Severity of stroke can be determined by methods described herein. Use of such methods to determine severity of stroke are described in greater detail herein.

Without wishing to be bound by theory, it is believed that multiple dosages (e.g., by separate administrations) of a VLA-antagonist (e.g., a natalizumab-like antibody molecule (e.g., natalizumab)) may increase AUC and may thereby lead to higher efficacy (e.g., in a greater number of subjects suffering from a disease described herein). Accordingly, in some embodiments, multiple dosages of a VLA-antagonist, e.g., a natalizumab-like antibody molecule (e.g., natalizumab), are administered to the subject. In embodiments, the subject is administered a first dosage of a VLA-4 antagonist, e.g., a natalizumab-like antibody molecule (e.g., natalizumab), at time 1 (t1); a second dosage of the VLA-4 antagonist (e.g., a natalizumab-like antibody molecule (e.g., natalizumab)) at time 2 (t2); and optionally, a third dosage of the VLA-4 antagonist (e.g., a natalizumab-like antibody molecule (e.g., natalizumab)) at time 3 (t3). Exemplary dosing regimens such as those involving multiple dosages are described herein.

In embodiments, the VLA-4 antagonist, e.g., a natalizumab-like antibody molecule (e.g., natalizumab), is formulated as a controlled release or sustained release formulation. For example, the formulation comprises biodegradable and biocompatible polymers, e.g., polyanhydrides, collagen, ethylene vinyl acetate, polyglycolic acid, polylactic acid, and/or polyorthoesters. Methods of preparing sustained or controlled release formulations are described, e.g., in Sustained and Controlled Release Drug Delivery Systems, J. R. Robinson, ed., Marcel Dekker, Inc., New York, 1978.

In embodiments, a composition described herein is delivered in a microencapsulated delivery system or a medical device, e.g., implant. In embodiments, the composition is delivered by a needleness hypodermic injection device (see, e.g., U.S. Pat. Nos. 5,399,163, 5,383,851, 5,312,335, 5,064,413, 4,941,880, 4,790,824, or 4,596,556). In embodiments, the composition is delivered in an implantable micro-infusion pump that dispenses at a controlled rate (see, e.g., U.S. Pat. No. 4,487,603). In embodiments, the composition is delivered through the skin in a device such as that described in U.S. Pat. No. 4,486,194. In embodiments, the composition is delivered in an infusion pump (see, e.g., U.S. Pat. No. 4,447,233 or 4,447,224). In embodiments, the composition is delivered in an osmotic delivery system with multi-chamber compartments (see, e.g., U.S. Pat. No. 4,475,196).

Methods described herein can also include administering a VLA-4 antagonist in combination with another therapeutic modality, e.g., an additional agent (e.g., a pharmacological agent) or a procedure. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The VLA-4 antagonist and the at least one additional therapeutic agent can be administered simultaneously, in the same or in separate compositions, or sequentially. For sequential administration, the antagonist can be administered first, and the additional agent can be administered second, or the order of administration can be reversed.

The additional agent is preferably an agent with some degree of therapeutic efficacy in treating acute brain injury. Such agents may include, but are not limited to, thrombolytic agents such as plasminogen, tissue plasminogen activator (t-PA) or urokinase, agents that target excitotoxic mechanisms such as Selfotel™ or Aptiganel™, agents that target nitric oxide associated neuronal damage such as Lubeluzole™, agents that target ischemia associated neuronal cellular membrane damage such as Tirilizad™, agents that target anti-inflammatory mechanisms such as Enlimomab™. The agent may be combined with the VLA-4 antagonists either prior to, during, or after administration of the antagonists.

### Methods of Treatment

In some aspects, provided herein are methods of treatment comprising administering an effective amount of a VLA-4 antagonist, e.g., a natalizumab-like antibody molecule (e.g., natalizumab), to a subject suffering from a stroke, e.g., ischemic stroke (e.g., acute ischemic stroke), or a hemorrhagic stroke (e.g., intracerebral hemorrhage or a subarachnoid hemorrhage), ora TBI.

In embodiments, the treatment ameliorates one or more symptoms of the stroke, subarachnoid hemorrhage, or TBI, improves a functional outcome (e.g., an endpoint described herein) of the stroke, subarachnoid hemorrhage, or TBI, and/or prevents further deterioration from the disease.

In embodiments, the VLA-4 antagonist, e.g., a natalizumab-like antibody molecule (e.g., natalizumab), is administered in a specified period from LKN of the stroke, subarachnoid hemorrhage, or TBI, e.g., within 9 hours of less (e.g., within 9, 8, 7, 6, 5, 4, 3, 2, or 1 hour or less) after LKN of the stroke, subarachnoid hemorrhage, or TBI. In embodiments, the VLA-4 antagonist, e.g., a natalizumab-like antibody molecule (e.g., natalizumab), is administered within 6-9 hours (e.g., within 6-7, 7-8, or 8-9 hours) after LKN of the stroke, subarachnoid hemorrhage, or TBI.

In embodiments, onset time of stroke, subarachnoid hemorrhage, or TBI is determined by a medical professional, e.g., by questioning regarding one or more symptom(s) of stroke, subarachnoid hemorrhage, or TBI, e.g., start of symptom(s) of stroke, subarachnoid hemorrhage, or TBI, or time of subject awakening with a stroke, subarachnoid hemorrhage, or TBI. In embodiments, brain imaging, e.g., MRI, is used to determine the time of onset and/or duration of stroke, subarachnoid hemorrhage, or TBI in a subject. See, e.g., Petkova et al. Radiology 257.3(2010):782-92. In some embodiments, e.g., in cases where a stroke, subarachnoid hemorrhage, or TBI onset time is not easily determined, the stroke, subarachnoid hemorrhage, or TBI onset time is determined by the last time the subject was known, e.g., as identified by the subject or another individual, to be well (e.g., last known normal (LKN)). In embodiments, the onset of a TBI is the time of trauma/impact to the head or body, e.g., determined by the subject or another individual (e.g., observer).

In embodiments, a subject is administered a therapy described herein, e.g., a natalizumab-like antibody molecule (e.g., natalizumab), within 6 hours (e.g., within 0, 1, 2, 3, 4, 5, or 6 hours) of the last known normal (LKN). In embodiments, a subject is administered a therapy described herein, e.g., a natalizumab-like antibody molecule (e.g., natalizumab), >6 hours and ≤9 hours of the LKN (e.g., >6, 6.5, 7, 7.5, 8, or 8.5 hours and ≤9, 8.5, 8, 7.5, 7, 6.5 hours of the LKN). In embodiments, a subject is administered a therapy described herein, e.g., a natalizumab-like antibody molecule (e.g., natalizumab), within 12 hours (e.g., within 12, 11, 10, 9 hours or less) of the last known normal (LKN).

In embodiments, the subject suffers from a stroke, e.g., ischemic stroke (e.g., acute ischemic stroke), or a hemorrhagic stroke (e.g., intracerebral hemorrhage or subarachnoid hemorrhage). In embodiments, the subject is a first-time stroke (e.g., ischemic stroke, e.g., acute ischemic stroke; or hemorrhagic stroke (e.g., intracerebral hemorrhage or subarachnoid hemorrhage) sufferer. In embodiments, the subject has previously had a stroke (e.g., ischemic stroke, e.g., acute ischemic stroke; or hemorrhagic stroke (e.g., intracerebral hemorrhage or subarachnoid hemorrhage)).

In embodiments, the subject suffers from an acute middle cerebral artery (MCA) ischemic stroke.

In an ischemic stroke, thrombosis, (e.g., venous thrombosis), embolism, or systemic hypoperfusion causes a disruption of the blood supply to the brain, causing ischemia (lack of oxygen and glucose supply), and as a result, the subject develops a loss of brain function(s). Loss of brain function(s) in the affected area causes functional disabilities, e.g., inability to move one or more limbs on one side of the body, inability to formulate or understand speech, and/or inability to see one side of the visual field. In some cases, a stroke can cause permanent neurological damage, complications, and/or death.

Exemplary symptoms of ischemic stroke include but are not limited to numbness; decreased feeling and/or muscle weakness in the face; dropping of eyelid(s) (ptosis); weakened ocular muscles; hemiplegia; reduced vibratory or sensory sensation; changes in taste, hearing, smell or vision (partial or complete); tongue weakness (inability to move side-to-side or protrude); decreased reflexes; problems with balance; nystagmus; changes in heart rate and breathing; weakened sternocleidomastoid music with inability to turn head to one side; visual field defect(s); aphasia; hemineglect; apraxia; memory deficits; trouble walking; changes in movement coordination; vertigo; disequilibrium; confusion; hypersexual gestures; disorganized thinking; and/or anosognosia.

In hemorrhagic stroke (e.g., intracerebral hemorrhage or subarachnoid hemorrhage), blood vessels in the brain leak or rupture, resulting in bleeding in or around the brain. Damage can occur rapidly due to the pressure of growing amounts of blood and/or because of the blood itself, which irritates the brain tissue, causing it to swell. Symptoms include loss of consciousness, headache, and vomiting. A type of hemorrhagic stroke includes intracerebral hemorrhage.

In intracerebral hemorrhage, a blood vessel inside the brain bursts and leaks blood into surrounding brain tissue. The bleeding causes death of brain cells and loss of normal function in the affected part of the brain. The most common causes of this type of stroke are high blood pressure and aging blood vessels. In some cases, intracerebral hemorrhagic stroke can be caused by an arteriovenous malformation (AVM), which is a genetic condition causing an abnormal connection between veins and arteries that most commonly occurs in the brain or spine. AVM that occurs in the brain can cause vessel breakage and bleeding into the brain.

In embodiments, the subject suffers from subarachnoid hemorrhage (SAH). Subarachnoid hemorrhage (SAH) is characterized by bleeding around the brain and is caused by bleeding into the subarachnoid space (the area between the brain and the tissue covering the brain). Bleeding can happen spontaneously, e.g., from a ruptured cerebral aneurysm, or may result from a head injury, AVM, bleeding disorders or blood thinners. Exemplary symptoms of SAH include a severe headache with a rapid onset, vomiting, confusion or a lowered level of consciousness, and seizures in some cases. Diagnosis of SAH can be made by a CT scan or lumbar puncture. Current treatment methods include neurosurgery or radiologically guided interventions with medications to prevent recurrence of the bleeding and other complications.

In embodiments, the subject suffers from a traumatic brain injury (TBI). Traumatic brain injury occurs when an external mechanical force (e.g., by a violent blow or jolt to the body or head, or an object that penetrates the skull) results in brain dysfunction. Mild TBI can cause temporary dysfunction of brain cells. More Serious™ can result in torn tissues, bruising, bleeding and other physical damage to the brain that can lead to long-term complications or death. For example, a TBI includes a concussion or post-concussion syndrome.

Symptoms of mild TBI include loss of consciousness for a few seconds to a few minutes; a state of being dazed, confused or disoriented; headache; nausea or vomiting; fatigue or drowsiness; difficulty sleeping; sleeping more than usual; dizziness or loss of balance; blurred vision; ringing in the ears; a bad taste in the mouth; changes in the ability to smell; sensitivity to light or sound; memory or concentration problems; mood changes or mood swings; feeling depressed or anxious. Symptoms of moderate to severe TBI include loss of consciousness from several minutes to hours persistent headache or headache that worsens; repeated vomiting or nausea; convulsions or seizures; dilation of one or both pupils of the eyes; clear fluids draining from the nose or ears; inability to awaken from sleep; weakness or numbness in fingers and toes; loss of coordination; profound confusion; agitation, combativeness or other unusual behavior; slurred speech; and coma and other disorders of consciousness.

A test/diagnosis of TBI is the Glasgow Coma Scale, which is a 15-point test that permits the assessment of the initial severity of a brain injury by checking a subject's ability to follow directions and move their eyes and limbs. Higher scores indicate less severe injuries. Other tests/diagnoses of TBI are imaging tests, for example, computerized tomography (CT) scans or magnetic resonance imaging (MRI). An intracranial pressure monitor may also be used to determine amount of tissue swelling from a TBI, as tissue swelling can increase pressure inside the skull and cause additional damage to the brain.

Current treatments/procedures for TBI include diuretics, anti-seizure drugs, coma-inducing drugs, surgery, removal of clotted blood (hematomas), repair of skull fractures, opening of a window in the skull, and/or rehabilitation.

In embodiments, the methods comprise treating the subject with a therapy described herein, e.g., VLA-4 antagonist, e.g., a natalizumab-like antibody molecule (e.g., natalizumab), in combination with a second therapy or procedure, e.g., thrombolysis (e.g., tissue plasminogen activator (tPA), thrombectomy, angioplasty, stenting, therapeutic hypothermia, and/or a medication (e.g., aspirin, clopidrogrel, and/or dipyridamole). In embodiments, the second therapy comprises a thrombolytic agent, anti-inflammatory agent, cytokine, growth factor, steroid, or neuroprotective agent. Exemplary thrombolytic agents include tPA and urokinase. Exemplary neuroprotective agents include an agonist to a receptor, such as N-Methyl-D aspartate (NMDA) receptor, glycine receptor, calcium channel receptor, α-amino-3-hydroxy-5-methyl-4-isoxazoleproprionic acid (AMPA) receptor, sodium channel receptor, bradykinin B2 receptor, bradykinin B1 receptor, adenosine A1 receptor, or α-amino butyric acid (GABA) receptor. Exemplary anti-inflammatory agents include tumor necrosis family members and interleukin-1 (IL-1).

In embodiments, the second therapy or procedure is administered or performed concurrently with (e.g., within 2 days or day of) administration of the therapy described herein, e.g., a natalizumab-like antibody molecule (e.g., natalizumab). In embodiments, the second therapy or procedure is administered or performed prior to or subsequent to administration of the therapy described herein, e.g., a natalizumab-like antibody molecule (e.g., natalizumab).

The disclosure provides methods of treating (e.g., stabilizing, reducing, or eliminating one or more symptoms or stabilizing the subject's score on a stroke scale) stroke, e.g., acute ischemic stroke, by administering a VLA-4 antagonist to a subject having or suspected of having a stroke). The disclosure also provides methods of preventing stroke, e.g., ischemic stroke (e.g., acute ischemic stroke), or a hemorrhagic stroke (e.g., intracerebral hemorrhage), or a subarachnoid hemorrhage, or a symptom thereof by administering a VLA-4 antagonist to a subject at risk of developing a stroke (e.g., a subject that has experienced systemic hypoperfusion) or a subarachnoid hemorrhage.

Patient Subgroup—Higher Stroke Severity at Baseline

In accordance with the methods and compositions described herein, in an aspect, the subject has a severe stroke, e.g., a high NIHSS score (e.g., at least 15, e.g., at least 21, e.g., 21-42) at baseline, e.g., prior to treatment with a therapy described herein, e.g., a natalizumab-like antibody molecule (e.g., natalizumab).

In an aspect, the methods described herein comprise identifying a subject having a severe stroke at baseline, and administering a VLA-4 antagonist, e.g., a natalizumab-like antibody molecule (e.g., natalizumab), to the subject. In embodiments, a subject identified as having a severe stroke has a NIHSS score at baseline of at least 15, e.g., at least 21, e.g., 21-42, e.g., 21-25, 25-30, or 30-42.

In embodiments, the subject having (e.g., identified as having) a severe stroke at baseline is administered a VLA-4 antagonist, e.g., a natalizumab-like antibody molecule (e.g., natalizumab), at a dosage described herein, e.g., via a route of administration described herein, e.g., at 12 hours or less (e.g., 12, 11, 10, 9, 8, 7, 6, 5, 4 3, 2, 1, or less, or within 9 hours, or within 6-9 hours) after LKN.

Patient Subgroup—Lower Stroke Severity (Less than Severe Stroke) at Baseline

In accordance with the methods and compositions described herein, in an aspect, the subject has a less than severe stroke, e.g., a NIHSS score of less than 15 at baseline, e.g., prior to treatment with a therapy described herein, e.g., a natalizumab-like antibody molecule (e.g., natalizumab).

In an aspect, the methods described herein comprise identifying a subject having a less than severe stroke at baseline, and administering a VLA-4 antagonist, e.g., a natalizumab-like antibody molecule (e.g., natalizumab), to the subject. In embodiments, a subject identified as having a less than severe stroke has a NIHSS score at baseline of less than 15.

In embodiments, the subject having (e.g., identified as having) a less than severe stroke at baseline is administered a VLA-4 antagonist, e.g., a natalizumab-like antibody molecule (e.g., natalizumab), at a dosage described herein, e.g., via a route of administration described herein, e.g., at 12 hours or less (e.g., 12, 11, 10, 9, 8, 7, 6, 5, 4 3, 2, 1, or less, or within 9 hours, or within 6-9 hours) after LKN.

Patient Subgroup—Small to Medium Infarct Size at Baseline

In accordance with the methods and compositions described herein, in an aspect, the subject has a small to medium sized infarct at baseline, e.g., prior to treatment with a therapy described herein, e.g., a natalizumab-like antibody molecule (e.g., natalizumab). In embodiments, the subject does not have a large sized infarct at baseline, e.g., prior to treatment with a therapy described herein, e.g., a natalizumab-like antibody molecule (e.g., natalizumab).

In embodiments, the subject has a lesion size of <4.6 cm in diameter (e.g., less than 4.6, 4.5, 4.4, 4.3, 4.2, 4.1, 4, 3.75, 3.5, 3.25, 3, 2.75, 2.5, 2.25, 2, 1.75, 1.5, 1.25, 1, 0.75, 0.5 cm, or less in diameter) at baseline, e.g., prior to treatment with a therapy described herein, e.g., a natalizumab-like antibody molecule (e.g., natalizumab). In embodiments, the subject has a lesion size of no greater than 4.6 cm in diameter (e.g., no greater than 4.6, 4.5, 4.4, 4.3, 4.2, 4.1, 4, 3.75, 3.5, 3.25, 3, 2.75, 2.5, 2.25, 2, 1.75, 1.5, 1.25, 1, 0.75, 0.5 cm, or less in diameter) at baseline, e.g., prior to treatment with a therapy described herein, e.g., a natalizumab-like antibody molecule (e.g., natalizumab). In embodiments, the subject has a lesion size of 2-4 cm in diameter at baseline, e.g., prior to treatment with a therapy described herein, e.g., a natalizumab-like antibody molecule (e.g., natalizumab).

In embodiments, the subject has an infarct volume of less than one-third of the middle cerebral artery (MCA) territory, e.g., the region of brain tissue supplied by the MCA. In embodiments, the subject has an infarct volume of no greater than one-third of the MCA territory.

In an aspect, the methods described herein comprise identifying a subject having a small to medium sized infarct at baseline, and administering a VLA-4 antagonist, e.g., a natalizumab-like antibody molecule (e.g., natalizumab), to the subject.

In embodiments, a subject identified as having a small to medium sized infarct has a lesion size of <4.6 cm in diameter (e.g., less than 4.6, 4.5, 4.4, 4.3, 4.2, 4.1, 4, 3.75, 3.5, 3.25, 3, 2.75, 2.5, 2.25, 2, 1.75, 1.5, 1.25, 1, 0.75, 0.5 cm, or less in diameter) at baseline, e.g., prior to treatment with a therapy described herein, e.g., a natalizumab-like antibody molecule (e.g., natalizumab). In embodiments, a subject identified as having a small to medium sized infarct subject has a lesion size of no greater than 4.6 cm in diameter (e.g., no greater than 4.6, 4.5, 4.4, 4.3, 4.2, 4.1, 4, 3.75, 3.5, 3.25, 3, 2.75, 2.5, 2.25, 2, 1.75, 1.5, 1.25, 1, 0.75, 0.5 cm, or less in diameter) at baseline, e.g., prior to treatment with a therapy described herein, e.g., a natalizumab-like antibody molecule (e.g., natalizumab). In embodiments, a subject identified as having a small to medium sized infarct subject has a lesion size of 2-4 cm in diameter at baseline, e.g., prior to treatment with a therapy described herein, e.g., a natalizumab-like antibody molecule (e.g., natalizumab).

In embodiments, a subject identified as having a small to medium sized infarct subject has an infarct volume of less than one-third of the middle cerebral artery (MCA) territory, e.g., the region of brain tissue supplied by the MCA. In embodiments, a subject identified as having a small to medium sized infarct subject has an infarct volume of no greater than one-third of the MCA territory.

In embodiments, the subject having (e.g., identified as having) a small to medium sized infarct at baseline is administered a VLA-4 antagonist, e.g., a natalizumab-like antibody molecule (e.g., natalizumab), at a dosage described herein, e.g., via a route of administration described herein, e.g., at 12 hours or less (e.g., 12, 11, 10, 9, 8, 7, 6, 5, 4 3, 2, 1, or less, or within 9 hours, or within 6-9 hours) after LKN.

Patient Subgroup—Large Infarct Size at Baseline

In accordance with the methods and compositions described herein, in an aspect, the subject has a large sized infarct at baseline, e.g., prior to treatment with a therapy described herein, e.g., a natalizumab-like antibody molecule (e.g., natalizumab). In embodiments, the subject does not have a small to medium sized infarct at baseline, e.g., prior to treatment with a therapy described herein, e.g., a natalizumab-like antibody molecule (e.g., natalizumab).

In embodiments, the subject has a lesion size of at least 4.6 cm in diameter (e.g., at least 4.6, 4.8, 5, 5.2, 5.4, 5.6 5.8, 6, 7, 8, 9, 10 cm, or more in diameter) at baseline, e.g., prior to treatment with a therapy described herein, e.g., a natalizumab-like antibody molecule (e.g., natalizumab). In embodiments, the subject has an infarct volume of at least one-third (e.g. at least one-third, two-thirds, or three-fourths, or more) of the middle cerebral artery (MCA) territory, e.g., the region of brain tissue supplied by the MCA.

In an aspect, the methods described herein comprise identifying a subject having a large sized infarct at baseline, and administering a VLA-4 antagonist, e.g., a natalizumab-like antibody molecule (e.g., natalizumab), to the subject.

In embodiments, a subject identified as having a large sized infarct has a lesion size of at least 4.6 cm in diameter (e.g., at least 4.6, 4.8, 5, 5.2, 5.4, 5.6 5.8, 6, 7, 8, 9, 10 cm, or more in diameter) at baseline, e.g., prior to treatment with a therapy described herein, e.g., a natalizumab-like antibody molecule (e.g., natalizumab). In embodiments, a subject identified as having a small to medium sized infarct subject has an infarct volume of at least one-third (e.g. at least one-third, two-thirds, or three-fourths, or more) of the middle cerebral artery (MCA) territory, e.g., the region of brain tissue supplied by the MCA.

In embodiments, the subject having (e.g., identified as having) a large sized infarct at baseline is administered a VLA-4 antagonist, e.g., a natalizumab-like antibody molecule (e.g., natalizumab), at a dosage described herein, e.g., via a route of administration described herein, e.g., at 12 hours or less (e.g., 12, 11, 10, 9, 8, 7, 6, 5, 4 3, 2, 1, or less, or within 9 hours, or within 6-9 hours) after LKN.

Patient Subgroup—with Tissue Plasminogen Activator (tPA) Use or without

In accordance with the methods and compositions described herein, in embodiments, the subject has been treated with a tissue plasminogen activator (tPA) prior to administration with a therapy described herein, e.g., a natalizumab-like antibody molecule (e.g., natalizumab). In embodiments, the subject has been treated with a tissue plasminogen activator (tPA) at least 1 day prior (e.g., at least 1, 2, 3, 4, 5, 6, 7 days, 1, 2, 3, 4, 5, 6, 7, 8 weeks, 1, 2, 3, 4, 5, 6, 7, 8, 9 10, 11, or 12 months prior) to administration with a therapy described herein, e.g., a natalizumab-like antibody molecule (e.g., natalizumab). In embodiments, the subject has been treated with a tPA within 12 months (e.g., within 12, 11, 10, 9, 8, 7, 6, 5, 4, 3, 2, or 1 month, within 8, 7, 6, 5, 4, 3, 2, or 1 week, within 7, 6, 5, 4, 3, 2, or 1 day) of administration with a therapy described herein, e.g., a natalizumab-like antibody molecule (e.g., natalizumab).

In embodiments, the subject has not been treated with a tPA prior to administration with a therapy described herein, e.g., a natalizumab-like antibody molecule (e.g., natalizumab).

In embodiments, the subject is administered a tPA in combination with a therapy described herein, e.g., a natalizumab-like antibody molecule (e.g., natalizumab). In embodiments, the subject is administered a tPA concurrently with a therapy described herein, e.g., a natalizumab-like antibody molecule (e.g., natalizumab). In embodiments, the subject is administered a tPA prior to (e.g., within 4 weeks, 3 weeks, 2 weeks, 1 week, 7 days, 6 days, 5 days, 4 days, 3 days, 2 days, 1 day, 24 hours, 12 hours, 6 hours, 5 hours, 4 hours, 3 hours, 2 hours, 1 hour, or less, prior to) a therapy described herein, e.g., a natalizumab-like antibody molecule (e.g., natalizumab). In embodiments, the subject is administered a tPA subsequent to (e.g., within 4 weeks, 3 weeks, 2 weeks, 1 week, 7 days, 6 days, 5 days, 4 days, 3 days, 2 days, 1 day, 24 hours, 12 hours, 6 hours, 5 hours, 4 hours, 3 hours, 2 hours, 1 hour, or less, subsequent to) a therapy described herein, e.g., a natalizumab-like antibody molecule (e.g., natalizumab).

In embodiments, the subject is not administered a tPA is combination in with a therapy described herein, e.g., a natalizumab-like antibody molecule (e.g., natalizumab).

### Endpoints

Standard tests for neurological recovery (e.g., National Institute of Health Stroke Scale (NIHSS), Barthel Index, modified Rankin Scale (mRS), Glasgow Outcome Scale, Montreal Cognative Assessment (MoCA), Stroke Impact Scale (SIS-16)) can be employed by skilled artisans to determine efficacy or rehabilitation. The NIHSS classifies the severity of a stroke based on a subject's ability to answer questions and perform activities relating to level of consciousness, language, visual-field loss, extraocular movement, motor strength, ataxia, dysarthria, sensory loss and extinction and inattention. There are 15 items and ratings for each item are scored with 3 to 5 grades with 0 as normal and a maximum severity score of 42 for all items. A NIHSS of 1-5 is indicative of a mild stroke; a score of 5-14 is indicative of a mild to moderate stroke, a score of 15-24 is indicative of a severe stroke; and a score of greater than 25 is indicative of a very severe stroke. See, e.g., Brott et al. Stroke. 20(1989):864-70.

In accordance with the compositions and methods described herein, a patient is evaluated by one or more endpoints to assess the efficacy of a treatment described herein and/or the safety/tolerability of a treatment described herein. Exemplary endpoints include endpoints assessed by imaging techniques, such as magnetic resonance imaging (MRI), clinical efficacy endpoints, or safety/tolerability endpoints. Exemplary endpoints include but are not limited to infarct volume (e.g., change in infarct volume from baseline, e.g., detected by imaging); clinical endpoints, such as modified Rankin Scale (mRS), NIHSS, Barthel index, stroke-impact scale-16, Montreal cognitive assessment; and safety/tolerability endpoints. Stroke outcomes/endpoints measure different but related aspects of disability. For example, the mRS measures functional independence, the NIHSS measures key components of standard neurological exam, and the Barthel index assesses activity related to self care and mobility. See, e.g., Kasner Lancet Neurology 5.7(2006):603-12.

Imaging (e.g., by MRI)

In embodiments, the infarct volume is measured in a patient after stroke, e.g., before and/or after treatment with a therapy described herein, e.g., a natalizumab-like antibody molecule (e.g., natalizumab). In embodiments, imaging (e.g., by MRI) is used to measure the infarct volume in a patient after stroke. Infarct volume refers to the lesion size in a brain of a subject who has suffered a stroke, e.g., ischemic stroke. Lesions occur after ischemic stroke because the lack of oxygen delivered to tissues at the site of the lesion cause tissue damage and necrosis. Infarct volume can be useful in determining the stroke sub-type and predicting the clinical condition and outcome of a stroke subject. Measurements of infarct volume can also be used to assess the efficacy of stroke therapies. Infarct volume can be measured using imaging techniques, such as CT scans and MRI.

In embodiments, infarct volume is measured using diffusion weighted imaging (DWI), which is a form of MRI. See, e.g., van Everdingen et al. Stroke. 29(1998):1783-90; or Barrett et al. Stroke. 40(2009):2422-27, incorporated herein by reference. In embodiments, DWI permits early identification (e.g., within several hours) of ischemic stroke, e.g., acute ischemic stroke.

In embodiments, imaging (e.g., by MRI) is used to detect a change in infarct volume growth from baseline after administration of a therapy described herein, e.g., a natalizumab-like antibody molecule (e.g., natalizumab). In embodiments, the change in infarct volume from baseline (e.g., before administration of therapy) is measured 6 hours or later (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more) after a first administration of a therapy described herein, e.g., a natalizumab-like antibody molecule (e.g., natalizumab).

mRS Scale (a Clinical Endpoint)

In embodiments, a patient is evaluated for improvement in functional independence after treatment (e.g., with a treatment described herein, e.g., a natalizumab-like antibody molecule (e.g., natalizumab)). In embodiments, the patient is evaluated by the modified Rankin Scale (mRS), which is a measure of functional independence. Table 1 describes the scoring method on the mRS scale. A lower score indicates greater functional independence. A higher score indicates less functional independence/greater disability.

TABLE 1
Modified Rankin Scale (mRS)

| Score | Description |
|---|---|
| 0 | No symptoms at all |
| 1 | No significant disability despite symptoms; able to carry |
| | out all duties and activities |
| 2 | Slight disability; unable to carry out all previous activities, |
| | but able to look after own affairs without assistance |
| 3 | Moderate disability; required some help but able to walk |
| | without assistance |
| 4 | Moderately severe disability; unable to walk without assistance |
| | and unable to attend to own bodily needs without assistance |
| 5 | Severe disability; bedridden, inconsistent and requiring |
| | constant nursing care and attention |
| 6 | dead |

In embodiments, the mRS score is determined in a patient 6 hours or later (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more) after a first administration of a therapy described herein, e.g., a natalizumab-like antibody molecule (e.g., natalizumab). In embodiments, a patient has a lower mRS score after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) compared to before treatment. In embodiments, a patient has a mRS score of 3 or less after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy). In embodiments, a patient has a mRS score of 0 or 1 after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy).

In embodiments, an excellent outcome is defined as an mRS of 0 or 1 (0,1). In embodiments, an excellent outcome on the mRS scale indicates that the therapy is efficacious, e.g., that the patient improved in functional independence after treatment (e.g., with a treatment described herein, e.g., a natalizumab-like antibody molecule (e.g., natalizumab)).

In embodiments, a lower mRS score after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) compared to before treatment indicates improvement, e.g., that the patient improved in functional independence after treatment (e.g., with a treatment described herein, e.g., a natalizumab-like antibody molecule (e.g., natalizumab)).

In embodiments, a mRS score of 3 or less after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) indicates improvement, e.g., that the patient improved in functional independence after treatment (e.g., with a treatment described herein, e.g., a natalizumab-like antibody molecule (e.g., natalizumab)).

Barthel Index (BI) (a Clinical Endpoint)

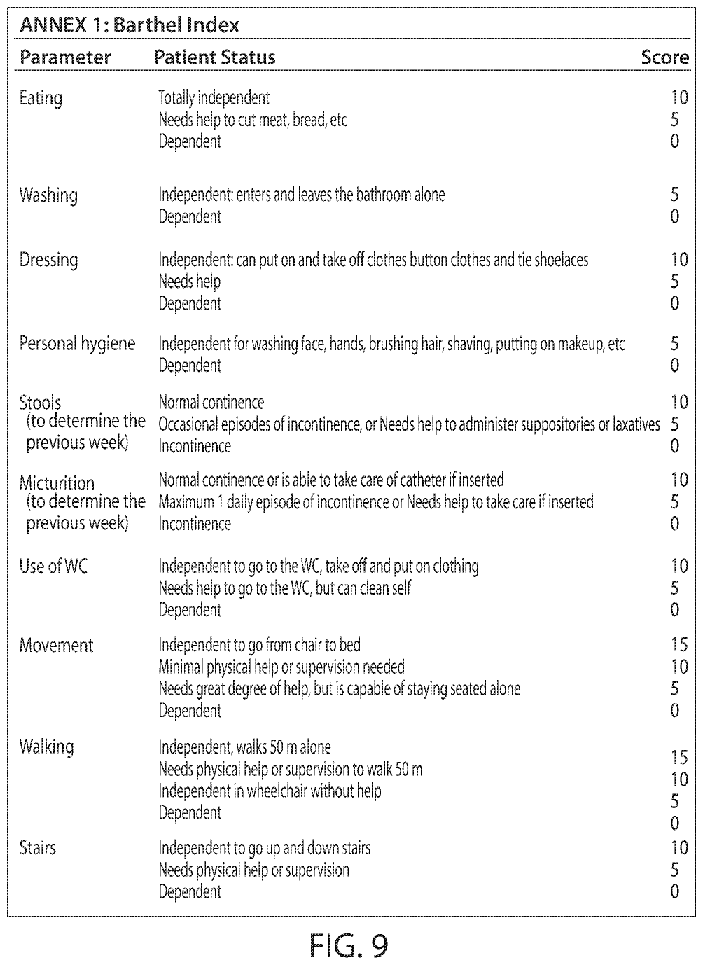

The BI is a measure of activities of daily living and how independent a patient is in performing various activities. A description of the scoring method for BI is shown in FIG. 9. A patient's score for each activity is summed. Higher scores indicate greater independence; lower scores indicate greater disability.

In embodiments, the BI score is determined in a patient 6 hours or later (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more) after a first administration of a therapy described herein, e.g., a natalizumab-like antibody molecule (e.g., natalizumab). In embodiments, a patient has a higher BI score after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) compared to before treatment. In embodiments, a patient has a BI score of 85 or more after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy). In embodiments, a patient has a BI score of at least 95 after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy).

In embodiments, an excellent outcome is defined as a BI score of at least 95. In embodiments, an excellent outcome on the BI scale indicates that the therapy is efficacious, e.g., that the patient improved in functional independence in daily activities after treatment (e.g., with a treatment described herein, e.g., a natalizumab-like antibody molecule (e.g., natalizumab)).

In embodiments, a functional outcome is defined as a BI score of at least 85. In embodiments, a functional outcome on the BI scale indicates that the therapy is efficacious, e.g., that the patient improved in functional independence in daily activities after treatment (e.g., with a treatment described herein, e.g., a natalizumab-like antibody molecule (e.g., natalizumab)).

In embodiments, a higher BI score after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) compared to before treatment indicates that improvement, e.g., that the patient improved in functional independence in daily activities after treatment (e.g., with a treatment described herein, e.g., a natalizumab-like antibody molecule (e.g., natalizumab)).

National Institutes of Health (NIH) Stroke Scale (NIHSS) (as a Clinical Endpoint)

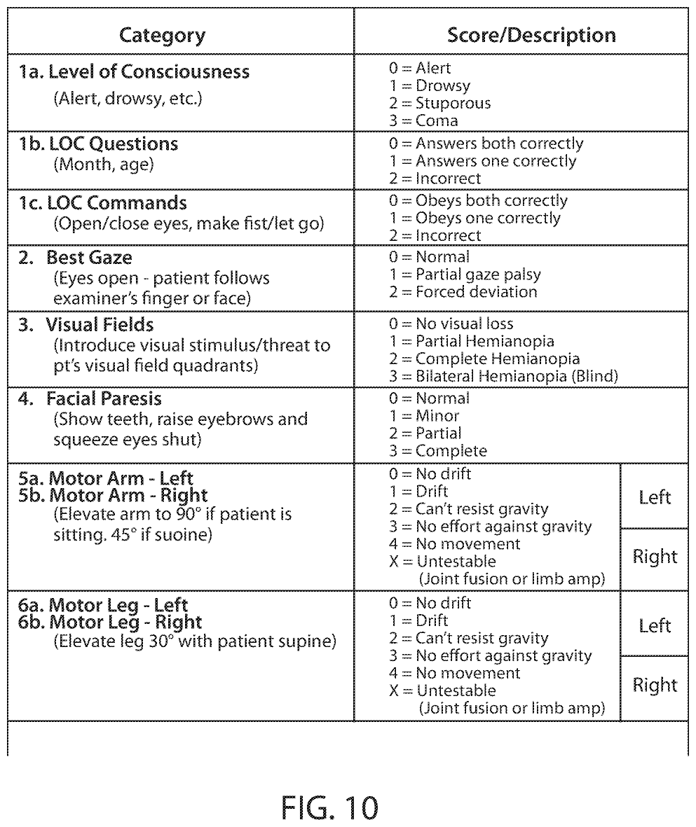

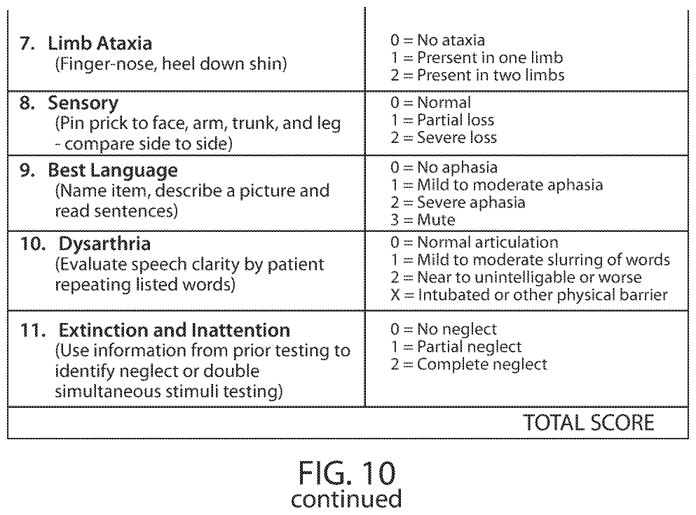

In addition to assessing severity of stroke, the NIHSS can also be a measure of a patient's neurologic function after stroke. NIHSS is an 11 item assessment scored from 0 to 42. A description of the scoring method for NIHSS is shown in FIG. 10. The score for each item is summed to get a total score.

In embodiments, the NIHSS score is determined in a patient 6 hours or later (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more) after a first administration of a therapy described herein, e.g., a natalizumab-like antibody molecule (e.g., natalizumab). In embodiments, a patient has a lower NIHSS score after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) compared to before treatment. In embodiments, a patient has a NIHSS score after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) that is at least 8 points less than before treatment. In embodiments, a patient has a NIHSS score of 0 or 1 after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy). In embodiments, the NIHSS score of a patient does not change after administration of the therapy compared to at baseline.

In embodiments, an excellent outcome is defined as a NIHSS score of 0 or 1 (0,1) or at least 8 points lower than baseline score. In embodiments, an excellent outcome on the NIHSS scale indicates that the therapy is efficacious, e.g., that the patient improved in neurological function after treatment (e.g., with a treatment described herein, e.g., a natalizumab-like antibody molecule (e.g., natalizumab)).

In embodiments, a lower NIHSS score after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) compared to before treatment indicates improvement, e.g., that the patient improved in neurological function after treatment (e.g., with a treatment described herein, e.g., a natalizumab-like antibody molecule (e.g., natalizumab)).

In embodiments, a NIHSS score decrease of at least 8 points after treatment compared to baseline (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) indicates that the therapy is efficacious, e.g., that the patient improved in neurological function after treatment (e.g., with a treatment described herein, e.g., a natalizumab-like antibody molecule (e.g., natalizumab)).

Stroke Impact Scale-16 (SIS-16) (a Clinical Endpoint)

The SIS-16 is a self-report scale assessing the activities of daily living (ADLs) and physical domains after stroke, such as hand function, mobility, communication, emotion, memory and thinking, and participation. See, e.g., Duncan et al. Neurology. 60.2(2003):291-6. The SIS-16 includes 16 items, e.g., 7 ADL items, 8 mobility items, and 1 hand function item. For example, the SIS-16 includes items that cover body function (e.g., bladder and bowel control), activity (e.g., bathing self), and participation (e.g., shopping), with total scores ranging from 0 to 100.

In embodiments, the SIS-16 score is determined in a patient 6 hours or later (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more) after a first administration of a therapy described herein, e.g., a natalizumab-like antibody molecule (e.g., natalizumab). In embodiments, a patient has a higher SIS-16 score after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) compared to before treatment.

In some embodiments, a favorable SIS-16 outcome after administration of a therapy described herein, e.g., a VLA-4 antagonist, e.g., a natalizumab-like antibody molecule (e.g., natalizumab), is a score greater than the median score. In embodiments, a favorable outcome on the SIS-16 scale indicates that the therapy is efficacious, e.g., that the patient improved in self-perceived ADLs and physical capabilities after treatment (e.g., with a treatment described herein, e.g., a natalizumab-like antibody molecule (e.g., natalizumab)).

In embodiments, a higher SIS-16 score after administration of a therapy described herein, e.g., a VLA-4 antagonist, e.g., a natalizumab-like antibody molecule (e.g., natalizumab), compared to at baseline, indicates improvement, e.g., that the patient improved in self-perceived ADLs and physical capabilities after treatment.

MoCA (a clinical endpoint) The Montreal Cognitive Assessment (MoCA) is a cognitive evaluation method. The MoCA is a 30-point test administrable in about 10 minutes that evaluates cognitive domains, such as attention and concentration, language, visuoconstructional skills, visuospatial abilities, conceptual thinking, calculations, executive functions, memory (e.g., short term memory, working memory), recall, and orientation (e.g., to time and space). The MoCA is useful for evaluating a subject who has suffered from a stroke or a subject who is experiencing memory difficulties. See, e.g., Nasreddine et al. The Montreal Cognitive Assessment (MoCA): A brief cognitive screening tool for detection of mild cognitive impairment. Neurology, 62(7): S5, A132. Presented at the American Academy of Neurology Meeting, San Francisco, May 2004.

In embodiments, the MoCA score is determined in a patient 6 hours or later (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more) after a first administration of a therapy described herein, e.g., a natalizumab-like antibody molecule (e.g., natalizumab). In embodiments, a patient has a higher MoCA score after treatment (6 hours or more (e.g., 6, 8, 10, 12, 14, 16, 18, 20, 22, 24 h, 1, 2, 3, 4, 5, 10, 15, 20, 25, 30, 35, 40, 45, 50, 55, 60, 65, 70, 75, 80, 85, 90, 95, or 100 days or more after a first administration of the therapy) compared to before treatment.

In some embodiments, a favorable MoCA outcome after administration of a therapy described herein, e.g., a VLA-4 antagonist, e.g., a natalizumab-like antibody molecule (e.g., natalizumab), is a score greater than or equal to 26. In embodiments, a favorable outcome on the MoCA scale indicates that the therapy is efficacious, e.g., that the patient improved in cognitive function after treatment (e.g., with a treatment described herein, e.g., a natalizumab-like antibody molecule (e.g., natalizumab)), e.g., that the patient has normal function.

In embodiments, a higher MoCA score after administration of a therapy described herein, e.g., a VLA-4 antagonist, e.g., a natalizumab-like antibody molecule (e.g., natalizumab), compared to at baseline, indicates improvement, e.g., that the patient improved in cognitive function after treatment.

Safety/Tolerability

In embodiments, a subject is evaluated for one or more adverse effects after administration of a therapy described herein, e.g., a VLA-4 antagonist, e.g., a natalizumab-like antibody molecule (e.g., natalizumab). Exemplary adverse effects include pneumonia, aspiration pneumonia, urinary tract infection, respiratory tract infection, anemia, and death.

In embodiments, administration of a VLA-4 antagonist, e.g., a natalizumab-like antibody molecule (e.g., natalizumab), leads to minimal adverse effects, e.g., no adverse effects.

In embodiments, a dosage of a VLA-4 antagonist, e.g., a natalizumab-like antibody molecule (e.g., natalizumab), administered to the subject is chosen to reduce or avoid production of antibodies against the VLA-4 binding antibody and/or to prevent an increase the level of circulating white blood cells.

Additionally provided are methods of treating stroke by administering a VLA-4 antagonist in combination with a second therapy, e.g., thrombolysis (e.g., tissue plasminogen activator (tPA)), thrombectomy, angioplasty and stenting, therapeutic hypothermia, and/or a medication (e.g., aspirin, clopidogrel and dipyridamole). In preferred embodiments, the second therapy is, e.g., a thrombolytic agent, a neuroprotective agent, an anti-inflammatory agent, a steroid, a cytokine or a growth factor. The thrombolytic agent used can be tissue plasminogen activator or urokinase. The neuroprotective agent used can be an agonist to a receptor selected from the group consisting of: N-Methyl-D aspartate receptor (NMDA), α-amino-3-hydroxy-5-methyl-4-isoxazoleproprionic acid receptor (AMPA), glycine receptor, calcium channel receptor, bradykinin B2 receptor and sodium channel receptor, or from the group consisting of: the bradykinin B1 receptor, α-amino butyric acid (GABA) receptor, and Adenosine A1 receptor. Anti-inflammatory agents for use can be interleukin-1 and tumor necrosis factor family members.

### Kits

A VLA-4 antagonist described herein may be provided in a kit. The kit includes a VLA-4 antagonist described herein and, optionally, a container, a pharmaceutically acceptable carrier and/or informational material. The informational material can be descriptive, instructional, marketing or other material that relates to the methods described herein and/or the use of the α4 antagonist for the methods described herein.

The informational material of the kits is not limited in its form. In one embodiment, the informational material can include information about production of the VLA-4 antagonist, physical properties of the α4 antagonist, concentration, date of expiration, batch or production site information, and so forth. In one embodiment, the informational material relates to methods for administering the VLA-4 antagonist, e.g., by a route of administration described herein and/or at a dosage and/or dosing schedule described herein.

In one embodiment, the informational material can include instructions to administer a VLA-4 antagonist described herein in a suitable manner to perform the methods described herein, e.g., in a suitable dosage, dosage form, or mode of administration (e.g., a dosage, dosage form, or mode of administration described herein). In another embodiment, the informational material can include instructions to administer a VLA-4 antagonist to a suitable subject, e.g., a human, e.g., a human having a stroke, e.g., within 12 hours or less, e.g., 12, 11, 10, 9 hours or less, e.g., 8, 7, 6, 5 or less hours after LKN.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about an α4 antagonist described herein and/or its use in the methods described herein. The informational material can also be provided in any combination of formats.

In addition to an α4 antagonist, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a pharmaceutically acceptable carrier and/or a second agent for treating a condition or disorder described herein. Alternatively, the other ingredients can be included in the kit, but in different compositions or containers than an α4 antagonist described herein.

In some embodiments, a component of the kit is stored in a sealed vial, e.g., with a rubber or silicone closure (e.g., a polybutadiene or polyisoprene closure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A VLA-4 antagonist described herein can be provided in any form, e.g., liquid, frozen, dried or lyophilized form. It is preferred that a composition including the VLA-4 antagonist described herein be substantially pure and/or sterile. When a VLA-4 antagonist described herein such as a natalizumab-like antibody molecule (e.g., natalizumab) is provided in a liquid solution, the liquid solution preferably is an aqueous solution, with a sterile aqueous solution being preferred. In one embodiment, the VLA-4 antagonist is supplied with a diluents or instructions for dilution. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a VLA-4 antagonist described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. The containers of the kits can be air tight, waterproof (e.g., impermeable to changes in moisture or evaporation), and/or light-tight.

The invention is further illustrated by the following examples, which should not be construed as further limiting.

### EXAMPLES

### Example 1. Effect of Natalizumab in Acute Ischemic Stroke

A Phase 2 clinical trial (“ACTION”) was performed to examine the effect of natalizumab in acute ischemic stroke (AIS). Preclinical data suggest that blocking the α4 subunit of VLA-4 reduces infarct size, thereby improving clinical outcomes [Becker 2001; Relton 2001]. Mononuclear cell infiltration that would be blocked by natalizumab is thought to be a later phenomenon in acute stroke, peaking days after the infarct. However, experimental studies have explored limited time windows after the onset of ischemia. The 6-hour time window for intervention in this study is based on findings from a rodent model of stroke [Hoyte 2010] as well as the time course of soluble VCAM-1 in stroke patients [Lynch 2004], showing that VCAM-1 is increased within 6 hours of stroke onset. Elevated levels of soluble VCAM-1 are also associated with increased risk for recurrent stroke [Castillo 2007]. Natalizumab decreases migration of lymphocytes into the peri-infarct region by blocking the alpha-4 subunit of VLA4. Reducing lymphocyte migration into the peri-infarct region increases the probability that injured neurons survive. Also, promoting survival of injured neurons may improve clinical outcomes. Accordingly, the trial tested whether natalizumab treatment after brain ischemia would reduce post-ischemic inflammation, resulting in smaller infarct volumes and improving clinical outcomes after stroke.

### Protocol

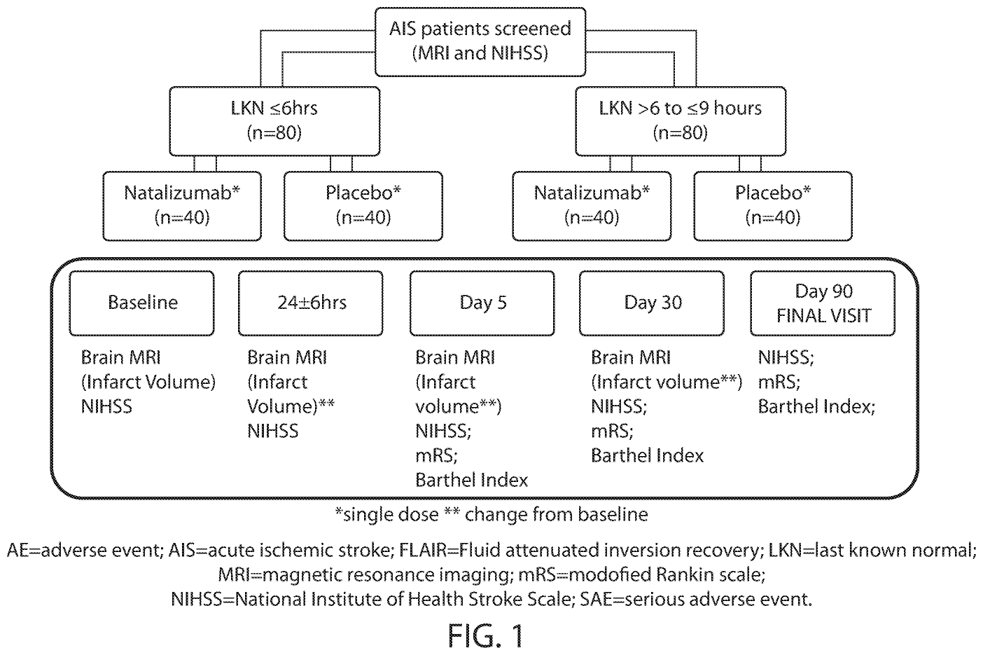

Acute ischemic stroke (AIS) patients were screened by magnetic resonance imaging (MRI) and National Institutes of Health Stroke Scale (NIHSS). At the screening stage, eligibility, demographics, medical history, physical and neurological examination were assessed. At baseline, vital signs, hematology, blood chemistry, pharmacodynamics (PD) serum biomarkers, PD cell phenotype/flow cytometry, anti-JCV antibody status, NIHSS, and brain MRI (infarct volume) were assessed. At 0 hrs, the patient's stroke subtype was classified. One group of patients had a last known normal (LKN) of <6 hours (n=80). Another group of patients had a LKN of <6 to ≤9 hours (n=80). See FIG. 1.

Each group was treated with a single dosage of either natalizumab (single intravenous 300 mg dosage) or placebo. There were 40 patients in each treatment group. Patients were evaluated for endpoints at several timepoints during the study—baseline, 24+/−6 hrs, day 5, day 30, and day 90. At baseline, patients were evaluated by brain MRI (to measure infarct volume) and NIHSS (a neurologic exam used to quantify stroke severity). At 24+/−6 hrs, patients were assessed by physical and neurological examination, e.g., evaluated by brain MRI (to measure infarct volume change from baseline) and NIHSS, as well as hematology for PD/PK (pharmacokinetics). At days 5 and 30, patients underwent physical, neurological, and vital sign examinations, e.g., they were evaluated by brain MRI (infarct volume change from baseline), NIHSS, modified Rankin Scale (mRS), Barthel index, Montreal Cognitive Assessment (MoCA), and Stroke Impact Scale-16 (SIS-16) test, as well as hematology for PD/PK and presence of anti-natalizumab antibodies. On day 90+/−5 days, patients underwent physical, neurological, and vital sign examinations, e.g., they were evaluated by NIHSS, mRS, Barthel index, MoCA, and SIS-16 test, as well as hematology for PD/PK and presence of anti-natalizumab antibodies. See FIG. 1.

Several endpoints were assessed during the study. MRI was used to measure infarct volume. NIHSS, mRS and Barthel index were used to measure functional outcomes. Hematology and blood chemistry and vital sign assessment were used to monitor the patient throughout the trial. Adverse events (AE)/SAE were collected throughout the study. The primary endpoint was change in infarct volume growth from baseline to day 5 (determined by imaging—MRI). Secondary endpoints included clinical endpoints and safety and tolerability. Clinical endpoints included the modified Rankin Scale (mRS) that measures functional independence, the NIHSS scale that measures bedside neurologic function, Barthel's index that measures activities of daily living, stroke-impact scale 16 (SIS-16) that measures patient perceived physical impact of stroke, and the Montreal Cognitive Assessment that measures cognitive ability. The odds ratios and confidence intervals (CI) were derived from a logistic regression model, adjusting for log Baseline diffusion weighted imaging (DWI) volume (<median versus ≥median), treatment time window, location of stroke, and tPA use).

Subgroups of patients were also evaluated during the study—patients with tissue plasminogen activator (tPA) use or without, patients having a lesion size of 2-4 cm or >4 cm at baseline, and patients treated within 0-6 hours after LKN or within 6-9 hours of LKN. Patients with tPA use were those that received tPA within a few hours of receiving natalizumab. The modified intent-to-treat (MITT) subgroup included all subjects randomized and have received the entire infusion of study treatment. The per protocol (PP) subgroup included all subject randomized, have received the entire infusion of study treatment, have no major I/E criteria violations, and no non-petechial hemorrhagic transformation of stroke or ICH.

Subjects were generally comparable on baseline characteristics. See Table 2.

TABLE 2
Baseline Demographics

| Baseline characteristics | Placebo | Natalizumab |
|---|---|---|
| Age, Mean (SD) | 71.6 (11.83) | 70.4 (13.45) |
| Gender (male) | 48/82 (59%) | 39/77 (51%) |
| Weight (kg), Mean (SD) | 75.6 (16.36) | 77.8 (16.82) |
| Baseline DWI volume in mL, | 39.95 (38.936) | 41.52 (45.575) |
| Mean (SD) | | |
| Baseline NIHSS, Mean (SD) | 13.2 (5.84) | 12.9 (6.04) |
| Mean pre-morbid mRS, | 0.9 (1.45) | 0.5 (1.00) |
| Mean (SD) | | |
| tPA use | 65/82 (79%) | 56/77 (73%) |
| Time from LKN to study | 6.40 (1.677) | 6.49 (1.545) |
| drug in hours, Mean (SD) | | |

### Results

Efficacy Endpoint: Imaging

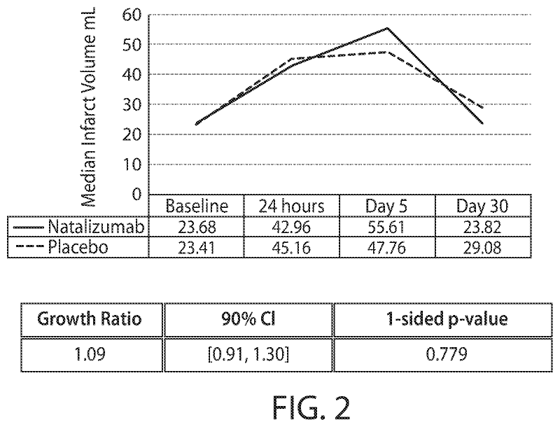

The primary endpoint in the study was imaging (MRI) to detect a change in infarct volume growth from baseline to day 5. Natalizumab did not affect infarct volume growth as defined by MRI. See FIG. 2.

Efficacy Endpoint: Clinical Outcomes

Modified Rankin Scale: Functional Independence

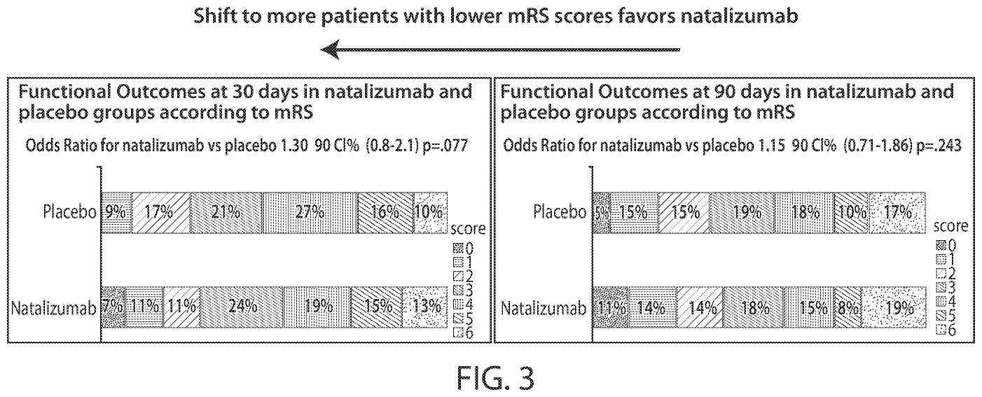

One of the secondary endpoints was the modified Rankin Scale (mRS) score, which is a measure of functional independence. A lower score indicates more functional independence or less disability. mRS functional outcomes at 30 days after treatment or 90 days after treatment were evaluated. A shift to more patients with lower mRS scores indicates natalizumab efficacy. Natalizumab significantly improved functional outcomes at days 30 and 90 on the mRS. See FIG. 3. As shown in Table 3, natalizumab resulted in a trend to greater independence at days 30 and 90 on mRS (global shift of mRS, i.e., improvement relative to baseline). An odds ratio of less than 1 favors placebo, while an odds ratio of greater than 1 favors natalizumab.

TABLE 3
Odds ratios based on mRS scores of natalizumab treated patients
on various days

| | | 90% Confidence | |
|---|---|---|---|
| Visit | Odds ratio | Interval | 1-sided p-value |
| Day 5 | 0.89 | [0.55, 1.45] | 0.564 |
| Day 30 | 1.30 | [0.80, 2.10] | 0.077 |
| Day 90 | 1.15 | [0.71, 1.86] | 0.243 |

The odds ratio of patients with mRS scores of 0 or 1 (excellent outcome) was determined. Table 4 shows the percent of patients (MITT subgroup) in the placebo or natalizumab group having a mRS score of 0 or 1 (excellent outcome) and the odds ratios at days 5, 30 and 90 after treatment. An odds ratio of less than 1 favors placebo, while an odds ratio of greater than 1 favors natalizumab. Natalizumab increased the proportion of patients achieving an excellent outcome (0,1) on the mRS.

TABLE 4
Proportion of patients achieving an excellent mRS outcome - MITT

| | | | | | 1-sided |
|---|---|---|---|---|---|
| mRS (0,1) | Placebo | Natalizumab | OR | 90% CI | p-value |
| Day 5 | 4% (3/82) | 5% (4/76) | 1.46 | [0.40, 5.29] | 0.313 |
| Day 30 | 9% (7/81) | 18% (13/72) | 2.88 | [1.20, 6.93] | 0.024 |
| Day 90 | 21% (16/78) | 25% (18/72) | 1.48 | [0.74, 2.98] | 0.178 |

The odds ratio of a subset of patients having an infarct volume less than median at baseline was determined. Median infarct size (4.6 cm in diameter) is the typical infarct size for stroke clinical trials. Patients having largest infarcts was excluded from this subset. This subset of patients having an infarct volume less than median at baseline had excellent outcomes on mRS (scores of 0,1). Table 5 shows the odds ratios based on mRS for this subset of patients.

TABLE 5

| | | | | 90% Confidence |
|---|---|---|---|---|
| Visit | Placebo | Natalizumab | Odds ratio | Interval |
| Day 5 | 1/42 (2) | 4/37 (11) | 4.97 | [0.76, 32.53] |
| Day 30 | 3/42 (7) | 12/36 (33) | 7.84 | [2.39, 25.76] |
| Day 90 | 11/41 (27) | 15/34 (44) | 2.57 | [1.08, 6.12] |

Barthel Index (BI): Activities of Daily Living

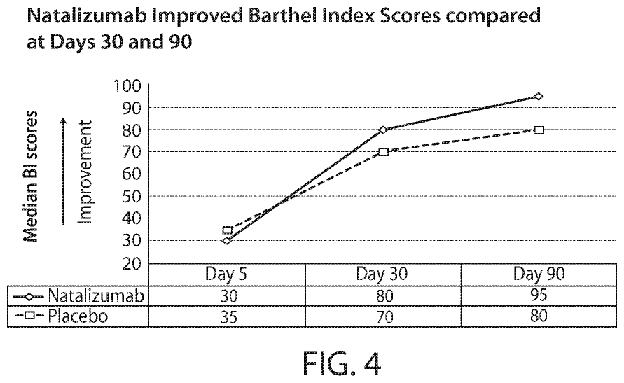

BI scores in natalizumab and placebo groups were compared at various time points. Tables 6 and 7 show the proportion of patients (MITT subgroup) achieving an excellent outcome (BI≥95) and a favorable outcome (BI≥85), respectively. As shown in FIG. 4, natalizumab increased the proportion of patients achieving an excellent outcome (BI≥95) on the BI.

TABLE 6
Proportion of patients achieving excellent outcome - MITT population

| | | | Odds | 2 sided | |
|---|---|---|---|---|---|
| BI (≥95) | Placebo | Natalizumab | Ratios | 90% CI | P-value |
| Day 5 | 15% (12/82) | 14% (11/77) | 0.87 | (0.40, 1.89) | 0.617 |
| Day 30 | 32% (26/82) | 34% (26/77) | 1.13 | (0.63, 2.0) | 0.368 |
| Day 90 | 33% (26/80) | 44% (34/77) | 1.91 | (1.07, 3.41) | 0.033 |

TABLE 7
Proportion of patients achieving favorable outcome - MITT population

| BI (≥85) | Placebo | Natalizumab | Odds Ratios | 2 sided CI | P-value |
|---|---|---|---|---|---|
| Day 5 | 18% (15/82) | 25% (19/77) | 1.48 | (0.75, 2.91) | 0.169 |
| Day 30 | 33% (27/82) | 40% (31/77) | 1.45 | (0.82, 2.55) | 0.142 |
| Day 90 | 38% (30/80) | 45% (35/77) | 1.53 | (0.87, 2.67) | 0.107 |

Odds ratios based on BI scores were determined for the subset of patients having an infarct volume less than median at baseline, where median is a typical infarct size for stroke clinical trials (4.6 cm in diameter) and excludes largest infarcts. Table 8 shows the proportion of this subset of patients having an excellent outcome by BI (a BI score≥95) and the odds ratios.

TABLE 8
Subset of patients having infarct volume < median at
baseline—OR based on BI scores

| Visit | Placebo | Natalizumab | OR (95% CI) |
|---|---|---|---|
| Day 5 | 20% (8/41) | 22% (8/37) | 1.09 (0.42-2.8) |
| Day 30 | 37% (14/38) | 56% (18/32) | 2.70 (1.2-6.0) |
| Day 90 | 42% (14/33) | 68% (19/28) | 3.22 (1.4-7.3) |

NIHSS: Bedside Neurologic Function

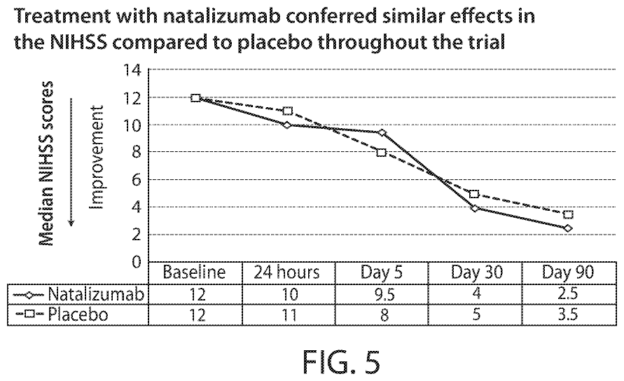

NIHSS scores were compared in natalizumab and placebo groups at various time points. Natalizumab was no different from placebo in reducing overall change from baseline to days 30 and 90 on the proportion of subject with an excellent outcome (0,1) on the NIHSS. See FIG. 5. The proportion of subjects with an excellent outcome on the NIHSS (score of 0, 1 or at least 8 points lower than baseline score) is shown in Table 9. Table 9 also shows the odds ratios, where a ratio of less than 1 favors placebo, and a ratio of greater than 1 favors natalizumab.

TABLE 9
Proportion of subjects with an excellent outcome on NIHSS

| | | | Odds | | 1-sided |
|---|---|---|---|---|---|
| Visit | Placebo | Natalizumab | ratio | 90% CI | p-value |
| 24 Hours | 10/82 (12%) | 9/77 (12%) | 0.99 | [0.43, 2.27] | 0.505 |
| Day 5 | 17/82 (21%) | 14/77 (18%) | 0.87 | [0.44, 1.73] | 0.629 |
| Day 30 | 36/82 (44%) | 27/77 (35%) | 0.69 | [0.39, 1.21] | 0.861 |
| Day 90 | 37/80 (46%) | 36/77 (47%) | 1.10 | [0.63, 1.93] | 0.389 |

Odds ratios based on NIHSS scores were determined for the subset of patients having infarct lesion sizes (volumes) less than median at baseline and are shown in Table 10.

TABLE 10
Subset of patients having lesions less than median at
baseline—odds ratios (OR) based on NIHSS scores

| | Placebo | Natalizumab |
|---|---|---|
| | Median | Median |
| Visit | (Q1, Q3) | (Q1, Q3) |
| Baseline | 11 | 9 |
| | (7, 17) | (6, 12) |
| 24 hours | 9.5 | 7 |
| | (6, 13) | (3, 10) |
| Day 5 | 5 | 5 |
| | (2, 14) | (3, 10) |
| Day 30 | 3 | 3 |
| | (1, 10) | (1, 6) |
| Day 90 | 2 | 1 |
| | (0, 7) | (1, 6) |

Stroke Impact Scale-16 (SIS-16): Patient Perceived Physical Impact of Stroke; and Montreal Cognitive Assessment: Cognitive Ability

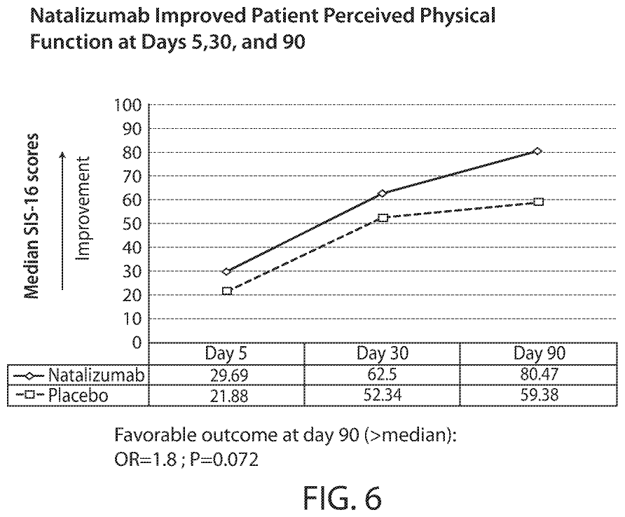

SIS-16 scores in natalizumab and placebo groups were compared at various times point. Natalizumab improved patient perceived physical function at days 5, 30 and 90. A favorable outcome at 90 days (greater than median) corresponds to an odds ratio (OR) of 1.8, p=0.072. See FIG. 6.

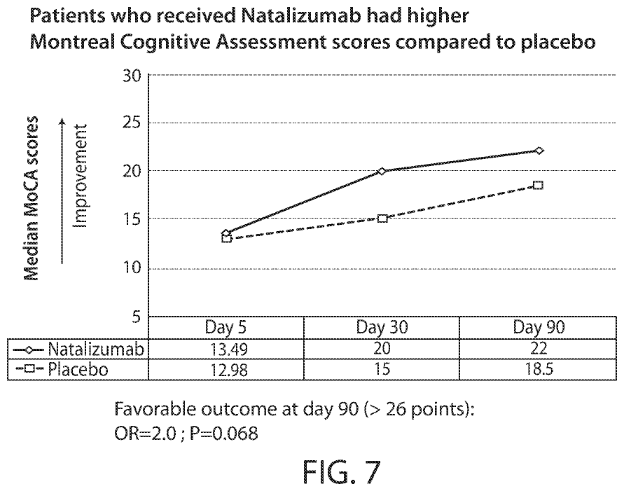

Montreal Cognitive Assessment (MoCA) scores in natalizumab and placebo groups were compared at various time points. Patients who received natalizumab had higher MoCA scores compared to placebo. A favorable outcome at 90 days (greater than 26 points) corresponds to an OR or 2.0, p=0.068. See FIG. 7.

A summary of the clinical endpoint results is shown in Table 11. Overall, clinical endpoint results supported a clinically meaningful benefit of natalizumab at days 30 and 90.

TABLE 11
Summary of clinical endpoint results (NA = not available)

| | | | t-PA 3-4.5 h |
|---|---|---|---|
| | Odds Ratio for | Odds Ratio for | Reference Odds |
| | Favorable | Favorable | Ratio from |
| Clinical | Outcome at Day | Outcome at Day | ECASS3 Trial |
| outcome scale | 30 (95% CI) | 90 (95% CI) | at Day 90 |
| Secondary Endpoints | | | |
| mRS score of 0 or 1 | 2.95 (0.94, 9.27) | 1.50 (0.59, 3.81) | 1.34 (1.02-1.76) |
| Barthel Index ≥ 95 | 1.10 (0.49, 2.46) | 2.12 (0.95, 4.72) | 1.23 (0.93-1.62) |
| NIHSS score 0 or 1 | 0.75 (0.30-1.84) | 0.82 (0.36, 1.89) | 1.33 (1.01-1.75) |
| Global OR | 1.32 (0.70, 2.48) | 1.37 (0.73, 2.57) | 1.28 (1.00-1.65) |
| Tertiary endpoints | | | |
| Stroke Impact | 1.09 (0.59-2.02) | 1.80 (0.93-3.49) | NA |
| Scale—16 > median | | | |
| Montreal Cognitive | 1.05 (0.40-2.73) | 2.03 (0.93-4.46) | NA |
| Assessment ≥ 26 | | | |

Composite Outcomes

Intent-to-Treat Population

The global or composite clinical outcomes/endpoints of patients in the intent-to-treat population were calculated at day 30 and day 90. (The intent-to-treat subgroup included all patients including those who did not complete the trial; the per protocol subgroup consisted of patients who completed the trial.) A global outcome odds ratio (OR) was calculated based on the proportion of patients having excellent mRS, BI, and NIHSS scores. Table 12 shows the proportion of patients having a mRS score of 0,1, BI score of ≥95, and NIHSS score of 0.1 at 30 days, as well as the calculated OR's for each endpoint and the OR for global outcome. Table 13 shows the proportion of patients having a mRS score of 0,1, BI score of ≥95, and NIHSS score of 0.1 at 90 days, as well as the calculated OR's for each endpoint and the OR for global outcome. The OR's were adjusted for age and baseline NIHSS score.

TABLE 12
Odds ratios for clinical endpoints* in the intent-to-treat
population at day 30 (age and baseline NIHSS score adjusted)

| | Natalizumab | Placebo | Odds Ratio | One-sided |
|---|---|---|---|---|
| | (N = 77) | (N = 82) | (95% CI) | P Value |
| Global outcome^{a} | | | 1.32 (0.70, 2.48) | 0.192 |
| mRS score of | 13 (17.8) | 7 (8.6) | 2.95 (0.94, 9.27) | 0.033 |
| 0 or l^{b} | | | | |
| Barthel Index | 26 (33.8) | 26 (32.1) | 1.10 (0.49, 2.46) | 0.409 |
| Score ≥ 95^{c} | | | | |
| NIHSS score of | 14 (18.2) | 18 (22.0) | 0.75 (0.30, 1.84) | 0.764 |
| 0 or 1 ^{d} | | | | |

*Missing values (including deaths) were imputed per analysis plan.
^{a}The global outcome analysis is a multidimensional calculation of a favorable outcome defined by the outcome in each individual scale. The statistical approach is a global odds ratio test based on a linear logistic regression model (using generalized estimation equations to perform a Wald-type test) with TPA use, time to treatment, age category (≤80, >80), log baseline infarct volume (DWI), and baseline NIHSS category (<=12, >12; 12 is median).
^{b}Scores on the modified Rankin scale range from 0 (no symptoms at all) to 6 (death). The analysis was based on logistic regression with the same covariates as above.
^{c}The Barthel index assesses the ability to perform activities of daily living on a scale that ranges from 0 (complete dependence on help with activities of daily living) to 100 (independence). The analysis was based on logistic regression with the same covariates as above.
^{d} Scores on NIHSS range from 0 to 42, with higher values reflecting more severe neurologic impairment (<5, mild impairment; ≥25, very severe impairment). The analysis was based on logistic regression with the same covariates as above.

TABLE 13
Odds ratio for clinical endpoints* in the intent-to-treat
population at day 90 (age and baseline NIHSS score adjusted)

| | | | | One- |
|---|---|---|---|---|
| | Natalizumab | Placebo | Odds Ratio | sided |
| | (N = 77) | (N = 82) | (95% CI) | P Value |
| Global outcome^{a} | | | 1.37 (0.73, 2.57) | 0.163 |
| mRS score of | 18 (24.7) | 16 (20.5) | 1.50 (0.59, 3.81) | 0.198 |
| 0 or 1^{b} | | | | |
| Barthel Index | 34 (44.2) | 26 (32.9) | 2.12 (0.95, 4.72) | 0.033 |
| Score ≥ 95^{c} | | | | |
| NIHSS score of | 20 (26.0) | 22 (27.5) | 0.82 (0.36, 1.89) | 0.822 |
| 0 or 1^{d} | | | | |

*Missing values (including deaths) were imputed per analysis plan.
^{a}The global outcome analysis is a multidimensional calculation of a favorable outcome defined by the outcome in each individual scale. The statistical approach is a global odds ratio test based on a linear logistic regression model (using generalized estimation equations to perform a Wald-type test) with TPA use, time to treatment, age category (≤80, >80), log baseline infarct volume (DWI), and baseline NIHSS category (<=12, >12; 12 is median).
^{b}Scores on the modified Rankin scale range from 0 (no symptoms at all) to 6 (death). The analysis was based on logistic regression with the same covariates as above.
^{c}The Barthel index assesses the ability to perform activities of daily living on a scale that ranges from 0 (complete dependence on help with activities of daily living) to 100 (independence). The analysis was based on logistic regression with the same covariates as above.
^{d}Scores on NIHSS range from 0 to 42, with higher values reflecting more severe neurologic impairment (<5, mild impairment; ≥25, very severe impairment). The analysis was based on logistic regression with the same covariates as above.

Baseline Infarct Size<4.6 cm in Diameter

The odds ratios for a subset of the intent-to-treat patients—those having a baseline infarct size <median (<4.6 cm in diameter)—were determined for day 30 and day 90 based on clinical endpoints. Table 14 shows the day 30 odds ratio for clinical endpoints in this subpopulation. Table 15 shows the day 90 odds ratio for clinical endpoints in this subpopulation. The odds ratios were age and baseline NIHSS score adjusted.

TABLE 14
Day 30 Odds Ratio for Clinical Endpoints* in the Intent-to-treat in the
Sub-population Defined as Baseline Infarct Size < 4.6 cm in diameter

| | | | | One- |
|---|---|---|---|---|
| | Natalizumab | Placebo | Odds Ratio | sided |
| | (N = 37) | (N = 42) | (95% CI) | P Value |
| Global outcome^{a} | | | 1.71 (0.79, 3.67) | 0.086 |
| mRS score of | 12 (33.3) | 3 (7.1) | 6.97 (1.46, 33.38) | 0.008 |
| 0 or 1^{b} | | | | |
| Barthel Index | 20 (54.1) | 14 (34.2) | 1.98 (0.58, 6.69) | 0.137 |
| Score ≥ 95^{c} | | | | |
| NIHSS score of | 11 (29.7) | 14 (33.3) | 0.55 (0.17, 1.74) | 0.654 |
| 0 or 1 ^{d} | | | | |

*Missing values (including deaths) were imputed per analysis plan.
^{a}The global outcome analysis is a multidimensional calculation of a favorable outcome defined by the outcome in each individual scale. The statistical approach is a global odds ratio test based on a linear logistic regression model (using generalized estimation equations to perform a Wald-type test) with TPA use, time to treatment, age category (≤80, >80), log baseline infarct volume (DWI), and baseline NIHSS category (<=12, >12; 12 is median).
^{b}Scores on the modified Rankin scale range from 0 (no symptoms at all) to 6 (death). The analysis was based on logistic regression with the same covariates as above.
cThe Barthel index assesses the ability to perform activities of daily living on a scale that ranges from 0 (complete dependence on help with activities of daily living) to 100 (independence). The analysis was based on logistic regression with the same covariates as above.
^{d} Scores on NIHSS range from 0 to 42, with higher values reflecting more severe neurologic impairment (<5, mild impairment; ≥25, very severe impairment). The analysis was based on logistic regression with the same covariates as above.

TABLE 15
Day 90 Odds Ratio for Clinical Endpoints* in the Intent-to-treat in the
Sub-population Defined as Baseline Infarct Size < 4.6 cm in diameter

| | Natalizumab | Placebo | Odds Ratio | One-sided |
|---|---|---|---|---|
| | (N = 37) | (N = 42) | (95% CI) | P Value |
| Global outcome^{a} | | | 1.36 (0.56, 3.30) | 0.250 |
| mRS score of | 15 (44.1) | 11 (26.8) | 1.98 (0.61, 6.37) | 0.128 |
| 0 or 1^{b} | | | | |
| Barthel Index | 22 (59.5) | 15 (36.6) | 2.60 (0.79, 8.50) | 0.057 |
| Score ≥ 95^{c} | | | | |
| NIHSS score of | 15 (40.5) | 17 (40.5) | 0.68 (0.24, 1.91) | 0.733 |
| 0 or 1^{d} | | | | |

*Missing values (including deaths) were imputed per analysis plan.
^{a}The global outcome analysis is a multidimensional calculation of a favorable outcome defined by the outcome in each individual scale. The statistical approach is a global odds ratio test based on a linear logistic regression model (using generalized estimation equations to perform a Wald-type test) with TPA use, time to treatment, age category (≤80, >80), log baseline infarct volume (DWI), and baseline NIHSS category (<=12, >12; 12 is median).
^{b}Scores on the modified Rankin scale range from 0 (no symptoms at all) to 6 (death). The analysis was based on logistic regression with the same covariates as above.
^{c}The Barthel index assesses the ability to perform activities of daily living on a scale that ranges from 0 (complete dependence on help with activities of daily living) to 100 (independence). The analysis was based on logistic regression with the same covariates as above.
^{d}Scores on NIHSS range from 0 to 42, with higher values reflecting more severe neurologic impairment (<5, mild impairment; ≥25, very severe impairment). The analysis was based on logistic regression with the same covariates as above.

The global outcome from treatment with natalizumab was compared to the 90 day global outcome outcome of a reference treatment, e.g., t-PA at 3-4.5 hours after stroke, which is described in a European Cooperative Acute Stroke Study (ECASS) in Hacke et al. New Engl. J. Med. 359.13(2008):1317-29. In the reference t-PA treatment, in the intent-to-treat population, the odds ratio was 1.34 based on an endpoint of a mRS score of 0 or 1 (by an unadjusted analysis); and the odds ratio was 1.28 based on global outcome (factoring in mRS score of 0,1, BI score of ≥95, NIHSS score of 0,1, and GOS score of 1. See Hacke et al. The odds ratios observed in the natalizumab study described herein were about same as if not higher than (indicative of greater efficacy) those in the reference t-PA treatment. These results suggest that natalizumab when administered at up to 9 hours post stroke had efficacious clinical outcomes on par with (if not better than) the reference t-PA treatment administered at 3-4.5 hours post stroke.

Safety/Tolerability

Safety and tolerability were monitored during the study. A shown in Table 16, no significant safety or tolerability concerns were identified.

TABLE 16
Safety and tolerability results

| | Placebo | Natalizumab |
|---|---|---|
| | (n = 82) | (n = 78) |
| Incidence of Death | | |
| Death as of: | | |
| Day 5 | 2 (2%) | 3 (4%) |
| Day 30 | 8 (10%) | 9 (12%) |
| Day 90 | 13 (16%) | 14 (18%) |
| Incidence of SAEs | | |
| | 38 (46%) | 36 (46%) |
| Incidence of AEs | | |
| | 81 (99%) | 77 (99%) |
| Pneumonia | 7(9%) | 12 (15%) |
| Aspiration Pneumonia | 4(5%) | 2 (3%) |
| Urinary Tract Infection | 15 (18%) | 19 (24%) |
| Respiratory Tract Infections | 9(11%) | 5(6%) |
| Anemia | 4(5%) | 8(10%) |

Overall, there were 13 deaths in the placebo group, 14 deaths in the natalizumab group, 38 (46%) SAEs in the placebo group, 36 (46%) SAEs in the natalizumab group, 36 (44%) infections in the placebo group, 36 (46%) infections in the natalizumab group, 15 (18%) with depression in the placebo group, and 9 (11%) with depression in the natalizumab group.

Pharmacokinetics (PK)

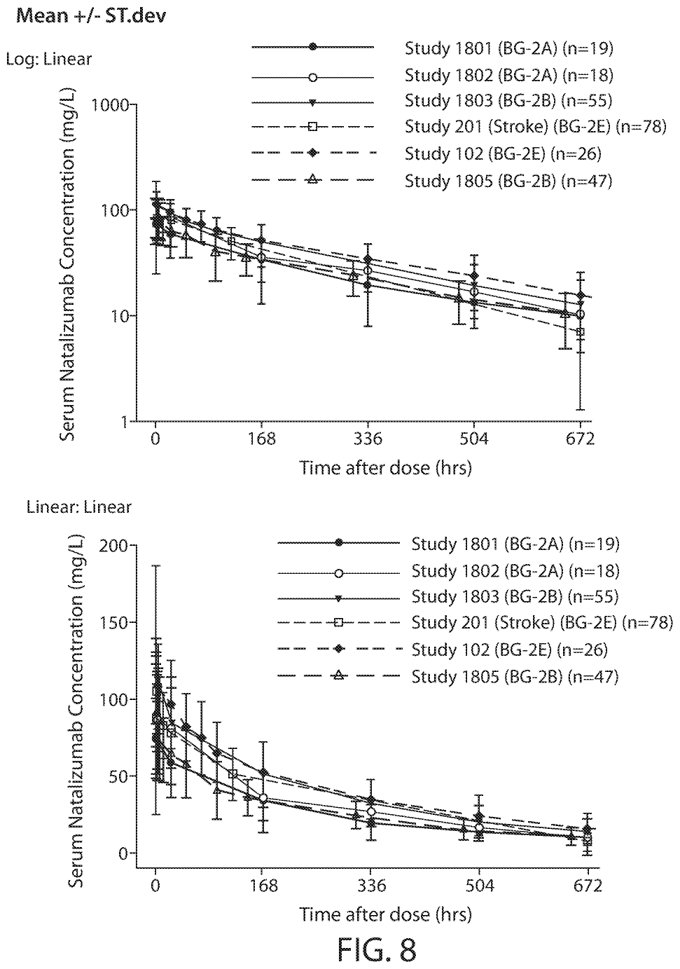

The PK of natalizumab was also examined in the stroke patients and compared to that observed in multiple sclerosis (MS) patients. As shown in FIG. 8, there was an overlap of PK profiles in stroke patients compared to PK profiles in MS patients. Thus, there was no significant difference in PK observed between MS and stroke patients.

### Conclusions

A summary of the results from the endpoint analyses is shown in Table 17.

TABLE 17

| Endpoint | Result |
|---|---|
| Primary | |
| Change in growth from baseline to Day 5 | No effect |
| Secondary | |
| Imaging | |
| Change in Infarct Volume at other time intervals | No effect |
| Clinical | |
| mRS | Effect |
| Continuous and dichotomous | |
| BI | Effect |
| Continuous and dichotomous | |
| NIHSS | No effect |
| Continuous and dichotomous | |
| Exploratory | |
| SIS-16 | Effect |
| Montreal Cognitive Assessment | Effect |

In the Phase 2 trial described herein, the data showed that post-stroke inflammation may be relevant to functional/clinical outcomes after stroke, even if not detected as focal infarct volume expansion on MRI. One possible reason may be that inflammatory injury in stroke (e.g., post-ischemic inflammation) may be too diffuse to be measured by focal lesion volume growth on MRI. Inflammation can occur longer for weeks to months as demonstrated by the presence of Immunoglobulins in the cerebrospinal fluid occurring in weeks to months after the onset of a stroke (see, e.g., Pruss et al. Arch. Neurol. 69.6(2012):714-17). Preclinical evidence suggest that the chronic inflammation may be deleterious (such as cognitive dysfunction) in regions of the brain not contiguous with the infarct lesion (see, e.g., Doyle et al. J. Neurosci. 35.5(2015):2133-45).

The results provide evidence that reducing post-stroke inflammation with a single dosage of natalizumab likely leads to gains in higher order domains such as cognition and likely contributes to functional independent at 90 days after treatment. Receptor saturation was consistent with previous multiple sclerosis studies.

The Phase 2 trial demonstrated that in acute ischemic stroke, natalizumab (as compared to placebo) did not change MRI-defined infarct growth (primary endpoint). Beneficial effects on clinical outcomes were seen on 2 out of 3 secondary endpoints and 4 out of 5 clinical outcome measures overall. The positive clinical effects reflected gains in higher order domains such as cognition and functional independence in daily living. The results herein provide evidence of benefit on modified Rankin scale, which is the most commonly used Phase 3 registrational endpoint in acute stroke. No effect from natalizumab over placebo was seen on neurologic deficits measured by NIHSS. Secondary endpoints measuring clinical outcomes (e.g., mRS and Barthel's index) suggested that natalizumab treatment would improve stroke outcomes compared to placebo. The positive clinical effects were more pronounced in patients with a baseline infarct size less than median (compared to those with a baseline infarct size greater than median). Clinical outcomes (mRS, Barthel Index, NIHSS) were correlated with infarct volume growth in the natalizumab group. No safety or tolerability concerns were identified. The results suggest that natalizumab in acute ischemic stroke has a favorable benefit-risk profile. Natalizumab likely benefits acute stroke patients when given up to 9 hours post stroke.

### Example 2: Exposure Response of Subjects in Phase II Trial

The exposure response of subjects in the Phase II trial described in Example 1 was examined. The natalizumab treated subjects were separated into three tertiles based on their exposure (AUC)—with the first tertile having the lowest AUC and third tertile having the highest AUC. Tertile 1 had an AUC of 6970 to 17340 mg*hr/L. Tertile 2 had an AUC of 17990 to 27580 mg*hr/L. Tertile 3 had an AUC of 27720 to 43300 mg*hr/L. Clinical outcomes at day 90 were compared among the three AUC tertiles in order to assess the relationship between exposure (AUC) and clinical outcome. Table 20 shows the percentage of subjects (in each AUC tertile) having an excellent NIHSS score (0 or 1) on day 90. The percentage of subjects having a NIHSS score of 0 or 1 was higher in the subset of subjects having an AUC in the third tertile (higher AUC). The odds ratio to placebo for this subset of subjects was also greater than the other tertiles (the odds ratio for the third tertile was greater than 1, whereas the other tertiles had odds ratios less than 1).

TABLE 20

| | | Proportion with | Odds Ratio to | P-Value |
|---|---|---|---|---|
| NIHSS (Day 90) | N | NIHSS(0, 1) | Placebo* | |
| Placebo | 80 | 28% | | |
| Natalizumab | 77 | 26% | 0.82 | 0.644 |
| | | | (0.36, 1.89) | |
| Natalizumab with | 25 | 8% | 0.18 | 0.058 |
| AUC in first tertile | | | (0.02, 0.90) | |
| Natalizumab with | 27 | 26% | 0.71 | 0.571 |
| AUC in second tertile | | | (0.21, 2.24) | |
| Natalizumab with | 25 | 44% | 1.78 | 0.325 |
| AUC in third tertile | | | (0.56, 5.72) | |

*adjusted for weight and base me NIHSS

Table 21 shows the percentage of subjects (in each AUC tertile) having an excellent mRS score (0 or 1) on day 90. The percentage of subjects having a mRS score of 0 or 1 was higher in the subset of subjects having an AUC in the third tertile (higher AUC). The odds ratio to placebo for this subset of subjects was greater than the other tertiles (the odds ratio for the third tertile was greater than 1, whereas the other tertiles had odds ratios less than 1).

TABLE 21

| | | Proportion | | |
|---|---|---|---|---|
| | | with | Odds Ratio | |
| mRs (Day 90) | N | mRS(0, 1) | to Placebo* | P-Value |
| Placebo | 78 | 21% | | |
| Natalizumab | 72 | 25% | 1.48 | 0.356 |
| | | | (0.74, 2.98) | |
| Natalizumab with AUC | 21 | 10% | 0.64 | 0.635 |
| in first tertile | | | (0.08, 3.63) | |
| Natalizumab with AUC | 26 | 19% | 0.84 | 0.794 |
| in second tertile | | | (0.21, 3.06) | |
| Natalizumab with AUC | 25 | 44% | 3.02 | 0.079 |
| in third tertile | | | (0.89, 10.75) | |

*adjusted for weight and baseline NIHSS

Table 22 shows the percentage of subjects (in each AUC tertile) having an excellent BI score (≥95) on day 90. The percentage of subjects having a BI score of ≥95 was higher in the subset of subjects having an AUC in the second or third tertiles (higher AUC). The odds ratio to placebo for these subsets of subjects was also greater than for the first tertile (the odds ratio for the second and third tertiles was greater than 1, whereas the first tertile had odds ratios less than 1).

TABLE 22

| | | Proportion | Odds | |
|---|---|---|---|---|
| | | with | Ratio to | |
| Barthel Index—Day 90 | N | BI >= 95 | Placebo* | P-Value |
| Placebo | 80 | 33% | | |
| Natalizumab | 77 | 44% | 1.91 | 0.066 |
| | | | (1.07, 3.41) | |
| Natalizumab with AUC | 25 | 16% | 0.48 | 0.295 |
| in first tertile | | | (0.11, 1.79) | |
| Natalizumab with AUC | 27 | 52% | 2.39 | 0.109 |
| in second tertile | | | (0.83, 7.13) | |
| Natalizumab with AUC | 25 | 64% | 3.00 | 0.062 |
| in third tertile | | | (0.97, 9.99) | |

*adjusted for weight and baseline NIHSS

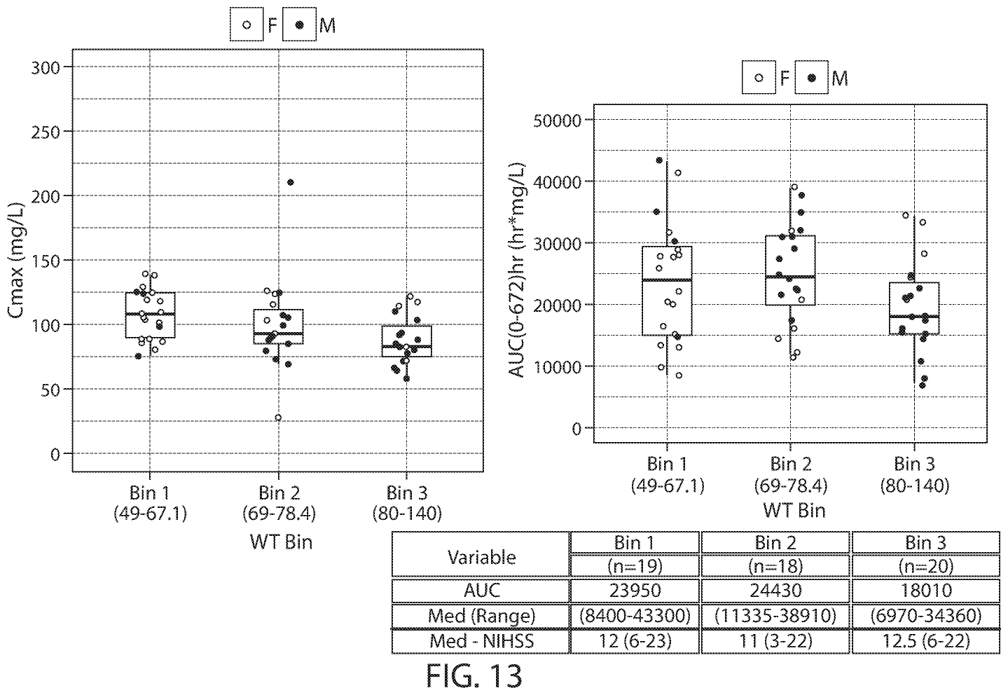

Additionally, the effect of weight on AUC was examined. Patients were binned into 3 bins based on their weight, as shown in FIG. 13, e.g., with separate bins corresponding to weight ranges of 49-67.1 kg (bin 1), 69-78.4 kg (bin 2), and 80-140 kg (bin 3). The Cmax and AUC were compared among the bins of patients (FIG. 13). The AUC (at 30 days) in bin 1 was about 1.3 times that of the AUC in bin 3. Thus, the AUC was higher in low-weight patients than high-weight patients.

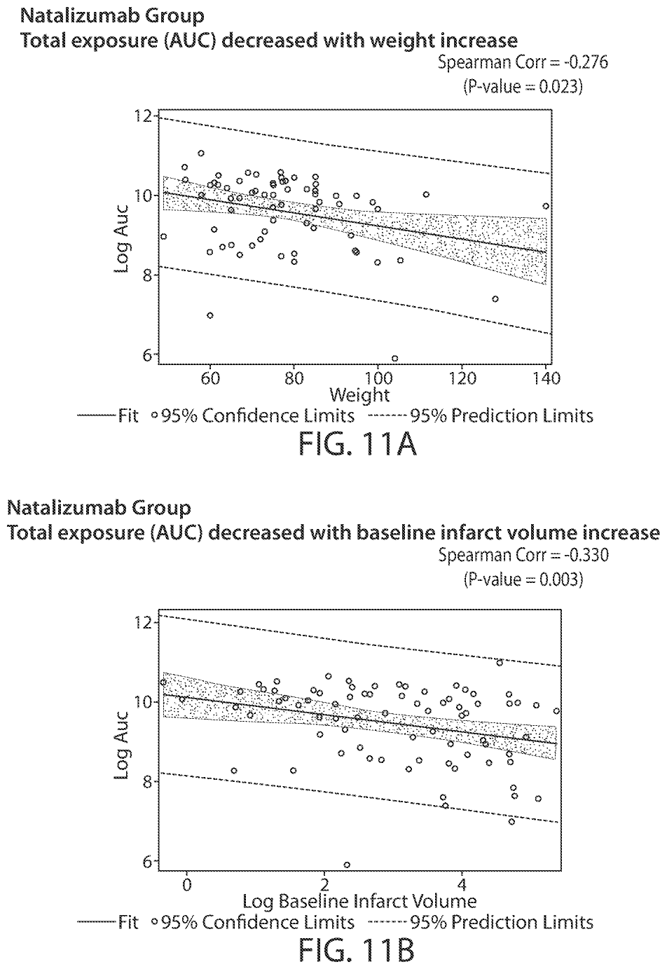

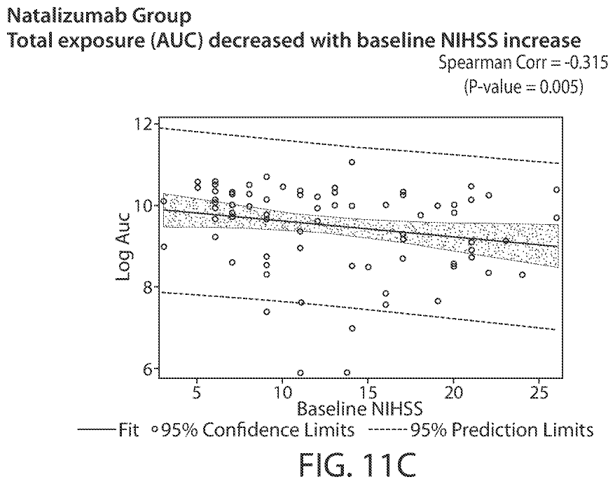

Also, the relationship between stroke severity at baseline and natalizumab exposure was determined. There was an unexpected relationship between infarct size or NIHSS score and natalizumab exposure. Baseline stroke severity was inversely correlated with natalizumab exposure (AUC measured over 30 days) (FIGS. 11A, 11B, and 11C). In natalizumab-treated subjects, total exposure (AUC) decreased with weight increase (FIG. 11A), total exposure (AUC) decreased with baseline infarct volume increase (FIG. 11B), and total exposure (AUC) decreased with baseline NIHSS score increase (FIG. 11C). Table 18 shows the slope values from a plot of AUC (at day 5 or day 30) versus baseline infarct volume.

TABLE 18

| | Slope | SE of | |
|---|---|---|---|
| Infarct Volume Growth—Day 5 | Estimate* | Estimate | P-Value |
| Day 5 | | | |
| AUC (in log scale) | −0.14 | 0.08 | 0.076 |
| Baseline Infarct Volume (in log scale) | −0.11 | 0.06 | 0.065 |
| Day 30 | | | |
| AUC (in log scale) | −0.26 | 0.10 | 0.013 |
| Baseline Infarct Volume (in log scale) | −0.11 | 0.08 | 0.157 |

*adjusted for weight and baseline NIHSS

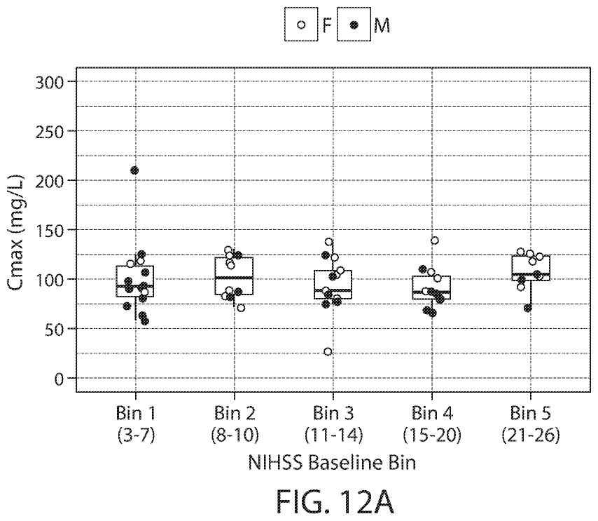

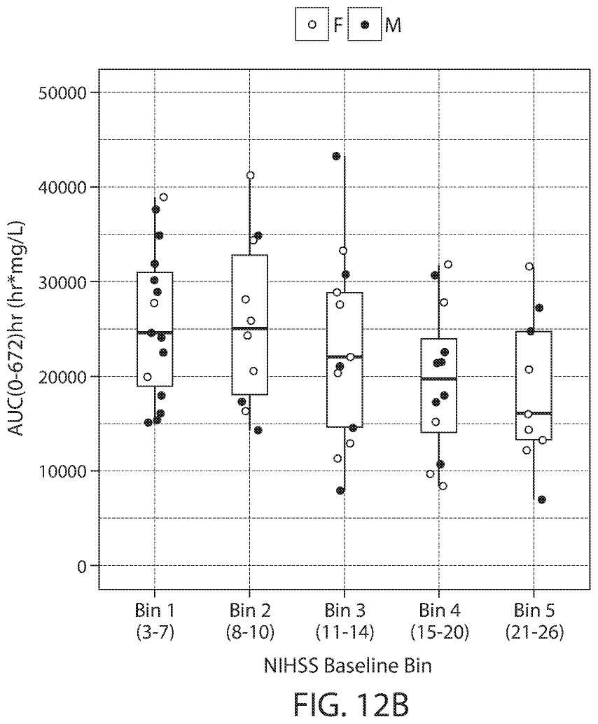

To further assess the effect of baseline stroke severity based on NIHSS on AUC, patients were binned into 5 bins based on their NIHSS scores, as shown in Table 19, e.g., with separate bins corresponding to median NIHSS scores of 6, 9, 13, 17.5, or 22.0. The Cmax and AUC were compared among the bins of patients (FIGS. 12A and 12B). There was no effect of baseline NIHSS on Cmax (FIG. 12A). The median AUC differential between the lowest baseline NIHSS quintile (Binl) and the highest baseline NIHSS quintile (Bin5) was approximately 30% (AUC by non-compartmental analysis) or 21% (AUC by population PK analysis) (FIG. 12B). See also FIG. 11C, which shows that total exposure (AUC) decreased with baseline NIHSS increase. As shown in Table 19, the weight and age distributions across the quintiles/bins were balanced across Bin 1 and 5. Baseline NIHSS was tested as a covariate for AUC in a population PK model, which accepts that baseline NIHSS is related to AUC after accounting for weight.

TABLE 19

| | Bin 1 | Bin 2 | Bin 3 | Bin 4 | Bin 5 |
|---|---|---|---|---|---|
| Variable | (n = 15) | (n = 10) | (n = 13) | (n = 12) | (n = 9) |
| WT | 75.0 | 81.0 | 67.0 | 77.2 | 73.0 |
| Med (Range) | (62.0-140) | (54-95) | (49-95) | (60-112) | (54-105) |
| Age | 71.0 | 73.0 | 71.0 | 77.0 | 79.0 |
| Med (Range) | (44-84) | (44-85) | (19-84) | (45-83) | 62-82 |
| Med—NIHSS | 6 | 9 | 13 | 17.5 | 22.0 |

### Conclusions

The AUC is a measure of exposure to drug, with a higher AUC indicating greater exposure to drug. Higher natalizumab exposure (AUC) was correlated with a better clinical response than lower exposure. Higher exposure was correlated with reduced infarct volume growth on MRI at day 5; higher proportion of subjects with an excellent outcome on the NIHSS (0,1) scores on day 90; mRS scores (0,1) on day 90; and higher BI (95) scores on day 90. The results also show that patients with larger stroke severity at baseline, larger infarct volume at baseline, or higher weight tended to have lower exposure to natalizumab, e.g., over 30 days, with minimal to no impact on Cmax. Patients with higher stroke severity (as categorized by NIHSS) at baseline tended to have about 30% lower drug exposure at a median level relative to those with lower stroke severity at baseline. This suggests that there was a proportion of subjects in the trial with even lower exposures (more than 30% below median level).

The data suggests that the lower AUC observed in some individuals (e.g., some individuals with a high baseline NIHSS (more severe stroke at baseline), a larger infarct size, and/or with a higher weight) can be compensated for by increasing the dosage of natalizumab. For example, increasing the dosage of natalizumab by 30-100%, e.g., 30-50%, above 300 mg, e.g., to a dosage of about 450 mg or about 600 mg, could provide desired clinical outcomes in a majority of individuals (including those with larger infarct volumes at baseline, those with smaller infarct volumes at baseline, and a wide range of body weights, those with severe strokes, and those with less than severe strokes), not just a median individual.

Other embodiments are in the claims.

What is claimed is:

1. A method of treating a human subject having a stroke, e.g., an ischemic stroke, e.g., an acute ischemic stroke, a hemorrhagic stroke, e.g., intracerebral hemorrhage, or a subarachnoid hemorrhage, comprising:
administering a VLA-4 antagonist, e.g., an anti-alpha4 antibody molecule, e.g., a natalizumab-like antibody molecule, e.g., natalizumab, to the subject, at a dosage (e.g., as a single administration) of:
i) 350 to 500, e.g., 390 to 450, or 450+/−5%, or about 450, e.g., 450, mg; 550 to 650, e.g., 575 to 625, e.g., 600+/−5%, e.g., about 600, e.g., 600, mg;
ii) a dosage that results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L; or
iii) a dosage that results in an AUC that is comparable or higher than (e.g., within 5-10% of) the median exposure observed in lower body weight (e.g., body weight of <80 kg) or less severe stroke patients;
thereby treating the human subject.

2. The method of claim 1, wherein the dosage is 350 to 500 mg.

3. The method of claim 1, wherein the dosage is 390 to 450 mg.

4. The method of claim 1, wherein the dosage is 450+/−5% mg.

5. The method of claim 1, wherein the dosage is about 450 mg.

6. The method of claim 1, wherein the dosage is 450 mg.

7. The method of claim 1, wherein the dosage is 550 to 650 mg.

8. The method of claim 1, wherein the dosage is 575 to 625 mg.

9. The method of claim 1, wherein the dosage is 600+/−5% mg.

10. The method of claim 1, wherein the dosage is about 600 mg.

11. The method of claim 1, wherein the dosage is 600 mg.

12. The method of claim 1, wherein the subject has a severe stroke, e.g., a stroke having a NIHSS score equal to or greater than 15 or 21.

13. The method of claim 1, wherein the subject has a stroke having a NIHSS score equal to or greater than 15.

14. The method of claim 1, wherein the subject has a stroke having a NIHSS score equal to or greater than 21.

15. The method of claim 1, wherein the subject has a less than severe stroke, e.g., a stroke having a NIHSS score of less than 15.

16. The method of claim 1, wherein the subject, at baseline, has an infarct size equal to or greater than 4.6 cm in diameter.

17. The method of claim 1, wherein the subject, at baseline, has an infarct size less than 4.6 cm in diameter.

18. The method of claim 1, comprising:
determining, e.g., determining prior to administration of the dosage, the severity of the stroke, e.g., determining if the patient has a severe stroke.

19. The method of claim 18, wherein determining comprises determining a NIHSS score, e.g., determining if the NIHSS score is equal to or greater than 15 or 21.

20. The method of claim 18, wherein determining comprises determining if the NIHSS score is equal to or greater than 15.

21. The method of claim 18, wherein determining comprises determining if the NIHSS score is equal to or greater than 21.

22. The method of claim 18, wherein determining comprises determining a NIHSS score, e.g., determining if the NIHSS score is less than 15.

23. The method of claim 18, wherein responsive to the determination of severity, a dosage of anti-VLA4 antagonist is selected.

24. The method of claim 1, wherein administration of the dosage is initiated within 6 hours of last known normal.

25. The method claim 1, wherein administration of the dosage is initiated within 9 hours, e.g., 6 to 9 hours, of last known normal.

26. The method of claim 1, wherein administration of the dosage is initiated within 12 hours, e.g., 6 to 12 hours, or 9 to 12 hours, of last known normal.

27. The method of claim 1, further comprising administering a subsequent dosage of the VLA-4 antagonist.

28. The method of claim 27, wherein the subsequent dosage is 125 to 175, 150+/−5%, about 150, or 150, mg.

29. The method of claim 27, wherein the subsequent dosage is 150 mg.

30. The method of claim 27, wherein the subsequent dosage is 250 to 350, or 300+/−5%, e.g., about 300, e.g., 300, mg.

31. The method of claim 27, wherein the subsequent dosage is 300 mg.

32. The method of claim 27, wherein the subsequent dosage is 350 to 500, e.g., 390 to 450, or 450+/−5%, or about 450, e.g., 450, mg.

33. The method of claim 27, wherein the subsequent dosage is 450 mg.

34. The method of claim 27, wherein the subsequent dosage is 550 to 650, e.g., 575 to 625, e.g., 600+/−5%, e.g., about 600, e.g., 600, mg.

35. The method of claim 27 wherein the subsequent dosage is 600 mg.

36. The method claim 27, wherein the subsequent dosage is administered on day 3 after the dosage (e.g., if the dosage is administered on the first day of the month the subsequent dosage is administered on the third day of the month.

37. The method of claim 27, wherein the subsequent dosage is administered on day 5 after the dosage.

38. The method of claim 27, wherein the subsequent dosage is administered on day 7 after the dosage.

39. The method of claim 27, wherein the subsequent dosage is administered four to six weeks after the dosage.

40. The method of claim 1, wherein the VLA-4 antagonist comprises and antibody molecule that comprises CDR1, CDR2 and CDR3 from the light chain and CDR1, CDR2 and CDR3 from the heavy chain of natalizumab.

41. The method of claim 1, wherein the VLA-4 antagonist comprises and antibody molecule that comprises the light chain variable region and the heavy chain variable region of natalizumab.

42. The method of claim 1, wherein the VLA-4 antagonist comprises natalizumab.

43. The method of claim 1, comprising administering a VLA-4 antagonist to the subject within 12 hours or less, e.g., 10, 9, 8, 7, 6 hours or less, after LKN in the subject.

44. The method of claim 1, wherein the VLA-4 antagonist is administered within 9 hours or less after LKN, e.g., within 9, 8, 7, or 6 hours or less after LKN, or between 6 and 9 hours after LKN, or within 6 hours after LKN.

45. The method of claim 1, wherein the VLA-4 antagonist is administered within 6 hours or less after LKN, e.g., between 3 and 6 hours, 4.5 to 6 hours, 5 to 6 hours, after LKN.

46. The method of claim 1, comprising administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject.

47. The method of claim 1, wherein the VLA-4 antagonist is administered within more than 2 to 9 hours or less after LKN, e.g., between 6 and 9 hours after LKN.

48. The method of claim 1, wherein the VLA-4 antagonist is administered within more than 2 hours to 6 hours or less after LKN, e.g., between 3 and 6 hours, 4.5 to 6 hours, 5 to 6 hours, after LKN.

49. The method of claim 1, wherein the VLA-4 antagonist is an anti-VLA-4 antibody molecule, e.g., an anti-VLA-4 antibody molecule described herein.

50. The method of claim 49, wherein the anti-VLA-4 antibody molecule is a monoclonal, a humanized, a human, a chimeric anti-VLA-4 antibody molecule.

51. The method of claim 49, wherein the VLA-4 antagonist is an α4-binding fragment of an anti-VLA-4 antibody.

52. The method of claim 51, wherein the α4 binding fragment is an Fab, Fab′, F(ab′)₂, or Fv fragment.

53. The method of claim 1, wherein the anti-VLA-4 antibody molecule comprises one or more, preferably all, of HC CDR1, HC CDR2, HC CDR3, LC CDR1, LC CDR2 and LC CDR3 of natalizumab.

54. The method of claim 1, wherein the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

55. The method of claim 1, wherein the stroke is a embolism-, thrombus- or hypoperfusion-associated stroke.

56. The method of claim 1, wherein the subject having the stroke does not have an intracranial hemorrhage.

57. The method of claim 1, wherein the subject has not received a previous treatment with a VLA-4 antagonist, e.g., natalizumab.

58. The method of claim 1, wherein the subject does not have or is not at risk for developing progressive multifocal leukoencephalopathy (PML).

59. The method of claim 1, wherein the VLA-4 antagonist is administered in combination with an additional agent or procedure.

60. The method of claim 59, wherein the VLA-4 antagonist is administered simultaneously with an additional agent or procedure.

61. The method of claim 59, wherein the VLA-4 antagonist is administered sequentially with an additional agent or procedure.

62. The method of claim 61, wherein the VLA-4 antagonist is administered, e.g., 30 minutes, 1 hour, 2 hours, 3 hours, 4 hours, 5 hours, or more, after the additional agent or procedure.

63. The method of claim 61, wherein the VLA-4 antagonist is administered, e.g., 30 minutes, 1 hour, 2 hours, 3 hours, 4 hours, 5 hours, or more, before the additional agent or procedure.

64. The method of claim 59, wherein the additional agent ameliorates one or more side effected associated with the administration of the VLA-4 antagonist, e.g., an agent which reduces or inhibits one or more symptom of hypersensitivity.

65. The method of claim 64, wherein the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine).

66. The method of claim 59, wherein the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

67. The method of claim 1, wherein the VLA-4 antagonist is administered at a dosage and/or dosing schedule described herein.

68. The method of claim 1, wherein the VLA-4 antagonist is administered intravenously.

69. A method of treating a human subject having a severe stroke, e.g., a severe ischemic stroke, e.g., a severe acute ischemic stroke, or a severe hemorrhagic stroke, e.g., a severe acute intracerebral hemorrhage, or a severe subarachnoid hemorrhage, comprising:
administering a VLA-4 antagonist, e.g., an anti-alpha4 antibody molecule, e.g., a natalizumab-like antibody molecule, e.g., natalizumab, to the subject,
thereby treating the human subject.

70. The method of claim 69, comprising:
determining, e.g., determining prior to administration of the VLA-4 antagonist, if the patient has a severe stroke.

71. The method of claim 70, wherein determining comprises determining a NIHSS score, e.g., determining if the NIHSS score is equal to or greater than 15 or 21.

72. The method of claim 70, wherein determining comprises determining if the NIHSS score is equal to or greater than 15.

73. The method of claim 70, wherein determining comprises determining if the NIHSS score is equal to or greater than 21.

74. The method of claim 69, wherein the subject, e.g., at baseline, e.g., after stroke onset but prior to treatment with a natalizumab-like antibody molecule, e.g., natalizumab, has a NIHSS score of equal to or greater than 15 or 21, e.g., 21 to 26.

75. The method of claim 74, wherein the NIHSS score is equal to or greater than 15.

76. The method of claim 74, wherein the NIHSS score is equal to or greater than 21.

77. The method of claim 69, wherein administration is initiated within 6 hours of last known normal.

78. The method of claim 69, wherein administration is initiated within 9 hours, e.g., 6 to 9 hours, of last known normal.

79. The method of claim 69, wherein administration is initiated within 12 hours, e.g., 6 to 12 hours, or 9 to 12 hours, of last known normal.

80. The method of claim 69, further comprising providing a subsequent administration of the VLA-4 antagonist to the subject.

81. The method of claim 69, wherein the VLA-4 antagonist comprises and antibody molecule that comprises CDR1, CDR2 and CDR3 from the light chain and CDR1, CDR2 and CDR3 from the heavy chain of natalizumab.

82. The method of claim 69, wherein the VLA-4 antagonist comprises and antibody molecule that comprises the light chain variable region and the heavy chain variable region of natalizumab.

83. The method of claim 69, wherein the VLA-4 antagonist comprises natalizumab.

84. The method of claim 69, comprising administering a VLA-4 antagonist to the subject within 12 hours or less, e.g., 10, 9, 8, 7, 6 hours or less, after LKN in the subject.

85. The method of claim 69, wherein the VLA-4 antagonist is administered within 9 hours or less after LKN, e.g., within 9, 8, 7, or 6 hours or less after LKN, or between 6 and 9 hours after LKN, or within 6 hours after LKN.

86. The method of claim 69, wherein the VLA-4 antagonist is administered within 6 hours or less after LKN, e.g., between 3 and 6 hours, 4.5 to 6 hours, 5 to 6 hours, after LKN.

87. The method of claim 69, comprising administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject.

88. The method of claim 69, wherein the VLA-4 antagonist is administered within more than 2 to 9 hours or less after LKN, e.g., between 6 and 9 hours after LKN.

89. The method of claim 69, wherein the VLA-4 antagonist is administered within more than 2 hours to 6 hours or less after LKN, e.g., between 3 and 6 hours, 4.5 to 6 hours, 5 to 6 hours, after LKN.

90. The method of claim 69, wherein the VLA-4 antagonist is an anti-VLA-4 antibody molecule, e.g., an anti-VLA-4 antibody molecule described herein.

91. The method of claim 90, wherein the anti-VLA-4 antibody molecule is a monoclonal, a humanized, a human, a chimeric anti-VLA-4 antibody molecule.

92. The method of claim 90, wherein the VLA-4 antagonist is an α4-binding fragment of an anti-VLA-4 antibody.

93. The method of claim 92, wherein the α4 binding fragment is an Fab, Fab′, F(ab′)₂, or Fv fragment.

94. The method of claim 69, wherein the anti-VLA-4 antibody molecule comprises one or more, preferably all, of HC CDR1, HC CDR2, HC CDR3, LC CDR1, LC CDR2 and LC CDR3 of natalizumab.

95. The method of claim 69, wherein the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

96. The method of claim 69, wherein the stroke is a embolism-, thrombus- or hypoperfusion-associated stroke.

97. The method of claim 69, wherein the subject having the stroke does not have an intracranial hemorrhage.

98. The method of claim 69, wherein the subject has not received a previous treatment with a VLA-4 antagonist, e.g., natalizumab.

99. The method of claim 69, wherein the subject does not have or is not at risk for developing progressive multifocal leukoencephalopathy (PML).

100. The method of claim 69, wherein the VLA-4 antagonist is administered in combination with an additional agent or procedure.

101. The method of claim 100, wherein the VLA-4 antagonist is administered simultaneously with an additional agent or procedure.

102. The method of claim 100, wherein the VLA-4 antagonist is administered sequentially with an additional agent or procedure.

103. The method of claim 102, wherein the VLA-4 antagonist is administered, e.g., 30 minutes, 1 hour, 2 hours, 3 hours, 4 hours, 5 hours, or more, after the additional agent or procedure.

104. The method of claim 102, wherein the VLA-4 antagonist is administered, e.g., 30 minutes, 1 hour, 2 hours, 3 hours, 4 hours, 5 hours, or more, before the additional agent or procedure.

105. The method of claim 100, wherein the additional agent ameliorates one or more side effected associated with the administration of the VLA-4 antagonist, e.g., an agent which reduces or inhibits one or more symptom of hypersensitivity.

106. The method of claim 105, wherein the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine).

107. The method of claim 100, wherein the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

108. The method of claim 69, wherein the VLA-4 antagonist is administered at a dosage and/or dosing schedule described herein.

109. The method of claim 69, wherein the VLA-4 antagonist is administered intravenously.

110. A method of treating a human subject having a stroke, e.g., an ischemic stroke, e.g., an acute ischemic stroke, or a hemorrhagic stroke, e.g., an intracerebral hemorrhage, or a subarachnoid hemorrhage, comprising:
administering a VLA-4 antagonist, e.g., an anti-alpha4 antibody molecule, e.g., a natalizumab like antibody molecule, e.g., natalizumab, to the subject, at a dosage of
i) 250 to 390, e.g., 275 to 325, e.g., 300+/−5%, or about 300, e.g., 300, mg;
ii) a dosage that results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L; or
iii) a dosage that results in an AUC that is comparable or higher than (e.g., within 5-10% of) the median exposure observed in lower body weight (e.g., body weight of <80 kg) or less severe stroke patients;
thereby treating the human subject.

111. The method of claim 110, comprising:
determining, e.g., determining prior to administration of the VLA-4 antagonist, if the patient has a less than severe stroke.

112. The method of claim 111, wherein determining comprises determining NIHSS score, e.g., determining if the NIHSS score is less than 15.

113. The method of claim 110, wherein the subject has a less than severe stroke.

114. The method of claim 110, wherein the subject, e.g., at baseline, e.g., after stroke onset but prior to treatment with a natalizumab-like antibody molecule, e.g., natalizumab, has a NIHSS score of 3 to 7.

115. The method of claim 110, wherein the subject, e.g., at baseline, e.g., after stroke onset but prior to treatment with a natalizumab-like antibody molecule, e.g., natalizumab, has a NIHSS score of 8 to 10.

116. The method of claim 110, wherein the subject, e.g., at baseline, e.g., after stroke onset but prior to treatment with a natalizumab-like antibody molecule, e.g., natalizumab, has a NIHSS score of 11 to 14.

117. The method of claim 110, wherein the subject, e.g., at baseline, e.g., after stroke onset but prior to treatment with a natalizumab-like antibody molecule, e.g., natalizumab, has a NIHSS score of 15 to 14.

118. The method of claim 110, wherein administration is initiated within 6 hours of last known normal.

119. The method of claim 110, wherein administration is initiated within 9 hours, e.g., 6 to 9 hours, of last known normal.

120. The method of claim 110, wherein administration is initiated within 12 hours, e.g., 6 to 12 hours, or 9 to 12 hours, of last known normal.

121. The method of claim 110, wherein the dosage is about 300 mg.

122. The method of claim 110, wherein the VLA-4 antagonist comprises and antibody molecule that comprises CDR1, CDR2 and CDR3 from the light chain and CDR1, CDR2 and CDR3 from the heavy chain of natalizumab.

123. The method of claim 110, wherein the VLA-4 antagonist comprises and antibody molecule that comprises the light chain variable region and the heavy chain variable region of natalizumab.

124. The method of claim 110, wherein the VLA-4 antagonist comprises natalizumab.

125. The method of claim 110, comprising administering a VLA-4 antagonist to the subject within 12 hours or less, e.g., 10, 9, 8, 7, 6 hours or less, after LKN in the subject.

126. The method of claim 110, wherein the VLA-4 antagonist is administered within 9 hours or less after LKN, e.g., within 9, 8, 7, or 6 hours or less after LKN, or between 6 and 9 hours after LKN, or within 6 hours after LKN.

127. The method of claim 110, wherein the VLA-4 antagonist is administered within 6 hours or less after LKN, e.g., between 3 and 6 hours, 4.5 to 6 hours, 5 to 6 hours, after LKN.

128. The method of claim 110, comprising administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject.

129. The method of claim 110, wherein the VLA-4 antagonist is administered within more than 2 to 9 hours or less after LKN, e.g., between 6 and 9 hours after LKN.

130. The method of claim 110, wherein the VLA-4 antagonist is administered within more than 2 hours to 6 hours or less after LKN, e.g., between 3 and 6 hours, 4.5 to 6 hours, 5 to 6 hours, after LKN.

131. The method of claim 110, wherein the VLA-4 antagonist is an anti-VLA-4 antibody molecule, e.g., an anti-VLA-4 antibody molecule described herein.

132. The method of claim 131, wherein the anti-VLA-4 antibody molecule is a monoclonal, a humanized, a human, a chimeric anti-VLA-4 antibody molecule.

133. The method of claim 131, wherein the VLA-4 antagonist is an α4-binding fragment of an anti-VLA-4 antibody.

134. The method of claim 133, wherein the α4 binding fragment is an Fab, Fab′, F(ab′)₂, or Fv fragment.

135. The method of claim 110, wherein the anti-VLA-4 antibody molecule comprises one or more, preferably all, of HC CDR1, HC CDR2, HC CDR3, LC CDR1, LC CDR2 and LC CDR3 of natalizumab.

136. The method of claim 110, wherein the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

137. The method of claim 110, wherein the stroke is a embolism-, thrombus- or hypoperfusion-associated stroke.

138. The method of claim 110, wherein the subject having the stroke does not have an intracranial hemorrhage.

139. The method of claim 110, wherein the subject has not received a previous treatment with a VLA-4 antagonist, e.g., natalizumab.

140. The method of claim 110, wherein the subject does not have or is not at risk for developing progressive multifocal leukoencephalopathy (PML).

141. The method of claim 110, wherein the VLA-4 antagonist is administered in combination with an additional agent or procedure.

142. The method of claim 141, wherein the VLA-4 antagonist is administered simultaneously with an additional agent or procedure.

143. The method of claim 141, wherein the VLA-4 antagonist is administered sequentially with an additional agent or procedure.

144. The method of claim 143, wherein the VLA-4 antagonist is administered, e.g., 30 minutes, 1 hour, 2 hours, 3 hours, 4 hours, 5 hours, or more, after the additional agent or procedure.

145. The method of claim 143, wherein the VLA-4 antagonist is administered, e.g., 30 minutes, 1 hour, 2 hours, 3 hours, 4 hours, 5 hours, or more, before the additional agent or procedure.

146. The method of claim 141, wherein the additional agent ameliorates one or more side effected associated with the administration of the VLA-4 antagonist, e.g., an agent which reduces or inhibits one or more symptom of hypersensitivity.

147. The method of claim 146, wherein the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine).

148. The method of claim 141, wherein the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

149. The method of claim 110, wherein the VLA-4 antagonist is administered at a dosage and/or dosing schedule described herein.

150. The method of claim 110, wherein the VLA-4 antagonist is administered intravenously.

151. A method of treating a human subject having a less than severe stroke, e.g., a less than severe ischemic stroke, e.g., a less than severe acute ischemic stroke, or a less than severe hemorrhagic stroke, e.g., a less than severe intracerebral hemorrhage, or a subarachnoid hemorrhage, comprising:
administering a VLA-4 antagonist, e.g., an anti-alpha4 antibody molecule, e.g., a natalizumab-like antibody molecule, e.g., natalizumab, to the subject.
thereby treating the human subject.

152. The method of claim 151, comprising:
determining, e.g., determining prior to administration of the VLA-4 antagonist, if the patient has a less than severe stroke.

153. The method of claim 152, wherein determining comprises determining NIHSS score, e.g., determining if the NIHSS score is less than 15.

154. The method of claim 151, wherein the subject, e.g., at baseline, e.g., after stroke onset but prior to treatment with the VLA-4 antagonist has a NIHSS score of 3 to 7.

155. The method of claim 151, wherein the subject, e.g., at baseline, e.g., after stroke onset but prior to treatment with the VLA-4 antagonist has a NIHSS score of 8 to 10.

156. The method of claim 151, wherein the subject, e.g., at baseline, e.g., after stroke onset but prior to treatment with the VLA-4 antagonist has a NIHSS score of 11 to 14.

157. The method of claim 151, wherein administration is initiated within 6 hours of last known normal.

158. The method of claim 151, wherein administration is initiated within 9 hours, e.g., 6 to 9 hours, of last known normal.

159. The method of claim 151, wherein administration is initiated within 12 hours, e.g., 6 to 12 hours, or 9 to 12 hours, of last known normal.

160. The method of claim 151, further comprising providing a subsequent administration of the VLA-4 antagonist to the subject.

161. The method of claim 151, wherein the VLA-4 antagonist comprises and antibody molecule that comprises CDR1, CDR2 and CDR3 from the light chain and CDR1, CDR2 and CDR3 from the heavy chain of natalizumab.

162. The method of claim 151, wherein the VLA-4 antagonist comprises and antibody molecule that comprises the light chain variable region and the heavy chain variable region of natalizumab.

163. The method of claim 151, wherein the VLA-4 antagonist comprises natalizumab.

164. The method of claim 151, comprising administering a VLA-4 antagonist to the subject within 12 hours or less, e.g., 10, 9, 8, 7, 6 hours or less, after LKN in the subject.

165. The method of claim 151, wherein the VLA-4 antagonist is administered within 9 hours or less after LKN, e.g., within 9, 8, 7, or 6 hours or less after LKN, or between 6 and 9 hours after LKN, or within 6 hours after LKN.

166. The method of claim 151, wherein the VLA-4 antagonist is administered within 6 hours or less after LKN, e.g., between 3 and 6 hours, 4.5 to 6 hours, 5 to 6 hours, after LKN.

167. The method of claim 151, comprising administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject.

168. The method of claim 151, wherein the VLA-4 antagonist is administered within more than 2 to 9 hours or less after LKN, e.g., between 6 and 9 hours after LKN.

169. The method of claim 151, wherein the VLA-4 antagonist is administered within more than 2 hours to 6 hours or less after LKN, e.g., between 3 and 6 hours, 4.5 to 6 hours, 5 to 6 hours, after LKN.

170. The method of claim 151, wherein the VLA-4 antagonist is an anti-VLA-4 antibody molecule, e.g., an anti-VLA-4 antibody molecule described herein.

171. The method of claim 170, wherein the anti-VLA-4 antibody molecule is a monoclonal, a humanized, a human, a chimeric anti-VLA-4 antibody molecule.

172. The method of claim 170, wherein the VLA-4 antagonist is an α4-binding fragment of an anti-VLA-4 antibody.

173. The method of claim 172, wherein the α4 binding fragment is an Fab, Fab′, F(ab′)₂, or Fv fragment.

174. The method of claim 151, wherein the anti-VLA-4 antibody molecule comprises one or more, preferably all, of HC CDR1, HC CDR2, HC CDR3, LC CDR1, LC CDR2 and LC CDR3 of natalizumab.

175. The method of claim 151, wherein the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

176. The method of claim 151, wherein the stroke is a embolism-, thrombus- or hypoperfusion-associated stroke.

177. The method of claim 151, wherein the subject having the stroke does not have an intracranial hemorrhage.

178. The method of claim 151, wherein the subject has not received a previous treatment with a VLA-4 antagonist, e.g., natalizumab.

179. The method of claim 151, wherein the subject does not have or is not at risk for developing progressive multifocal leukoencephalopathy (PML).

180. The method of claim 151, wherein the VLA-4 antagonist is administered in combination with an additional agent or procedure.

181. The method of claim 180, wherein the VLA-4 antagonist is administered simultaneously with an additional agent or procedure.

182. The method of claim 180, wherein the VLA-4 antagonist is administered sequentially with an additional agent or procedure.

183. The method of claim 182, wherein the VLA-4 antagonist is administered, e.g., 30 minutes, 1 hour, 2 hours, 3 hours, 4 hours, 5 hours, or more, after the additional agent or procedure.

184. The method of claim 182, wherein the VLA-4 antagonist is administered, e.g., 30 minutes, 1 hour, 2 hours, 3 hours, 4 hours, 5 hours, or more, before the additional agent or procedure.

185. The method of claim 180, wherein the additional agent ameliorates one or more side effected associated with the administration of the VLA-4 antagonist, e.g., an agent which reduces or inhibits one or more symptom of hypersensitivity.

186. The method of claim 185, wherein the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine).

187. The method of claim 180, wherein the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

188. The method of claim 151, wherein the VLA-4 antagonist is administered at a dosage and/or dosing schedule described herein.

189. The method of claim 151, wherein the VLA-4 antagonist is administered intravenously.

190. A method of treating a human subject having a stroke, e.g., an ischemic stroke, e.g., an acute ischemic stroke, or hemorrhagic stroke, e.g., an intracerebral hemorrhage, or a subarachnoid hemorrhage, comprising:
determining the severity of a stroke, e.g., by receiving information, e.g., from a third party, on the severity of the stroke, and responsive to that determination, selecting a dosage of a VLA-4 antagonist, e.g., an anti-alpha4 antibody molecule, e.g., a natalizumab-like antibody molecule, e.g., natalizumab,
thereby treating the human subject.

191. The method of claim 190, further comprising administering the selected dosage to the subject.

192. The method of claim 190, wherein if the stroke is determined to be a severe stroke, selecting and/or administering a dosage-A of the VLA-4 antagonist and if the stroke is determined to be a less than severe stroke, selecting and or administering a dosage-B, e.g., wherein the dosage-A is higher than dosage-B, e.g., is at least 10, 20 or 30% higher.

193. The method of claim 192, wherein dosage-A is 350 to 500, e.g., 390 to 450, or 450+/−5%, or about 450, e.g., 450, mg; 550 to 650, e.g., 575 to 625, e.g., 600+/−5%, e.g., about 600, e.g., 600, mg.

194. The method of claim 193, wherein dosage-A is 350 to 500 mg.

195. The method of claim 193, wherein dosage-A is 390 to 450 mg.

196. The method of claim 193, wherein dosage-A is 450+/−5% mg.

197. The method of claim 193, wherein dosage-A is about 450 mg.

198. The method of claim 193, wherein dosage-A is 450 mg.

199. The method of claim 193, wherein dosage-A is 550 to 650 mg.

200. The method of claim 193, wherein dosage-A is 575 to 625 mg.

201. The method of claim 193, wherein dosage-A is 600+/−5% mg.

202. The method of claim 193, wherein dosage-A is about 600 mg.

203. The method of claim 193, wherein dosage-A is 600 mg.

204. The method of claim 192, wherein dosage-A results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L; or
wherein dosage-A is results in an AUC that is comparable or higher than (e.g., within 5-10% of) the median exposure observed in lower body weight (e.g., body weight of <80 kg) or less severe stroke patients.

205. The method of claim 192, wherein dosage-B is 250 to 390, e.g., 275 to 325, e.g., 300+/−5%, or about 300, e.g., 300, mgs.

206. The method of claim 205, wherein dosage-B is 250 to 390 mg.

207. The method of claim 205, wherein dosage-B is 275 to 325 mg.

208. The method of claim 205, wherein dosage-B is 300+/−5% mg.

209. The method of claim 205, wherein dosage-B is about 300 mg.

210. The method of claim 205, wherein dosage-B is 300 mg.

211. The method of claim 192, wherein dosage-B results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000, mg*hr/L; or
wherein dosage-B is results in an AUC thatis comparable or higher than (e.g., within 5-10% of) the median exposure observed in lower body weight (e.g., body weight of <80 kg) or less severe stroke patients.

212. The method of claim 190, wherein determining comprises determining a NIHSS score, e.g., determining if the NIHSS score is equal to or greater than 15 or 21.

213. The method of claim 212, wherein determining comprises determining if the NIHSS score is equal to or greater than 15.

214. The method of claim 212, wherein determining comprises determining if the NIHSS score is equal to or greater than 21.

215. The method of claim 190, comprising determining that the stroke is a severe stroke.

216. The method of claim 190, wherein the subject, e.g., at baseline, e.g., after stroke onset but prior to treatment with a natalizumab-like antibody molecule, e.g., natalizumab, has a NIHSS score of 21 or greater, e.g., 21 to 26.

217. The method of claim 216, wherein the NIHSS score is equal to or greater than 15.

218. The method of claim 216, wherein the NIHSS score is equal to or greater than 21.

219. The method of claim 190, wherein determining comprises determining NIHSS score, e.g., determining if the NIHSS score is less than 15.

220. The method of claim 190, wherein the stroke is determined to be less than severe.

221. The method of claim 190, wherein the subject, e.g., at baseline, e.g., after stroke onset but prior to treatment with a natalizumab-like antibody molecule, e.g., natalizumab, has a NIHSS score of 3 to 7.

222. The method of claim 190, wherein the subject, e.g., at baseline, e.g., after stroke onset but prior to treatment with a natalizumab-like antibody molecule, e.g., natalizumab, has a NIHSS score of 8 to 10.

223. The method of claim 190, wherein the subject, e.g., at baseline, e.g., after stroke onset but prior to treatment with a natalizumab-like antibody molecule, e.g., natalizumab, has a NIHSS score of 11 to 14.

224. The method of claim 190, wherein the subject, e.g., at baseline, e.g., after stroke onset but prior to treatment with a natalizumab-like antibody molecule, e.g., natalizumab, has a NIHSS score of 5 to 14.

225. The method of claim 190, wherein administration is initiated within 6 hours of last known normal.

226. The method of claim 190, wherein administration is initiated within 9 hours, e.g., 6 to 9 hours, of last known normal.

227. The method of claim 190, wherein administration is initiated within 12 hours, e.g., 6 to 12 hours, or 9 to 12 hours, of last known normal.

228. The method of claim 190, wherein the VLA-4 antagonist comprises and antibody molecule that comprises CDR1, CDR2 and CDR3 from the light chain and CDR1, CDR2 and CDR3 from the heavy chain of natalizumab.

229. The method of claim 190, wherein the VLA-4 antagonist comprises and antibody molecule that comprises the light chain variable region and the heavy chain variable region of natalizumab.

230. The method of claim 190, wherein the VLA-4 antagonist comprises natalizumab.

231. The method of claim 190, comprising administering a VLA-4 antagonist to the subject within 12 hours or less, e.g., 10, 9, 8, 7, 6 hours or less, after LKN in the subject.

232. The method of claim 190, wherein the VLA-4 antagonist is administered within 9 hours or less after LKN, e.g., within 9, 8, 7, or 6 hours or less after LKN, or between 6 and 9 hours after LKN, or within 6 hours after LKN.

233. The method of claim 190, wherein the VLA-4 antagonist is administered within 6 hours or less after LKN, e.g., between 3 and 6 hours, 4.5 to 6 hours, 5 to 6 hours, after LKN.

234. The method of claim 190, comprising administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject.

235. The method of claim 190, wherein the VLA-4 antagonist is administered within more than 2 to 9 hours or less after LKN, e.g., between 6 and 9 hours after LKN.

236. The method of claim 190, wherein the VLA-4 antagonist is administered within more than 2 hours to 6 hours or less after LKN, e.g., between 3 and 6 hours, 4.5 to 6 hours, 5 to 6 hours, after LKN.

237. The method of claim 190, wherein the VLA-4 antagonist is an anti-VLA-4 antibody molecule, e.g., an anti-VLA-4 antibody molecule described herein.

238. The method of claim 237, wherein the anti-VLA-4 antibody molecule is a monoclonal, a humanized, a human, a chimeric anti-VLA-4 antibody molecule.

239. The method of claim 237, wherein the VLA-4 antagonist is an α4-binding fragment of an anti-VLA-4 antibody.

240. The method of claim 239, wherein the α4 binding fragment is an Fab, Fab′, F(ab′)₂, or Fv fragment.

241. The method of claim 190, wherein the anti-VLA-4 antibody molecule comprises one or more, preferably all, of HC CDR1, HC CDR2, HC CDR3, LC CDR1, LC CDR2 and LC CDR3 of natalizumab.

242. The method of claim 190, wherein the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

243. The method of claim 190, wherein the stroke is a embolism-, thrombus- or hypoperfusion-associated stroke.

244. The method of claim 190, wherein the subject having the stroke does not have an intracranial hemorrhage.

245. The method of claim 190, wherein the subject has not received a previous treatment with a VLA-4 antagonist, e.g., natalizumab.

246. The method of claim 190, wherein the subject does not have or is not at risk for developing progressive multifocal leukoencephalopathy (PML).

247. The method of claim 190, wherein the VLA-4 antagonist is administered in combination with an additional agent or procedure.

248. The method of claim 247, wherein the VLA-4 antagonist is administered simultaneously with an additional agent or procedure.

249. The method of claim 247, wherein the VLA-4 antagonist is administered sequentially with an additional agent or procedure.

250. The method of claim 249, wherein the VLA-4 antagonist is administered, e.g., 30 minutes, 1 hour, 2 hours, 3 hours, 4 hours, 5 hours, or more, after the additional agent or procedure.

251. The method of claim 249, wherein the VLA-4 antagonist is administered, e.g., 30 minutes, 1 hour, 2 hours, 3 hours, 4 hours, 5 hours, or more, before the additional agent or procedure.

252. The method of claim 247, wherein the additional agent ameliorates one or more side effected associated with the administration of the VLA-4 antagonist, e.g., an agent which reduces or inhibits one or more symptom of hypersensitivity.

253. The method of claim 252, wherein the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine).

254. The method of claim 247, wherein the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

255. The method of claim 190, wherein the VLA-4 antagonist is administered at a dosage and/or dosing schedule described herein.

256. The method of claim 190, wherein the VLA-4 antagonist is administered intravenously.

257. A method of treating a human subject having a stroke, e.g., an ischemic stroke, e.g., an acute ischemic stroke, or a hemorrhagic stroke, e.g., an intracerebral hemorrhage, or a subarachnoid hemorrhage, comprising:
administering a first dosage of a VLA-4 antagonist to the subject at a time t1,
administering a second dosage of a VLA-4 antagonist to the subject at a time t2, and (optionally)
administering a third dosage of a VLA-4 antagonist to the subject at a time t3, thereby treating the subject.

258. The method of claim 257, comprising administering a third dosage of a VLA-4 antagonist to the subject at a time t3.

259. The method of claim 257, wherein the three dosages result in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L.

260. The method of claim 257, wherein the first dosage is greater than one or both of the second and third dosages.

261. The method of claim 257, wherein the first dosage is the same as one or both of the second and third dosages, e.g., wherein all three dosages are the same.

262. The method of claim 257, wherein t1 is the day (i.e., within 24 hours) of diagnosis.

263. The method of claim 257, wherein at least one or more days (e.g., 1, 2, 3, 4, 5, or more days) are interposed between t1 and t2.

264. The method of claim 263, wherein about 2 days (e.g., 36-60 hours, e.g., 40-56 hours, e.g., 44-52 hours, e.g., about 48 hours) are interposed between t1 and t2.

265. The method of claim 257, wherein at least one or more days (e.g., 1, 2, 3, 4, 5, or more days) are interposed between t2 and t3.

266. The method of claim 257, wherein about 2 days (e.g., 36-60 hours, e.g., 40-56 hours, e.g., 44-52 hours, e.g., about 48 hours) are interposed between t2 and t3.

267. The method of claim 257, wherein:
t1 is the day on which the first dosage is administered;
g2 is the third day after t1, (e.g., if t1 is a Monday, then t2 is the first following Wednesday); and t3 is the fifth day after t1.

268. The method of claim 257, wherein the first dosage is 250 to 350 mg, the second dosage is 100 to 200 mg, and the third dosage is 100 to 200 mg.

269. The method of claim 257, wherein the first dosage is 275 to 325 mg, the second dosage is 125 mg to 175 mg, and the third dosage is 125 to 175 mg.

270. The method of claim 257, wherein the first dosage is 300+/−5% mg, the second dosage is 150+/−5% mg, and the third dosage is 150+/−5% mg.

271. The method of claim 257, wherein the first dosage is about 300 mg, the second dosage is about 150 mg, and the third dosage is about 150 mg.

272. The method of claim 257, wherein the first dosage is 300 mg, the second dosage is 150 mg, and the third dosage is 150 mg.

273. The method of claim 257, wherein the first, second and third dosage, each is 100 to 200 mg.

274. The method of claim 257, wherein the first, second and third dosage, each is 125 to 175 mg.

275. The method of claim 257, wherein the first, second and third dosage, each is 150+/−5% mg.

276. The method of claim 257, wherein the first, second and third dosage, each is about 150 mg.

277. The method of claim 257, wherein the first, second and third dosage, each is 150 mg.

278. The method of claim 257, wherein the subject has a severe stroke, e.g., a stroke having a NIHSS score equal to or greater than 15 or 21.

279. The method of claim 278, wherein the NIHSS score is equal to or greater than 15.

280. The method of claim 278, wherein the NIHSS score is equal to or greater than 21.

281. The method of claim 257, wherein the subject has a less than severe stroke, e.g., a stroke having a NIHSS score of less than 15.

282. The method of claim 257, comprising:
determining, e.g., determining prior to administration of the VLA-4 antagonist, the severity of the stroke, e.g., determining if the patient has a severe stroke.

283. The method of claim 282, wherein determining comprises determining a NIHSS score, e.g., determining if the NIHSS score is equal to or greater than 15 or 21.

284. The method of claim 283, wherein determining comprises determining if the NIHSS score is equal to or greater than 15.

285. The method of claim 283, wherein determining comprises determining if the NIHSS score is equal to or greater than 21.

286. The method of claim 257, comprising:
determining, e.g., determining prior to administration of the VLA-4 antagonist, the severity of the stroke, e.g., determining if the patient has less than severe stroke.

287. The method of claim 286, wherein determining comprises determining a NIHSS score, e.g., determining if the NIHSS score is less than 15.

288. The method of claim 282, wherein responsive to the determination of severity, a dosage of anti-VLA4 antagonist is selected.

289. The method of claim 257, wherein administration of the first dosage is initiated within 6 hours of last known normal.

290. The method of claim 257, wherein administration of the first dosage is initiated within 9 hours, e.g., 6 to 9 hours, of last known normal.

291. The method of claim 257, wherein administration of the first dosage is initiated within 12 hours, e.g., 6 to 12 hours, or 9 to 12 hours, of last known normal.

292. The method of claim 257, wherein the VLA-4 antagonist comprises natalizumab.

293. The method of claim 257, comprising administering a VLA-4 antagonist to the subject within 12 hours or less, e.g., 10, 9, 8, 7, 6 hours or less, after LKN in the subject.

294. The method of claim 257, wherein the VLA-4 antagonist is administered within 9 hours or less after LKN, e.g., within 9, 8, 7, or 6 hours or less after LKN, or between 6 and 9 hours after LKN, or within 6 hours after LKN.

295. The method of claim 257, wherein the VLA-4 antagonist is administered within 6 hours or less after LKN, e.g., between 3 and 6 hours, 4.5 to 6 hours, 5 to 6 hours, after LKN.

296. The method of claim 257, comprising administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after LKN in the subject.

297. The method of claim 257, wherein the VLA-4 antagonist is administered within more than 2 to 9 hours or less after LKN, e.g., between 6 and 9 hours after LKN.

298. The method of claim 257, wherein the VLA-4 antagonist is administered within more than 2 hours to 6 hours or less after LKN, e.g., between 3 and 6 hours, 4.5 to 6 hours, 5 to 6 hours, after LKN.

299. The method of claim 257, wherein the VLA-4 antagonist is an anti-VLA-4 antibody molecule, e.g., an anti-VLA-4 antibody molecule described herein.

300. The method of claim 299, wherein the anti-VLA-4 antibody molecule is a monoclonal, a humanized, a human, a chimeric anti-VLA-4 antibody molecule.

301. The method of claim 299, wherein the VLA-4 antagonist is an α4-binding fragment of an anti-VLA-4 antibody.

302. The method of claim 301, wherein the α4 binding fragment is an Fab, Fab′, F(ab′)₂, or Fv fragment.

303. The method of claim 257, wherein the anti-VLA-4 antibody molecule comprises one or more, preferably all, of HC CDR1, HC CDR2, HC CDR3, LC CDR1, LC CDR2 and LC CDR3 of natalizumab.

304. The method of claim 257, wherein the VLA-4 antagonist, e.g., an anti-alpha4 antibody molecule, e.g., a natalizumab-like antibody molecule, e.g., natalizumab, is administered by intravenous administration, e.g., over a period of less than 90 minutes, e.g., 30 to 60 minutes.

305. The method of claim 257, wherein the stroke is a embolism-, thrombus- or hypoperfusion-associated stroke.

306. The method of claim 257, wherein the subject having the stroke does not have an intracranial hemorrhage.

307. The method of claim 257, wherein the subject has not received a previous treatment with a VLA-4 antagonist, e.g., natalizumab.

308. The method of claim 257, wherein the subject does not have or is not at risk for developing progressive multifocal leukoencephalopathy (PML).

309. The method of claim 257, wherein the VLA-4 antagonist is administered in combination with an additional agent or procedure.

310. The method of claim 309, wherein the VLA-4 antagonist is administered simultaneously with an additional agent or procedure.

311. The method of claim 309, wherein the VLA-4 antagonist is administered sequentially with an additional agent or procedure.

312. The method of claim 311, wherein the VLA-4 antagonist is administered, e.g., 30 minutes, 1 hour, 2 hours, 3 hours, 4 hours, 5 hours, or more, after the additional agent or procedure.

313. The method of claim 311, wherein the VLA-4 antagonist is administered, e.g., 30 minutes, 1 hour, 2 hours, 3 hours, 4 hours, 5 hours, or more, before the additional agent or procedure.

314. The method of claim 309, wherein the additional agent ameliorates one or more side effected associated with the administration of the VLA-4 antagonist, e.g., an agent which reduces or inhibits one or more symptom of hypersensitivity.

315. The method of claim 314, wherein the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine).

316. The method of claim 309, wherein the additional agent is an agent which reduces one of more symptom of stroke, e.g., ischemic stroke (e.g., acute ischemic stroke), or hemorrhagic stroke (e.g., intracerebral hemorrhage), or subarachnoid hemorrhage, or TBI.

317. The method of claim 257, wherein the VLA-4 antagonist is administered at a dosage and/or dosing schedule described herein.

318. The method of claim 257, wherein the VLA-4 antagonist is administered intravenously.

319. The method of claim 257, wherein the VLA-4 antagonist comprises and antibody molecule that comprises CDR1, CDR2 and CDR3 from the light chain and CDR1, CDR2 and CDR3 from the heavy chain of natalizumab.

320. The method of claim 257, wherein the VLA-4 antagonist comprises and antibody molecule that comprises the light chain variable region and the heavy chain variable region of natalizumab.

321. A method of treating a human subject having a traumatic brain injury (TBI), comprising:
administering a VLA-4 antagonist, e.g., an anti-alpha4 antibody molecule, e.g., a natalizumab-like antibody molecule, e.g., natalizumab, to the subject, at a dosage (e.g., as a single administration) of
i) 250 to 350, e.g., about 300, e.g., 300, mg; 350 to 500, e.g., 390 to 450, or 450+/−5%, or about 450, e.g., 450, mg; 550 to 650, e.g., 575 to 625, e.g., 600+/−5%, e.g., about 600, e.g., 600, mg;
ii) a dosage that results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L; or
iii) a dosage that results in an AUC that is comparable or higher than (e.g., within 5-10% of) the median exposure observed in lower body weight (e.g., body weight of <80 kg) or less severe stroke patients;
thereby treating the human subject.

322. A method of treating a human subject having a severe TBI, comprising:
administering a VLA-4 antagonist, e.g., an anti-alpha4 antibody molecule, e.g., a natalizumab-like antibody molecule, e.g., natalizumab, to the subject,
thereby treating the human subject.

323. A method of treating a human subject having a traumatic brain injury (TBI), comprising:
administering a VLA-4 antagonist, e.g., an anti-alpha4 antibody molecule, e.g., a natalizumab like antibody molecule, e.g., natalizumab, to the subject, at a dosage of
i) 250 to 390, e.g., 275 to 325, e.g., 300+/−5%, or about 300, e.g., 300, mg;
ii) a dosage that results in an AUC (e.g., over a period of time of 0-10 days, 0-20 days, 0-30 days, 0-60 days, 0-90 days, or 0-120 days) of at least about 20,000 to 30,000, e.g., at least about 20,000, 25,000, or 30,000 mg*hr/L; or
iii) a dosage that results in an AUC that is comparable or higher than (e.g., within 5-10% of) the median exposure observed in lower body weight (e.g., body weight of <80 kg) or less severe stroke patients;
thereby treating the human subject.

324. A method of treating a human subject having a less than severe TBI, comprising:
administering a VLA-4 antagonist, e.g., an anti-alpha4 antibody molecule, e.g., a natalizumab-like antibody molecule, e.g., natalizumab, to the subject.
thereby treating the human subject.

325. A method of treating a human subject having a traumatic brain injury (TBI), comprising:
determining the severity of a stroke, e.g., by receiving information, e.g., from a third party, on the severity of the stroke (or TBI), and responsive to that determination, selecting a dosage of a VLA-4 antagonist, e.g., an anti-alpha4 antibody molecule, e.g., a natalizumab-like antibody molecule, e.g., natalizumab,
thereby treating the human subject.

326. A method of treating a human subject having a traumatic brain injury (TBI), comprising:
administering a first dosage of a VLA-4 antagonist to the subject at a time t1,
administering a second dosage of a VLA-4 antagonist to the subject at a time t2, and
administering a third dosage of a VLA-4 antagonist to the subject at a time t3, thereby treating the subject.
